SEQUENCE LISTING (34 sequences)

SEQ ID NO: 1 - DNA, 687 bp, Artificial Sequence
  Synthetic Construct
taatacgactcactatagggaaatgcatgtctaagacagcatcttcaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcacctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcatgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaacaggaaattgaaaaaccagtagctgtttctgaactcttcacaactttgccaattgtacagggaaaatcaagaccaattaaaattttaactagattaacaattattgtctcggatccatctcactgcaatgttctagcataaccccttggggcctctaaacgggtcttgaggggttttttg

SEQ ID NO: 2 - DNA, 869 bp, Artificial Sequence
  Synthetic Construct
taatacgactcactatagggaaatgcatgtctaagacagcatcttcaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcacctgcaggtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcgaccttgttgatgagctggagccagagaccgacacacgggagccactgactcggatccgcaacaactcagccatccacatccgagtcttcagggtcacacccaagtaattgaaaagacactcctccacttatcccctccgtgatatggctcttcgcatgctgagtactggacctcggaccagagccatgtaagaaaaggcctgttccctggaagcccaaaggactctgcattgagggtgggggtaattgtctcttggtgggcccagttagtgggccttcctgagtgtgtgtatgcggtctgtaactattgccatataaataaaaaatcctgttgcactagtgtcctgccatccctagcataaccccttggggcctctaaacgggtcttgaggggttttttg

SEQ ID NO: 3 - protein, 11 aa, Artificial Sequence
  Synthetic Construct
TRAQRRAAARK

SEQ ID NO: 4 - DNA, 20 bp, Artificial Sequence
  Synthetic Construct
gaccccaaaatcagcgaaat

SEQ ID NO: 5 - DNA, 24 bp, Artificial Sequence
  Synthetic Construct
tctggttactgccagttgaatctg

SEQ ID NO: 6 - DNA, 24 bp, Artificial Sequence
  Synthetic Construct
accccgcattacgtttggtggacc

SEQ ID NO: 7 - DNA, 20 bp, Artificial Sequence
  Synthetic Construct
ttacaaacattggccgcaaa

SEQ ID NO: 8 - DNA, 18 bp, Artificial Sequence
  Synthetic Construct
gcgcgacattccgaagaa

SEQ ID NO: 9 - DNA, 23 bp, Artificial Sequence
  Synthetic Construct
acaatttgcccccagcgcttcag

SEQ ID NO: 10 - DNA, 18 bp, Artificial Sequence
  Synthetic Construct
gatttggacctgcgagcg

SEQ ID NO: 11 - DNA, 18 bp, Artificial Sequence
  Synthetic Construct
gcggctgtctccacaagt

SEQ ID NO: 12 - DNA, 16 bp, Artificial Sequence
  Synthetic Construct
ctgacctgaaggctct

SEQ ID NO: 13 - DNA, 28 bp, Artificial Sequence
  Synthetic Construct
gaagatcttaatacgactcactataggg

SEQ ID NO: 14 - DNA, 46 bp, Artificial Sequence
  Synthetic Construct
ttttccttttgcggccgccaaaaaacccctcaagacccgtttagag

SEQ ID NO: 15 - DNA, 49 bp, Artificial Sequence
  Synthetic Construct
cttgtgttacctgcaggtgagagcggtgaaccaagacgcagtattattg

SEQ ID NO: 16 - DNA, 49 bp, Artificial Sequence; identical to SEQ ID NO: 15
  Synthetic Construct

SEQ ID NO: 17 - DNA, 39 bp, Artificial Sequence
  Synthetic Construct
ctctcacctgcaggtaacacaagctttcggcagacgtgg

SEQ ID NO: 18 - DNA, 49 bp, Artificial Sequence
  Synthetic Construct
gctcatcaacaaggtcgacttgatctttgaaatttggatctttgtcatc

SEQ ID NO: 19 - DNA, 42 bp, Artificial Sequence
  Synthetic Construct
gatcaagtcgaccttgttgatgagctggagccagagaccgac

SEQ ID NO: 20 - DNA, 34 bp, Artificial Sequence
  Synthetic Construct
ggaattccatatgcaaaaaacccctcaagacccg

SEQ ID NO: 21 - DNA, 110 bp, Artificial Sequence
  Synthetic Construct
cgttcttcggaatgtcgcgcattggcatggaagtcacaccttcgggaacgtggttgacctacacaggtgccatcaaattggatgacaaagatccaaatttcaaagatcaa

SEQ ID NO: 22 - DNA, 110 bp, Artificial Sequence
  Synthetic Construct
ttgatctttgaaatttggatctttgtcatccaatttgatggcacctgtgtaggtcaaccacgttcccgaaggtgtgacttccatgccaatgcgcgacattccgaagaacg

SEQ ID NO: 23 - DNA, 200 bp, Artificial Sequence
  Synthetic Construct
cttgttgatgagctggagccagagaccgacacacgggagccactgactcggatccgcaacaactcagccatccacatccgagtcttcagggtcacacccaagtaattgaaaagacactcctccacttatcccctccgtgatatggctcttcgcatgctgagtactggacctcggaccagagccatgtaagaaaaggcctg

SEQ ID NO: 24 - DNA, 200 bp, Artificial Sequence
  Synthetic Construct
tcggaccagagccatgtaagaaaaggcctgttccctggaagcccaaaggactctgcattgagggtgggggtaattgtctcttggtgggcccagttagtgggccttcctgagtgtgtgtatgcggtctgtaactattgccatataaataaaaaatcctgttgcactagtgtcctgccatccctagcataaccccttggggc

SEQ ID NO: 25 - DNA, 58 bp, Artificial Sequence
  Synthetic Construct
cctgccatccctagcataaccccttggggcctctaaacgggtcttgaggggttttttg

SEQ ID NO: 26 - DNA, 28 bp, Artificial Sequence
  Synthetic Construct
gaagatcttaatacgactcactataggg

SEQ ID NO: 27 - DNA, 46 bp, Artificial Sequence; identical to SEQ ID NO: 14
  Synthetic Construct

SEQ ID NO: 28 - protein, 133 aa, Artificial Sequence
  Synthetic Construct
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 29 - DNA, 26 bp, Artificial Sequence
  Synthetic Construct
aattgcatgtctaacagacgatcttc

SEQ ID NO: 30 - protein, 190 aa, Artificial Sequence
  Synthetic Construct
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 31 - protein, 419 aa, Artificial Sequence
  Synthetic Construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 32 - DNA, 18 bp, Artificial Sequence
  Synthetic Construct
atttaggtgacactatag

SEQ ID NO: 33 - DNA, 24 bp, Artificial Sequence
  Synthetic Construct
catacgatttaggtgacactatag

SEQ ID NO: 34 - DNA, 18 bp, Artificial Sequence
  Synthetic Construct
aattaaccctcactaaag

(19) United States
(12) Patent Application Publication
(10) Pub. No.: US 2023/0272351 A1
(43) Pub. Date: Aug. 31, 2023

(54) COVID ASSAY CONTROLS

(71) Applicant: The Regents of the University of California, Oakland, CA (US)

(72) Inventors: Nicole Steinmetz, La Jolla, CA (US); Soo Khim Chan, La Jolla, CA (US)

(21) Appl. No.: 18/024,127

(22) PCT Filed: Sep. 1, 2021
(86) PCT No.: PCT/US2021/048720

§ 371 Date: Mar. 1, 2023

Related U.S. Application Data
(60) Provisional application No. 63/073,874, filed on Sep. 2, 2020.

(51) Int. Cl. C12N 7/00; C07K 14/165; C12N 15/86

(52) U.S. Cl. CPC C12N 7/00; C07K 14/165; C12N 15/86; C12N 2770/14023; C12N 2795/18023; C12N 2770/20022

(57) ABSTRACT

Provided herein is a synthetic viral particle that positive control for COVID assays, along with methods for making and using the viral particle.

### CROSS-REFERENCE TO RELATED APPLICATIONS

This application claims the benefit under 35 U.S.C. § 119(e) of U.S. Provisional Application Ser. No. 63/073,874, filed Sep. 2, 2020, the contents of which are hereby incorporated by reference into this application in its entirety.

### BACKGROUND

The ongoing coronavirus disease 2019 (COVID-19) pandemic, caused by severe acute respiratory syndrome coronavirus 2 (SARS-CoV-2), was first detected in Wuhan city, China in December 2019. As of Aug. 8, 2020, more than 19 million confirmed COVID-19 cases have been reported globally; the United States accounted for more than a quarter of the total confirmed cases and over 161,000 deaths (coronavirus.jhu.edu/map.html). SARS-CoV-2 is a betacoronavirus responsible for the COVID-19 pandemic that has led to nearly 1.5 million deaths since the outbreak began in December 2019 (COVID-19 Map—Johns Hopkins Coronavirus Resource Center available at coronavirus.jhu.edu/map.html last assessed on Nov. 15, 2020). SARS-CoV-2 demonstrates high transmissibility during the early phase of infection due to active shedding in the upper respiratory tract and droplet transmission. Accurate diagnosis of COVID-19 cannot be made clinically alone, as it exhibits clinical manifestations common to other respiratory illnesses. See, e.g., Wolfel et al., Nature 2020, 581, 465-469 and Wu et al., Emerg. Infect. Dis. 2020, 26, 1324. Moreover, asymptomatic transmission has been linked to 40-45% of COVID-19 cases. See, e.g., Oran et al., Ann. Intern. Med. 2020, M20-3012. Although promising COVID-19 vaccines are due for imminent release, control of the virus will continue to rely on the identification and containment of infected individuals, which requires accurate and widely available COVID-19 tests. See, e.g., Pokhrel et al., ACS Sensors 2020, 5, 2283-2296. Finally, COVID-19 disproportionately affects disadvantaged populations including minorities and people from lower socioeconomic classes where resources for testing are limited. See, e.g., Hooper et al., JAMA. 2020 Jun. 23; 323(24):2466-2467. Hence, the deployment of large-scale, rapid diagnostic testing is critical for widespread surveillance as well as early detection of infected individuals to mitigate the disease.

Three types of commercial COVID-19 tests are currently available: molecular diagnostic tests, antigen diagnostic tests, and antibody tests. See, e.g., Testing Data in the U.S. available at www.cdc.gov/coronavirus/2019-ncov/cases-updates/testing-in-us.html, last accessed Aug. 8, 2020. Multiple sample types are being collected from patients for testing, including nasopharyngeal swabs and saliva. See., e.g., Guglielmi, Nature 2020, 583, 506-509.

The current gold standard for the early detection of SARS-CoV-2 infections are highly specific and sensitive molecular tests based on reverse transcription polymerase chain reaction (RT-PCR). See, e.g., Udugama et al., ACS Nano 2020, 14, 3822-3835. However, RT-PCR requires sophisticated instruments, and the turnaround time is 1-2 hours. See., e.g., Carter et al., ACS Cent. Sci. 2020, 6, 591-605. A severe global shortage of reagents in the face of unprecedented demand has hampered such testing. See, e.g., Guglielmi, 2020. Alternatives have been proposed, including the isothermal detection of target RNA (see, e.g., Emergency Use Authorization (EUA) Summary for the COLOR SARS-CoV-2 LAMP Diagnostic Assay available at www.fda.gov/media/138249/download, last accessed Aug. 10, 2020; and Abbott RealTime SARS-CoV-2 available at www.fda.gov/media/136258/download, last accessed Aug. 10, 2020) and variants of the genome-editing tool CRISPR/Cas9 for sequence detection (First Point-of-Care Test for COVID-19 Leveraging CRISPR Technology available at www.genengnews.com/virology/coronavirus/first-point-of-care-test-for-covid-19-leveraging-crispr-technology/, last accessed Aug. 10, 2020). In order to meet the need for high capacity testing, molecular diagnostic tests such as CRISPR-based detection (Cepheid Sherlock Biosciences) and isothermal amplification technologies (ID NOW COVID-19, IAMP®) have recently been granted Emergency Use Authorization (EUA) by the Food and Drug Administration (FDA). These tests are suitable for deployment in low-resource settings and can be applied widely as rapid diagnostic tests for diverse populations, particularly those with socioeconomic disadvantages. See, e.g., Hooper et al.

Loop-mediated isothermal amplification (LAMP) can detect SARS-CoV-2 RNA in a simple assay that does not require sophisticated equipment and has recently been granted emergency use authorization by the Food and Drug Administration (FDA). See., e.g., Abbott RealTime SARS-CoV-2; First Point-of-Care Test for COVID-19 Leveraging CRISPR Technology; Hooper et al.; and AQ-TOP™ COVID-19 Rapid Detection Kit available at www.fda.gov/media/138307/download, last accessed Aug. 18, 2020. LAMP allows amplification at a constant temperature and typically has a turnaround time of less than 1 hour, which offers improved screening throughput during a pandemic. See, e.g., Larremore et al., Sci Adv. 2021 Jan. 1; 7(1):eabd5393. A combination of reverse transcription and LAMP (RT-LAMP) can detect RNA samples by producing complementary DNA (cDNA) for amplification. RT-LAMP has already been used to detect influenza virus (see, e.g., Ahn et al., BMC Infect. Dis. 2019, 19, 676), Ebola virus (see, e.g., Oloniniyi et al., J. Virol. Methods 2017, 246, 8-14), and Zika virus (see, e.g., Silva et al., Sci. Rep. 2019, 9, 1-12). RT-LAMP amplification products can be detected by colorimetry (see, e.g., Ahn et al.), turbidimetry (see, e.g., Yan et al., Clin. Microbiol. Infect. 2020, 26, 773-779), fluorescence analysis (see, e.g., Lamb et al., PLoS One 2020, 15, e0234682), or gel electrophoresis (see, e.g., Baek et al., Emerg. Microbes Infect. 2020, 9, 998-1007). RT-LAMP assays can be carried out anywhere—even in the field, as long as a heat source is available. While the RT-LAMP method is a powerful tool for low-tech and rapid diagnostic testing, the development of the assay is cumbersome due to its complexity: The procedure requires four or six primers that achieve the autocycling strand displacement of oligonucleotides for amplification (see, e.g., Notomi et al., Nucleic Acids Res. 2000, 28, e63). LAMP primers, especially the inner primers, require high-performance liquid chromatography (HPLC) purification to reduce non-specific laddering on agarose gels (which is often used as secondary validation, see, e.g., Harper et al., Phytopathology 2010, 100, 1282-1288). Another limitation is that multiplex RT-LAMP for simultaneous detection of multiple targets remains challenging due to complexity of the primer design and challenges to differentiate multiple amplicons. However, newer approaches such as use of modified primers coupled with biotin tags or fluorophores enable different modes of detection for amplicon subsets using lateral-flow (see, e.g., Zhu et al., Biosens. Bioelectron. 2020, 166, 112437) or fluorescent methods (see, e.g., Zhang et al., medRxiv 2020, 2020.10.26.20219972); and thus hold promise to pave the way for multiplex RT-LAMP detection assays.

Testing is the key to confine cases and end the spread of SARS-CoV-2. The World Health Organization (WHO) advised that the positivity rate (i.e. the percent of all tests that are positive) should remain at 5% or lower for at least 2 weeks before reopening. See, e.g., Which U.S. States Meet WHO Recommended Testing Criteria? available at coronavirus.jhu.edu/testing/testing-positivity, last accessed Jul. 13, 2020. Under economic and political pressures, most localities have proceeded with erratic, phased reopenings even though the US positivity rate is about 2-fold higher as of 8 Aug. 2020. See, e.g., Testing Data in the U.S. available at www.cdc.gov/coronavirus/2019-ncov/cases-updates/testing-in-us.html, last accessed Aug. 8, 2020. Experts agree that durable containment of SARS-CoV-2 depends on more widespread testing. See, e.g., Mallapaty, Nature 2020, 167, d41586-020.

Clinical diagnostic assays for COVID infection, whether the LAMP assay (quick, one-pot), or RT-PCR assay (accurate and sensitive), use positive controls to ensure that the test is working and sensitive. Currently, convalescent plasma is used as the positive control. Such material is highly variable, not simple to obtain, and not stable.

Thus there exists a need in the art for a stable, positive control for these tests that is reliable and easy to obtain. This disclosure satisfies these needs and provides related advantages as well.

### SUMMARY OF THE DISCLOSURE

Provided herein is a synthetic ‘positive control’ for COVID assays, using a plant virus. It is easy and quick to manufacture at scale, cheap to produce, stable, and usable in a range of assays. The data appear to indicate it is precise and accurate as a control.

The COVID-19 pandemic has upended global civilization with hundreds of thousands infected and no end in sight. While treatments and vaccines are undergoing development and testing, no cure is yet available. Therefore, detection and monitoring are the first step, and currently the only option, to prevent the spread of the disease. Disclosed herein is a bioinspired nanoparticle-based platform technology that capitalizes on the unique features of the 2019-novel Coronavirus (nCoV) but is non-hazardous and safe to use in diagnostic assays. This biomimicry and biomaterials approach enables sensitive detection of nCoV with higher accuracy than available tests through the reduction of false negative results. The probe is comprised of synthetic nCoV RNA sequences (specifically the CDC-designated target sequences) that are noninfectious and replication-deficient, encapsulated into a plant virus capsid. Bioengineering design and self-assembly of the virus-based biomaterial enables the swift development and production of nCoV mimicries. The novel diagnostic probe can be used in the CDC-established qRT-PCR detection assays. It also can be used in a RT-LAMP assay that avoids the need for RNA extraction, does not require qRT-PCR instrumentation, and provides colorimetric read out within a couple of hours. These probes enable screening and testing for COVID-19, not only in human specimens, but also in livestock and agricultural samples. The development of the proposed biomaterials and detection assay could be carried out within a few months, and its production would be easily scalable to make it available to test patients in the US and worldwide. In one embodiment, the probe is specific to cowpea chlorotic mottle virus (CCMV). In another embodiment, it also can be applied to the bacteriophage Qbeta.

In one aspect, provided is a recombinant polynucleotide comprising, or alternatively consisting essentially of, or yet further consisting of (a) a first polynucleotide comprising, or alternatively consisting essentially of, or yet further consisting of a bacteriophage Qβ (QB or Qbeta) hairpin loop with affinity for the QB coat protein (CP); (b) any one or any two or all three of: (i) a second polynucleotide encoding a SARS-CoV-2 N1 or a fragment thereof; (ii) a third polynucleotide encoding a SARS-CoV-2 N2 or a fragment thereof, or (iii) a fourth polynucleotide encoding a human RNase P (RP) or a fragment thereof, and (c) an optional fifth polynucleotide encoding the QB CP.

In another aspect, provided is a recombinant polynucleotide comprising, or alternatively consisting essentially of, or yet further consisting of (a) an optional first polynucleotide comprising a bacteriophage Qβ (QB) hairpin loop with affinity for the QB coat protein (CP); (b) any one or any two or all three of: (i) a second polynucleotide encoding a SARS-CoV-2 N1 or a fragment thereof; (ii) a third polynucleotide encoding a SARS-CoV-2 N2 or a fragment thereof, (iii) a fourth polynucleotide encoding a human RNase P (RP) or a fragment thereof, and (c) a fifth polynucleotide encoding a coat protein of a cowpea chlorotic mottle virus (CCMV).

In some embodiments, the recombinant polynucleotide comprises, or alternatively consists essentially of, or yet further consists of any one or more of: SEQ ID NO: 1, SEQ ID NO: 2, a complementary sequence of each thereof, or an equivalent of each thereof (such as an RNA comprising each of the thymine (T) residues substituted with a uracil (U) residue). In some embodiments, the recombinant polynucleotide is double-stranded. In some embodiments, the recombinant polynucleotide is an RNA.

In a further aspect, provided is a vector comprising, or alternatively consisting essentially of, or yet further consisting of a polynucleotide as disclosed herein.

In yet a further aspect, provided is a system comprising, or alternatively consisting essentially of, or yet further consisting of: (1) a first vector comprising, or alternatively consisting essentially of, or yet further consisting of: (a) an optional first polynucleotide comprising, or alternatively consisting essentially of, or yet further consisting of a bacteriophage Qβ (QB) hairpin loop with affinity for the QB coat protein (CP), or a polynucleotide complementary thereto, and (b) any one or any two or all three of: (i) a second polynucleotide encoding a SARS-CoV-2 N1 or a fragment thereof or a polynucleotide complementary thereto, (ii) a third polynucleotide encoding a SARS-CoV-2 N2 or a fragment thereof or a polynucleotide complementary thereto, or (iii) a fourth polynucleotide encoding a human RNase P (RP) or a fragment thereof or a polynucleotide complementary thereto; and (2) a second vector comprising, or alternatively consisting essentially of, or yet further consisting of: (c) a fifth polynucleotide encoding a coat protein or a polynucleotide complementary thereto. In some embodiments, the first vector comprises, or alternatively consists essentially of, or yet further consists of any one or more of: SEQ ID NO: 1, SEQ ID NO: 2, a complementary sequence of each thereof, or an equivalent of each thereof. In some embodiments, the polynucleotide of (b) in the first vector is out-of-frame and does not express any one of: an N1, an N2, an RP, or a fragment of each thereof. Additionally or alternatively, the first vector does not comprise (i.e., lacks) a ribosome binding site (RBS) upstream to the polynucleotide of (b).

In one aspect, provided is a host cell comprising one or more of: a polynucleotide as disclosed herein, a vector as disclosed herein, or a system as disclosed herein. In some embodiments, the cell is a prokaryotic cell, optionally an Escherichia coli (E. coli), and further optionally a BL21 (DE3) E. coli cell.

In a further aspect, provided is a method of preparing a recombinant polynucleotide as disclosed herein, such as an RNA polynucleotide as disclosed herein. The method comprises, or alternatively consists essentially of, or yet further consists of growing a host cell as disclosed herein under conditions to replicate or express the polynucleotide.

Alternatively, provided is a method of preparing a cowpea chlorotic mottle virus (CCMV) comprising, or alternatively consisting essentially of, or yet further consisting of a CCMV coat protein and a polynucleotide as disclosed herein (such as an RNA polynucleotide as disclosed herein). The method comprises, or alternatively consists essentially of, or yet further consists of mixing the polynucleotide with a CCMV coat protein in vitro.

In one aspect, provided is a virus-like particle (VLP) comprising, or alternatively consisting essentially of, or yet further consisting of a polynucleotide encapsidated in a coat protein (CP). In some embodiments, the polynucleotide is an RNA polynucleotide. In further embodiments, the RNA polynucleotide is disclosed herein. Additionally or alternatively, the RNA polynucleotide comprises, or alternatively consists essentially of, or yet further consists of (a) an optional first polynucleotide comprising, or alternatively consisting essentially of, or yet further consisting of a bacteriophage Qβ (QB) hairpin loop with affinity for the QB coat protein (CP), or a polynucleotide complementary thereto, and (b) any one or any two or all three of: (i) a second polynucleotide encoding a SARS-CoV-2 N1 or a fragment thereof or a polynucleotide complementary thereto, (ii) a third polynucleotide encoding a SARS-CoV-2 N2 or a fragment thereof or a polynucleotide complementary thereto, or (iii) a fourth polynucleotide encoding a human RNase P (RP) or a fragment thereof or a polynucleotide complementary thereto. In some embodiments, the VLP is derived from a QB and the CP comprises, or alternatively consists essentially of, or yet further consists of a QB coat protein. In some embodiments, the VLP is derived from a CCMV and the CP comprises, or alternatively consists essentially of, or yet further consists of a CCMV coat protein.

In a further aspect, provided is a method of preparing a VLP as disclosed herein. The method comprises, or alternatively consists essentially of, or yet further consists of culturing a host cell as disclosed herein under conditions suitable for producing the VLP. In some embodiments, the method further comprises isolating the VLP. In some embodiments, the VLP is derived from QP.

In one aspect, provided is a method for detecting a SARS-CoV-2 infection in a subject in need thereof. The method comprises, or alternatively consists essentially of, or yet further consists of (1) contacting a sample suspected of comprising, or alternatively consisting essentially of, or yet further consisting of the SARS-CoV-2 and isolated from the subject with a probe that generates a detectable signal when contacting with any one or any two or all three of: (i) a second polynucleotide encoding a SARS-CoV-2 N1 or a fragment thereof, (ii) a third polynucleotide encoding a SARS-CoV-2 N2 or a fragment thereof, or (iii) a fourth polynucleotide encoding a human RNase P (RP) or a fragment thereof, (2) separately contacting a VLP as disclosed herein with the probe; and (3) measuring the detectable signal of the contacted sample of (1) and the detectable signal of the contacted VLP of (2). In some embodiments, the measured signal of the contacted sample of (1) at a level substantially similar or higher than the measured signal of the contacted VLP of (2) indicates the sample comprises, or alternatively consists essentially of, or yet further consists of SAR-CoV-2. In further embodiments, the measured signal of the contacted sample of (1) at a level substantially similar or higher than the measured signal of the contacted VLP of (2) indicates the subject has a SARS-CoV-2 infection. In some embodiments, the method comprises, or alternatively consists essentially of, or yet further consists of a reverse transcription polymerase chain reaction (RT-PCR), optionally a quantitative RT-PCR (qRT-PCR) or a droplet digital PCR (ddPCR). Additionally or alternatively, the method comprises, or alternatively consists essentially of, or yet further comprises of a reverse transcription loop-mediated isothermal amplification (RT-LAMP), optionally a swab-to-test RT-LAMP assay.

Additionally, provided is a kit comprising, or alternatively consisting essentially of, or yet further consisting of instructions for use and any one or more of: a polynucleotide as disclosed herein, a vector as disclosed herein, a system as disclosed herein, a host cell as disclosed herein, or a VLP as disclosed herein. In further embodiments, the kit is for use in a method as disclosed herein.

### BRIEF DESCRIPTION OF THE FIGURES

FIGS. 1A-1C provide an overall schematic and workflow of the virus-like particle (VLP) based biomimetic SARS-CoV-2 positive controls. FIG. 1A shows a design of a SARS-CoV-2 Detection Module (SDM) from 5′ to 3′: T7 promoter, Qβ hairpin, N1, N2, RP, and T7 terminator, each of which is marked. FIG. 1B shows production of Qβ 1P-C19 and Qβ 2P-C19 VLPs via in vivo assembly. Qβ 1P-C19 VLPs were produced from a one-plasmid (1P) system, where the gene of the Qβ coat proteins (CPs) and SDM RNAs were cloned in one vector (pCDFDuet™-1). Qβ 2P-C19 VLPs were produced from a two-plasmid (2P) system, where Qβ CP was derived from pCDFDuet-Qβ and SDM RNA was produced from pET-28a (+). FIG. 1C shows that CCMV-C19 VLPs were produced by in vitro reconstitution of the transcribed SDM RNAs with purified CCMV CPs.

FIGS. 2A-2D provide characterization of VLP-based SARS-CoV-2 positive controls. Agarose gel results are provided in FIG. 2A: Native agarose gel electrophoresis of VLPs packaging the SDM with gels were stained with GELRED™ Nucleic Acid Stain (RNA stain), and Coomassie blue (protein stain) to show the presence of RNAs and VLPs. DLS results are provided FIG. 2B: Dynamic light scattering (DLS) of VLPs packaging the SDM; triplicate samples were analyzed, and representative data sets are shown. TEM results are provided in FIG. 2C: Imaging of negatively stained VLPs packaging the SDM using transmission electron microscope (TEM). Average size of 20 particles tabulated by ImageJ software is stated in the inset box. SEC results are provided in FIG. 2D: Analysis of positive controls by size exclusion chromatography (SEC) using a Superose 6 column and GE Healthcare Äkta Purifier chromatography system; protein was detected at 280 nm and RNA was detected at 260 nm.

FIG. 3 shows that a SARS-CoV-2 detection module (SDM) is flanked by a T7 promoter and T7 terminator at the 5′ and 3′ ends, respectively, for ribonucleic acid (RNA) transcription. The sequence as shown is reproduced in SEQ ID NO: 1. T7 promoter and T7 terminator are at the 5′ terminus and 3′ terminus, respectively, while their sequences are represented using italicized letters. The Qβ hairpin sequence is underlined. SARS-CoV-2 N1 region immediately follows the Qβ hairpin sequence while its sequence is represented using italicized and bold letters. SARS-CoV-2 N2 region immediately follows the N1 region while its sequence is represented using bold and underlined letters. RP region are represented using bold letters. The SDM flanked with a T7 promoter and T7 terminator (SDM+T7_{P/T}) was cloned not in frame with open reading frame of plasmid to avoid proper protein translation of a truncated SARS-CoV-2 gene. N represents SARS-CoV-2 nucleocapsid gene; RP represents human RNase P gene.

FIGS. 4A-4B provide vector maps of plasmids for production of Qβ 1P-C19 VLPs and Qβ 2P-C19 VLPs. FIG. 4A shows a one-plasmid system for production of Qβ 1P-C19 VLPs: SDM+T7_{P/T} was cloned downstream of Qβ capsid protein in plasmid pCDFDuet-Qβ to construct plasmid Qβ 1P-C19. FIG. 4B shows a two plasmids system for production of QP 2P-C19 VLPs: SDM+T7_{P/T} was cloned in pET-28a (+) to construct plasmid Qβ 2P-C19 and co-express Qβ capsid protein (CP) from pCDFDuet™-Qβ. N1, N2, and RP in SDM were labelled.

FIGS. 5A-5C provide characterization of Qβ VLP by DLS (FIG. 5A), FPLC (fast protein liquid chromatography or SEC, FIG. 5B), and TEM (FIG. 5C).

FIGS. 6A-6D provide characterization of wild type (WT) CCMV by DLS (FIG. 6A), FPLC (fast protein liquid chromatography or SEC, FIG. 6C), TEM (FIG. 6B), and agarose gel (FIG. 6D). Disassembled CCMV was analyzed by agarose gel shown in FIG. 6D. Lane 1: WT CCMV. Lane 2: Disassembled CCMV. The same gel was stained by GELRED™ and Coomassie blue to show the presence/absence of RNA.

FIGS. 7A-7C provide characterization of the in vitro transcribed SDM RNA. FIG. 7A provides an analysis of in vitro transcribed SDM RNA on a denaturing 6% urea polyacrylamide gel. M: Thermo Fisher Scientific CENTURY™-Plus RNA Markers. Lane 1: 200 ng of SDM RNA. FIG. 7B provides determination of purity of SDM RNA by Nanodrop. Ratio 260/280 and 260/230 were determined. FIG. 7C provides an Quantitative reverse transcription polymerase chain reaction (RT-qPCR) result of in vitro transcribed SDM RNA by United States' The Centers for Disease Control and Prevention (US CDC) primers/probes sets for all the three regions (N1, N2, RP). No template control (NTC) serves as negative control.

FIGS. 8A-8B provide an analysis of the total nucleic acids extracted from VLPs. FIG. 8A shows that total nucleic acids extracted from VLPs were analyzed on 6% denaturing urea polyacrylamide gel. M: Thermo Fisher Scientific CENTURY™-Plus RNA Markers. Lane 1: Qβ VLP. Lane 2: Qβ 1P-C19. Lane 3: Qβ 2P-C19. Lane 4: CCMV-C19. Arrow indicates the SDM RNA. A total of 150 ng RNA was loaded into each well. FIG. 8B provides equations for normalization of copy number.

FIGS. 9A-9B show release kinetics of encapsidated SDM RNAs from VLPs at 75° C. at various time points as determined by agarose gel electrophoresis (FIGS. 9A-9B) and RT-qPCR (FIGS. 9C-9D). VLPs were analyzed by native agarose gel electrophoresis after heating at 75° C. for 0 min (Lane 1), 0.5 min (Lane 2), 1 min (Lane 3), 2 min (Lane 4), 3 min (Lane 5), 4 min (Lane 6), and 5 min (Lane 7) for Qβ 1P-C19 and Qβ 2P-C19. At time point 0 (Lane 1), SDM is encapsidated and RNA and proteins co-migrate (*); 0.5 min exposure at 75° C. results in SDM release (*); the amount of released SDM increased over the 5 min time course. CCMV-C19 was heated at 75° C. for 0 min (Lane 1), 1 min (Lane 2), 5 min (Lane 3), 10 min (Lane 4), 20 min (Lane 5), 30 min (Lane 6), and 60 min (Lane 7). Released SDM is indicated by the asterisk. Gels indicate disassembly or denaturing of the CCMV assembly with RNA and protein observed in the pockets of the gel. Same gels were stained with GELRED™ Nucleic Acid Stain (RNA stain, FIG. 9A), and Coomassie blue (protein stain, FIG. 9B). RNA bands excised from gels for RT-qPCR were labelled by asterisks for Qβ 1P-C19 and Qβ 2P-C19 as well as for CCMV-C19. RNA band intensities (FIG. 9C) from 30 s (Qβ 1P-C19 and Qβ 2P-C19) and 1 min (CCMV-C19) were evaluated by ImageJ software. As shown in FIG. 9D, RT-qPCR was performed using all three sets of primer/probe sets (N1, N2, RP). NTC represents no-template control in RT-qPCR.

FIGS. 10A-10C provide validation of CDC primer/probe sets on in vitro transcribed SDM RNAs. RT-qPCR was performed on in vitro transcribed SDM RNAs (10⁶-10⁰ copies) to construct N1 (FIG. 10A), N2 (FIG. 10B), and RP (FIG. 10C) standard curves for tabulating PCR amplification efficiency. Error bars show the standard deviation.

FIGS. 11A-11C provide validation of VLP-based SARS-CoV-2 positive controls in the clinical setting using droplet digital PCR (ddPCR) detection of SARS-CoV-2. FIG. 11A provides ddPCR 1-D amplitude plots of SARS-CoV-2 positive controls according to N1, N2, and RP regions. Lane 1: Qβ 1P-C19. Lane 2: Qβ 2P-C19. Lane 3: CCMV-C19. Lane 4: (+) Clinical sample from COVID-19 confirmed patient. Lane 5: (−) Clinical sample from healthy patient for N1 and N2 (negative control); no template control for RP. Amplifications were performed in triplicate. FIG. 11B provides a scatter plot comparing copy numbers of SARS-CoV-2 detection regions (N1, N2, RP) for all positive controls. FIG. 11C provides tabulated SDM RNA copy number for each SARS-CoV-2 positive controls.

FIGS. 12A-12D provide characterization of VLP-based SARS-CoV-2 positive controls after 1-month storage under ambient conditions. Agarose gel results are provided in FIG. 12A: Native agarose gel electrophoresis of VLP-based SARS-CoV-2 positive controls reveals intact VLPs; gels were stained with GELRED™ Nucleic Acid Stain (RNA stain), and Coomassie blue (protein stain) to show the presence of RNAs and VLPs. DLS results are provided in FIG. 12B: Dynamic light scattering (DLS) of VLPs packaging the SDM; triplicate samples were analyzed, and representative data sets are shown. TEM results are provided in FIG. 12C: Imaging of negatively stained VLPs packaging the SDM using transmission electron microscope (TEM). Average size of 20 particles tabulated by ImageJ software is stated in the inset box. SEC results are provided in FIG. 12D: Analysis of positive controls by size exclusion chromatography (SEC) using a Superose 6 column and GE Healthcare Äkta Purifier chromatography system; protein was detected at 280 nm and RNA was detected at 260 nm. See also FIG. 2; showing the characterization of freshly-prepared samples.

FIG. 13 shows stability of VLP-based SARS-CoV-2 positive controls in respect to time and temperature. RT-qPCR was performed to obtain the Cq values. Triplicates were performed on each sample with the error bar showing the standard deviation.

FIG. 14 provides a graphic summary of the disclosure.

FIGS. 15A-15C provides an overall schematic and workflow of the VLP-based biomimetic SARS-CoV-2 positive controls, i.e., construction of the SLDM positive controls.

FIG. 15A shows a design of SLDM from 5′ to 3′: T7 promoter, Qβ hairpin, N detection region, restriction sites, RP detection region, and T7 terminator (grey), each of which is labelled. The N and RP segments contain both RT-qPCR and RT-LAMP detection regions. FIG. 15B shows production of Qβ 1P-C19L and Qβ 2P-C19L VLPs via in vivo assembly. Qβ 1P-C19L: VLPs were produced using a one-plasmid system, in which the SLDM and Qβ coat protein genes were present on the same vector (pCDFDuet-1). Qβ 2P-C19L: VLPs were produced using a two-plasmid system, in which SLDM RNA was produced from pET-28a (+), and Qβ coat protein was produced from pCDFDuet-Qβ. FIG. 15C shows that CCMV-C19L VLPs were produced by the reassembly of in vitro transcribed SLDM RNAs and purified CCMV coat proteins.

FIG. 16 provides that a SARS-CoV-2 LAMP detection module (SLDM) is flanked by a T7 promoter and T7 terminator at the 5′ and 3′ ends, respectively, for in vitro transcription. The sequence as shown is reproduced in SEQ ID NO: 2. T7 promoter and T7 terminator are at the 5′ terminus and 3′ terminus, respectively, while their sequences are represented using italicized letters. The Qβ hairpin is underlined. SARS-CoV-2 N regions are indicated using italicized and bold letters with (N1) or without (N2) underlines. RP region is indicated using black and bold letters. RT-LAMP detection regions are GTAACACAAG CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC ATTGGCATGG AAGTCACACC TTCGGGAACG TGGTTGACCT ACACAGGTGC CATCAAATTG GATGACAAAG ATCCAAATTT CAAAGATCAA (i.e., nt 226 to nt 465 of SEQ ID NO: 2) and CTTGTTGATG AGCTGGAGCC AGAGACCGAC ACACGGGAGC CACTGACTCG GATCCGCAAC AACTCAGCCA TCCACATCCG AGTCTTCAGG GTCACACCCA AGTAATTGAA AAGACACTCC TCCACTTATC CCCTCCGTGA TATGGCTCTT CGCATGCTGA GTACTGGACC TCGGACCAGA GCCATGTAAG AAAAGGCCTG TTCCCTGGAA GCCCAAAGGA CTCTGCATTG AGGGTG (i.e., nt 472 to nt 707 of SEQ ID NO: 2). Restriction sites Pst1 and SaI1 (CTGCAG, i.e., nt 220 to nt 225 of SEQ ID NO: 2, or GTCGAC, i.e., nt 466 to nt 471 of SEQ ID NO: 2) were placed between genes to facilitate sub-cloning. Further restriction sites (Qβ 1P-C19L: Not1, Nde1; Qβ 2P-C19L: BgIII, Not1) were placed before the T7 promoter and after the T7 terminator. The SLDM with flanking T7 promoter and T7 terminator (SLDM+T7_{P/T}) was cloned out-of-frame in the plasmid to avoid translation of the truncated SARS-CoV-2 N gene.

FIGS. 17A-17B provide vector maps of plasmids used for the production of Qβ 1P-C19L VLPs and Qβ 2P-C19L VLPs. FIG. 17A shows a one-plasmid system for the production of Qβ 1P-C19L VLPs: SLDM+T7_{P/T} was inserted downstream of the Qβ coat protein (CP) gene in pCDFDuet-Qβ. FIG. 17B shows a two-plasmid system for the production of Qβ 2P-C19L VLPs: the SLDM+T7_{P/T} was cloned in pET-28a (+) to generate plasmid Qβ 2P-C19L and was co-expressed with the Qβ coat protein from pCDFDuet-Qβ. The N and RP regions of the SLDM are labelled as shown.

FIGS. 18A-18B show construction of the SARS-CoV-2 LAMP detection module (SLDM) by assembly PCR. FIG. 18A shows the overall workflow of assembly PCR. The SLDM was constructed by joining three fragments containing the detection regions (N, RP), QP hairpin, T7 promoter and T7 terminator. FIG. 18B shows an agarose gel electrophoresis of the three fragments (lanes 1, 2 and 3) and the full SLDM constructs in pCDFDuet-Qβ(lane 4) and pET-28a(+) (lane 5).

FIGS. 19A-19D provide characterization of in vitro transcribed SLDM RNA. FIG. 19A provides an analysis of SLDM RNA by denaturing 6% urea polyacrylamide gel electrophoresis. M=Century Plus RNA markers; Lane 1=200 ng of SLDM RNA. FIG. 19B shows purity of SLDM RNA (260/280 and 260/230 nm absorbance ratios). As shown in FIG. 19C, RT-qPCR was used to detect the N and RP regions with corresponding no template controls (NTC). FIG. 19D shows triplicate RT-LAMP assays using N and RP primer sets.

FIGS. 20A-20D provide validation of primer/probe sets using in vitro transcribed SLDM RNAs. RT-qPCR was performed on in vitro transcribed SLDM RNAs (10⁶-10⁰ copies) to construct standard curves for the N (FIG. 20A) and RP regions (FIG. 20B) to tabulate PCR amplification efficiency. Error bars show the standard deviation. As shown in FIG. 20C, RT-LAMP was performed on in vitro transcribed SLDM RNAs (10⁶-10⁰ copies) to determine the detection limits for N and RP using RT-LAMP primers. As shown in FIG. 20D, Images of the RT-LAMP reactions were captured following incubation at 65° C. for 55 min to show the color change. A no template control (NTC) was included as a negative control. Numbers on tubes represent powers of 10 in terms of SLDM RNA copies (e.g., 6=10⁶ copies).

FIGS. 21A-21D provide characterization of VLP-based SLDM positive controls. FIG. 21A shows the analysis of VLPs by native agarose gel electrophoresis followed by staining with GELRED™ (nucleic acid stain) and Coomassie Brilliant Blue (protein stain), confirming the particles are intact: lane 1=Qβ VLPs; lane 2=Qβ 1P-C19L; lane 3=Qβ 2P-C19L; lane 4=wild type CCMV; lane 5=CCMV-C19L. FIG. 21B shows the analysis of VLPs by dynamic light scattering (DLS). Triplicate samples were analyzed, and representative datasets are shown. PDI represents polydispersity index. FIG. 21C shows negatively-stained VLPs analyzed by transmission electron microscopy (TEM). The average size of 20 particles tabulated by ImageJ software is presented below each image. FIG. 21D shows the analysis of VLPs by size exclusion chromatography (SEC) using a Superose 6 column and GE Healthcare Äkta Purifier chromatography system. Nucleic acids were detected at 260 nm and protein was detected at 280 nm.

FIG. 22 shows that total nucleic acids extracted from VLPs was analyzed by denaturing 6% urea polyacrylamide gel electrophoresis (150 ng RNA per lane). M=Century-Plus RNA markers; lane 1=Qβ VLP; lane 2=Qβ 1P-C19L; lane 3=Qβ 2P-C19L; lane 4=CCMV-C19L. Arrow indicates the SLDM RNA.

FIGS. 23A-23B show batch-to-batch consistency of the SLDM-loaded VLPs. FIG. 23A provides a scatter plot comparing Cq values of SLDM RNAs extracted from three batches each of Qβ 1P-C19L, Qβ 2P-C19L, and CCMV-C19L, respectively. Triplicate experiments were performed for each batch, and mean with standard deviation was plotted. Statistical significance between batches was determined by one-way analysis of variance (ANOVA) using GraphPad Prism. FIG. 23B provides a table showing mean Cq for each batch of SLDM RNAs extracted from VLPs. SD=standard deviation of Cq for the three batches.

FIGS. 24A-24B provide a comparison of stability of free and VLP-encapsidated SLDM RNAs. FIG. 24A shows results of a temperature/time incubation test. Stability of free SLDM RNAs and VLP encapsidated SLDM RNAs in respect to time and temperature was plotted against Cq values. Triplicates were performed on each sample with the error bar showing the standard deviation. FIG. 24B shows results of an RNase A digestion test: Free SLDM RNAs were analyzed with denaturing urea polyacrylamide gel, and all VLP-based positive controls were analyzed with native agarose gel electrophoresis. (M) Century-Plus RNA markers. (Lane 1) Fresh free SLDM RNA. (Lane 2) Free SLDM RNA incubated at 37° C. (Lane 3) Free SLDM RNA incubated at 37° C. with 25 μg RNase A. (Lane 4) Qβ 1P-C19L incubated at 37° C. (Lane 5) Qβ 1P-C19L incubated at 37° C. with 25 μg RNase A. (Lane 6) Qβ 2P-C19L incubated at 37° C. (Lane 7) Qβ 2P-C19L incubated at 37° C. with 25 μg RNase A. (Lane 8) CCMV-C19L incubated at 37° C. (Lane 9) CCMV-C19L incubated at 37° C. with 25 μg RNase A.

FIGS. 25A-25C show serial dilutions of total nucleic acids extracted from VLPs analyzed by RT-qPCR (FIG. 25A) and RT-LAMP (FIGS. 25B-25C). The extracts were analyzed to detect the presence of SLDM RNAs. Error bars depict standard deviations. In the RT-LAMP assay and as shown in FIG. 25B, the dotted line at 0.3 on the y-axis separates the positive and negative clusters. As shown in FIG. 25C, images indicate the color change in the RT-LAMP assay and are arranged according to the x-axis in the RT-qPCR panel (NTC=no template control).

FIGS. 26A-26E show release of RNA from VLPs. VLPs before heating (t0) and after 5 min at 95° C. (t5) were analyzed by native agarose gel electrophoresis (FIGS. 26A-26B). The RNA and protein in the gels were stained with GELRED™ (FIG. 26A) and Coomassie Brilliant Blue (FIG. 26B), respectively. The RNA bands were excised and analyzed by RT-qPCR (FIG. 26C) and RT-LAMP electrophoresis (FIGS. 26D-26E) to confirm the presence of SLDM RNAs. Error bars depict standard deviations. In the RT-LAMP assay and as shown in FIG. 26D, the dotted line at 0.3 on the y-axis separates the positive and negative clusters. As shown in FIG. 26E, images indicate the color change in the RT-LAMP assay, with N(−) and RP(−) representing the no template control.

FIGS. 27A-27C show serial dilutions of VLPs heated to 95° C. for 5 min to release encapsidated RNAs. The total RNA was analyzed by RT-qPCR (FIG. 27A) and RT-LAMP (FIGS. 27B-27C) to detect the presence of SLDM RNA. Error bars depict standard deviations. In the RT-LAMP assay and as shown in FIG. 27B, the dotted line at 0.3 on the y-axis separates the positive and negative clusters. As shown in FIG. 27C, images indicate the color change in the RT-LAMP assay and are arranged according to the x-axis in the RT-qPCR panel (NTC=no template control).

FIGS. 28A-28E provide validation of SLDM positive controls in clinical settings. FIG. 28A provides droplet digital PCR one-dimensional amplitude plots of SARS-CoV-2 positive controls (N and RP regions): lane 1=Qβ 1P-C19L; lane 2=Qβ 2P-C19L; lane 3=CCMV-C19L; lane (+)=COVID-19 patient sample; lane 5 (N)=healthy patient sample (negative control for N); lane 5 (RP)=no template control (negative control for RP). Data represent triplicate amplifications. The horizontal line is the cutoff between positive clusters and negative clusters. FIG. 28B provides a scatter plot comparing N and RP copy numbers for all positive controls. The bars indicate median values. FIG. 28C provides SLDM RNA copy numbers detected for every microgram of SARS-CoV-2 positive controls. FIG. 28D provides results of a swab-to-test assay of SLDM positive controls with clinical samples in duplicate. The dotted line at 0.3 on the y-axis separates the positive and negative clusters (POS=COVID-19 patient sample, NEG=healthy patient sample, NTC=no template control). The bars indicate median values. FIG. 28E provides a representative image of the swab-to-test reaction showing the color change from negative to positive.

FIG. 29 provides a graphic summary relating to the SARS-CoV-2 LAMP detection module.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

(As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

In some embodiments, the terms “first” “second” “third” “fourth” or similar in a component name are used to distinguish and identify more than one components sharing certain identity in their names. For example, “first polynucleotide” and “second polynucleotide” are used to distinguishing two polynucleotides.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, or protein, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, or protein, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the phrase “derived from” means isolated from, purified from, or engineered from, or any combination thereof.

As used herein, “complementary” sequences refer to two nucleotide sequences which, when aligned anti-parallel to each other, contain multiple individual nucleotide bases which pair with each other. Accordingly, the two nucleotide sequences are complementary to each other or a complement of each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, the identity is calculated between two peptides or polynucleotides over their full-length, or over the shorter sequence of the two, or over the longer sequence of the two.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: blast.ncbi.nlm.nih.gov/Blast.cgi, last accessed on Aug. 1, 2021. In another embodiment, the program is any one of: Clustal Omega accessible at www.ebi.ac.uk/Tools/msa/clustalo/, Needle (EMBOSS) accessible at www.ebi.ac.uk/Tools/psa/emboss_needle/, Stretcher (EMBOSS) accessible at www.ebi.ac.uk/Tools/psa/emboss_stretcher/, Water (EMBOSS) accessible at www.ebi.ac.uk/Tools/psa/emboss_water/, Matcher (EMBOSS) accessible at www.ebi.ac.uk/Tools/psa/emboss_matcher/, LALIGN accessible at www.ebi.ac.uk/Tools/psa/lalign/. In further embodiments, the default setting is used.

In some embodiments, the polynucleotide as disclosed herein is a RNA or an analog thereof. In some embodiments, the polynucleotide as disclosed herein is a DNA or an analog thereof. In some embodiments, the polynucleotide as disclosed herein is a hybrid of DNA and RNA or an analog thereof.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complementary. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” refers to the process whereby a foreign nucleotide sequence is introduced into a cell. In some embodiments, this transduction is done via a vector, viral or non-viral.

As used herein, the term “probe” intends a polynucleotide, an antibody or other agent that is used to detect and identify COVID-19 coronavirus (i.e., SARS-CoV-2) in a sample.

In some embodiments, a “probe” when used in the context of polynucleotide manipulation refers to an oligonucleotide that is provided as a reagent to detect a target potentially present in a sample of interest by hybridizing with the target. Usually, a probe comprises a detectable label or marker or a means by which a label or marker can be attached, either before or subsequent to the hybridization reaction. In some embodiments, a probe as used herein is a TAQMAN© probe. For example, a probe comprises, or consists essentially of, or yet further consists of a polynucleotide which is capable of hybridizing with the target, a fluorescence reporter at either of the 5′ end of the polynucleotide or the 3′ end of the polynucleotide, and a quencher at the other end of the polynucleotide. Accordingly, the probe hybrids to its target without producing any fluorescent signal since it quencher is in a close proximity to the reporter; and upon replicating the target by a polymerase having a 5′-3′ exonuclease activity (such as a Taq polymerase), the probe is cleaved by the polymerase, releasing the fluorescence report from its quencher and accordingly producing a fluorescent signal. In further embodiments, two or more primers are required for replication of the target, such as a forward primer that is capable of hybridizing to the target downstream to the probe hybridization region, and a reverse primer that is capable of hybridizing to the complementary polynucleotide of the target and that aligned to the target upstream to the probe hybridization region. Accordingly, such primers and probe are referred to herein as a primer/probe set. Non-limiting examples of a primer/probe set can be found in Table 5. Alternatively, a “probe” can be a biological compound such as a polypeptide, antibody, or fragments thereof that is capable of binding to the target potentially present in a sample of interest.

In other embodiments, a “probe” as used herein refers to a polynucleotide used in parallel when detecting and identifying SARS-CoV-2 in a sample and serving as a control, such as a positive control comprising, or consisting essentially of, or yet further consisting of a target to be detected or a fragment thereof. In further embodiments, a probe hybridizes to a primer. Accordingly, it is also referred to herein as a control probe or a positive control probe. In some embodiments, the probe is suitable for use in polymerase chain reaction (PCT), Real-time PCR (RT-PCR), Loop Mediated Isothermal Amplification (LAMP) (see, Thi et al. Science Translational Medicine, Vol. 12, Issue 556, eabc7075), and reverse transcription LAMP (RT-LAMP). Additional assays for COVID detection are provided in Table 1.

Loop-mediated isothermal amplification (LAMP) uses one or more, such as 4-6, primers recognizing, for example 6-8, distinct regions of target DNA for a highly specific amplification reaction. A strand-displacing DNA polymerase initiates synthesis and 2 specially designed primers form “loop” structures to facilitate subsequent rounds of amplification through extension on the loops and additional annealing of primers. DNA products are very long (>20 kb) and formed from numerous repeats of the short (80-250 bp) target sequence, connected with single-stranded loop regions in long concatamers. These products are not typically appropriate for downstream manipulation, but target amplification is so extensive that numerous modes of detection are possible.

Real-time fluorescence detection using intercalators or probes, lateral flow and agarose gel detection are all directly compatible with LAMP reactions. Instrumentation for LAMP typically requires consistent heating to the desired reaction temperature and, where needed, real-time fluorescence for quantitative measurements. Optimized settings for running LAMP experiments on isothermal instruments such as the Axxin T8-ISO and T16-ISO can be found, for example, in Tanner, Optimized Integration of New England Biolabs® Loop-mediated Isothermal Amplification (LAMP) Reagents with Axxin ISO Instruments, application note published by New England Biolabs, Inc. in 2019.

In addition to the more traditional or complex detection methods, LAMP is so prolific that the products and byproducts of these reactions can also be visualized by eye. For example, magnesium pyrophosphate produced during the reaction can be observed as a white precipitate or added indicators like calcein or hydroxynaphthol blue can be used to signal a positive reaction. Alternatively, using the WarmStart® 2× Colorimetric LAMP Master Mix developed by NEB enables a strong color change from pink to yellow based on a pH change during the reaction. An updated version of this product has been formulated with dUTP and UDG to be compatible with carryover prevention between amplification rounds—WarmStart Colorimetric LAMP 2× Master Mix with UDG. The colorimetric detection technology is a key component of the SARS-CoV-2 Rapid Colorimetric LAMP Assay Kit, which can be used in the analysis of SARS-CoV-2, the novel coronavirus that causes COVID-19. Designing LAMP primers can be challenging, but software tools greatly facilitate this process, such as the NEB LAMP Primer Design Tool to design LAMP primers. After inputting a DNA or RNA sequence of interest, the LAMP Primer Design tool will identify suitable target regions and create the outer F3/B3 and looping inner FIP/BIP primers in a single step. The LoopF/LoopB primers, that accelerate the LAMP reaction, are created in a second step.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

In some embodiments, a fragment of a protein is at least about 3 amino acid (aa) long, or at least about 4 aa long, or at least about 5 aa long, or at least about 6 aa long, or at least about 7 aa long, or at least about 8 aa long, or at least about 9 aa long, or at least about 10, aa long, or at least about 15, aa long, or at least about 20 aa long, or at least about 25 aa long, or at least about 30 aa long, or at least about 35 aa long, or at least about 40 aa long, or at least about 50 aa long, or at least about 60 aa long, or at least about 70 aa long, or at least about 80 aa long, or at least about 90 aa long, or at least about 100 aa long, or at least about 120 aa long, or at least about 150 aa long, or at least about 200, or longer.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST. In one aspect, equivalent polynucleotides, proteins and corresponding sequences can be determined using BLAST (accessible at blast.ncbi.nlm.nih.gov/Blast.cgi, last accessed on Aug. 1, 2021).

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

The term “RNA” as used herein refers to its generally accepted meaning in the art. Generally, the term RNA refers to a polynucleotide comprising at least one ribofuranoside moiety. The term can include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, for example at one or more nucleotides of the RNA. Nucleotides in the nucleic acid molecules can also comprise non-standard nucleotides, such as non-naturally occurring nucleotides or chemically synthesized nucleotides or deoxynucleotides. These altered RNAs can be referred to as analogs or analogs of naturally-occurring RNA. In some embodiments, the RNA is a messenger RNA (mRNA).

A “composition” is intended to mean a combination of active agent and another compound or composition, inert (for example, a detectable agent or label) or active, such as an adjuvant, diluent, binder, stabilizer, buffers, salts, lipophilic solvents, preservative, or the like and include pharmaceutically acceptable carriers. A combination as used herein intends that the individual active ingredients of the compositions are separately formulated for use in combination, and can be separately packaged with or without specific dosages. The active ingredients of the combination can be administered concurrently or sequentially.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “virus-like particle” (VLP) refers to a structure that in at least one attribute resembles a virus, optionally which had not been demonstrated to be infections. In some embodiments, a VLP refers to a viral capsid and a polynucleotide encapsidated in the capsid. In further embodiments, the VLP is non-replicating. In yet further embodiments, the VLP lacks all or part of the viral genome, for example, the replicative and infectious components of the viral genome. Additionally or alternatively, the VLP does not infect a subject as disclosed herein, such as an animal. In some embodiments, the VLP only infects plants.

The terms “coat protein” and “viral coat protein” are used interchangeably, and refer to a protein, at least a portion of which is present on the surface of a viral particle, such as a bacteriophage Qβ or a cowpea chlorotic mottle virus. In some embodiments, a coat protein refers to a protein which create the tightly assembled structure of the protective shell (also referred to as a capsid) for a virus and prevent degradation of the viral genome, such as by environmental factors.

The term “encapsidation” generically refers to the process of incorporating a polynucleotide (e.g., a viral genome) into a viral particle. In some embodiments, encapsidation or a grammatical variation thereof refers to the process of placing a polynucleotide (e.g., single-stranded RNA, double-stranded RNA, single stranded DNA and double-stranded DNA) into any type of viral particle.

As used herein, “capsid” is a generic term used to indicate any type of viral shell, particle or coat, including a protein capsid, a lipid enveloped structure, a protein-nucleic acid capsid, or a combination thereof (e.g., a lipid-protein envelope surrounding a protein-nucleic acid capsid).

Bacteriophage Qβ (QB) is a member of the levivirida family. It is a small virus that is about 25 nm thick and is a coliphage with an RNA that is 4217 nucleotides long. As described in biology.kenyon.edu/BMB/jsmol2019/EAIJ/NewVersion81.html#:˜:text=Bacteriophage %20 QB%20is%20a%20member,%2C%20%26%20Finn%2C%202009).&text=Members%20of% 20the%20leviviridae%20family,et%20at.%2C%202018) last accessed on Aug. 17, 2021, QB has 20 faces each composed of six subunits and 12 vertices each composed of 5 subunits. Members of the leviviridae family form icosahedral capsids from 180 coat protein subunits around a 4.2 kb sense-strand RNA genome. Each of these coat proteins (capsomers) has about 132 residues of amino acids. Bacteriophage QB is a positive strand RNA virus. Positive strand RNA viruses have genomes that are functional mRNAs. For instance, QB's genome codes for 4 proteins: A1, A2, CP and QB replicase. QB has other proteins like the B-subunit of a replicase, the maturation protein A2 and a minor protein A1. The penetration of the virus into a host cell is quickly followed by translation to produce RdRps and other viral proteins that are required for the production of more viral RNAs. QB ssRNA adsorb to bacterial sex pili proteins and infect. Like other RNA viruses, QB replicates its genome by utilizing virally encoded RNA polymerase (RdRp). The genome is used as the template for the synthesis of other RNA strands. Upon infection, the B-subunit interacts with host proteins to form a complex. The complex contains RNA-helicases to unwind DNA and NTPases that are useful for polymerization. Once the complex forms, the transcription of the genome, a copy of the genome, and mRNAs begin. Phage MS2 has the same genome as QB.

Bacteriophage Qβ coat protein self-assembles to form an icosahedral capsid with a T=3 symmetry, about 26 nm in diameter, and consisting of 89 capsid proteins dimers (178 capsid proteins). It is also involved in viral genome encapsidation through the interaction between a capsid protein dimer and the multiple packaging signals present in the RNA genome. Binding of the capsid proteins to the viral RNA induces a conformational change required for efficient T=3 shell formation. Additionally, it acts as a translational repressor of viral replicase synthesis late in infection. This latter function is the result of capsid protein interaction with an RNA hairpin which contains the replicase ribosome-binding site. See, for example, Gorzelnik et al., Proc Natl Acad Sci USA. 2016 Oct. 11; 113(41):11519-11524; Basnak et al., J Mol Biol. 2010 Feb. 5; 395(5):924-36; and Lim et al., J Biol Chem. 1996 Dec. 13; 271(50):31839-45.

In some embodiments, a VLP derived from bacteriophage Qβ comprise, or consists essentially of, or yet further consists of, a plurality of coat proteins. In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615: MAKLETVTLGNIGKDGKQTLVLNPRGVNPTNGVASLSQAGAVPALEKRVTVSVSQP SRNRKNYKVQVKIQNPTACTANGSCDPSVTRQAYADVTFSFTQYSTDEERAFVRTEL AALLASPLLIDAIDQLNPAY (SEQ ID NO: 28) or an equivalent thereof.

A bacteriophage Qβ hairpin loop refers to a portion of a Qβ RNA where a Qβ coat protein can bind to. In some embodiments, the hairpin loop serves as a packaging signal directing an RNA comprising the hairpin loop to be encapsidated in a capsid comprising, or consisting essentially of, or yet further consisting of a Qβ coat protein.

Accordingly, in some embodiments, the term “with affinity” refers to a nucleic acid (such as a hairpin loop) capable of binding to a bacteriophage Qβ coat protein and serving as a packaging signal directing an RNA comprising the nucleic acid to be encapsidated in a capsid comprising, or consisting essentially of, or yet further consisting of the Qβ coat protein.

A suitable hairpin loop can be determined by ribonuclease T1 degradation of complexes of coat protein and ³²P-RNA obtained by codialysis of the components from urea into buffer solutions. See, for example, Weber H. Biochim Biophys Acta. 1976 Jan. 19; 418(2):175-83. In some embodiments, a Qβ hairpin loop comprises, or alternatively consists essentially of, or yet further consists of AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1).

Cowpea chlorotic mottle virus (CCMV) is a spherical plant virus that belongs to the Bromovirus genus. Several strains have been identified and include, but not limited to, Car1 (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean (S) (Kuhn, 1968. Phytopathology 58:1441-1442), mild (M) (Kuhn, 1979. Phytopathology 69:621-624), Arkansas (A) (Fulton, et al., 1975. Phytopathology 65: 741-742), bean yellow stipple (BYS) (Fulton, et al., 1975. Phytopathology 65: 741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72(9): 768-770). Also, see, for example, WO2021/108202.

In some embodiments, a VLP derived from CCMV comprise, or consists essentially of, or yet further consists of, a plurality of coat proteins. In some embodiments, the coat protein is a wild-type CCMV coat protein, optionally expressed by Car1, Car2, type T, soybean (S), mild (M), Arkansas (A), bean yellow stipple (BYS), R, or PSM strain. In further embodiments, the coat protein is modified, e.g., comprising, or consisting essentially of, or yet further consisting of, one or more substitutions, insertions, and/or deletions. In some cases, the CCMV coat protein comprise, or consists essentially of, or yet further consists of, the sequence as set forth in the UniProtKB ID P03601: MSTVGTGKLTRAQRRAAARKNKRNTRVVQPVIVEPIASGQGKAIKAWTGYSVSKW TASCAAAEAKVTSAITISLPNELSSERNKQLKVGRVLLWLGLLPSVSGTVKSCVTETQ TTAAASFQVALAVADNSKDVVAAMYPEAFKGITLEQLTADLTIYLYSSAALTEGDVI VHLEVEHVRPTFDDSFTPVY (SEQ ID NO: 30), or an equivalent thereof.

In some cases, the engineered VLP from CCMV is prepared by the method as described in Ali et al., “Rapid and efficient purification of Cowpea chlorotic mottle virus by sucrose cushion ultracentrifugation,” Journal of Virological Methods 141: 84-86 (2007).

As used herein, the term “sample” and “biological sample” and “agricultural sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Agricultural samples include soil, foliage or any plant tissue or surface or other sample suspected of harboring virus. In addition, the sample can include industrial samples, such as those isolated from surfaces and the environment.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

In some embodiments, the cell as disclosed herein is a eukaryotic cell or a prokaryotic cell.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The host cell can be a prokaryotic or a eukaryotic cell.

“Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

In one embodiment, the term “disease” or “disorder” as used herein refers to a coronavirus infection, a status of being diagnosed with such infection, a status of being suspect of having such infection, a status of having being exposed to a coronavirus, or a status of at high risk of being exposed to a coronavirus. In one embodiment, the coronavirus is a respiratory virus. In some embodiments, the disease is a SARS-CoV-2 infection or a SARS-CoV-1 infection. In a further embodiment, the disease is Coronavirus disease 2019 (COVID-19) caused by SARS-CoV-2. In yet a further embodiment, the disease is Severe acute respiratory syndrome (SARS) caused by SARS-CoV-1.

In one embodiment, the term “disease” or “disorder” as used herein refers to a symptomatic SARS-CoV-2 infection, a status of being diagnosed with a symptomatic SARS-CoV-2 infection, a status of being suspect of having a symptomatic SARS-CoV-2 infection, or a status of at high risk of having a symptomatic SARS-CoV-2 infection.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, CASCADE BLUE™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

pCDFDuet is a plasmid suitable for bacterial expression and comprising a T7 promoter, and a streptomycin resistance gene. pCDFDuet is designed for the co-expression of two target genes. It is available from EMD Biosciences or Novagen (EMD Millipore). More information of pCDFDuet, including the sequences, maps, and features, is available at www.addgene.org/browse/sequence_vdb/2080/ and www.addgene.org/browse/sequence_vdb/5878/, both of which are last accessed Aug. 20, 2021 and incorporated herein in their entireties.

pET-28a(+) is a plasmid suitable for bacterial expression, comprising a His tag, a Kanamycin resistance gene and an Nterm thrombin cleavage site, and available from EMD Biosciences with catalog number 69864-3. More information of pET-28a(+), including the sequences, maps, and features, is available at www.addgene.org/browse/sequence_vdb/2565/last accessed Aug. 20, 2021 and incorporated herein in its entirety.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., PCT International Application Publication No. WO 95/27071. Ads do not require integration into the host cell genome. Recombinant Ad derived vectors, particularly those that reduce the potential for recombination and generation of wild-type virus, have also been constructed. See, PCT International Application Publication Nos. WO 95/00655 and WO 95/11984, Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters. “Operatively linked” intends the polynucleotides are arranged in a manner that allows them to function in a cell.

A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

Coronaviridae express canonical polymerase genes, as well as structural genes, including S, E, M, and N, but also express a highly divergent set of accessory genes whose open reading frames are interspersed among the structural genes within the 3′ one-third of the viral genome. The accessory genes are thought to contain “luxury” functions that are often not required for in-vitro virus replication. The severe acute respiratory syndrome coronavirus (SARS-CoV) expresses eight such accessory genes (ORF3a, -3b, -6, -7a, -7b, -8a, -8b, and -9b), the most of any known coronavirus. Of these, 7a, 3a and 8 represent the 3^{rd}, 4^{th} and 5^{th} most abundant transcripts behind N and S transcripts.

In some embodiments, the coronavirus as used herein refers to a severe acute respiratory syndrome (SARS) associated coronavirus (SARS-CoV). In some embodiments, the coronavirus is either or both of SARS-CoV-1 and SARS-CoV-2. In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus Ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus.

Severe acute respiratory syndrome coronavirus 2 (SARS-CoV-2), also referred to as 2019 novel coronavirus (2019-nCoV) or human coronavirus 2019 (HCoV-19 or hCoV-19), is the virus that causes COVID-19 (coronavirus disease 2019), the respiratory illness responsible for the COVID-19 pandemic.

Each SARS-CoV-2 virion is 50-200 nanometers in diameter, comprising a linear, positive-sense, single-stranded RNA genome (about 30,000 bases long) and four structural proteins, known as the S (spike), E (envelope), M (membrane), and N (nucleocapsid) proteins. The N protein holds the RNA genome, and the S, E, and M proteins together create the viral envelope. Coronavirus S proteins are glycoproteins that are divided into two functional parts (S1 and S2). In SARS-CoV-2, the spike protein is the protein responsible for allowing the virus to attach to and fuse with the membrane of a host cell; specifically, its S1 subunit catalyzes attachment, the S2 subunit fusion.

Genetic variants of SARS-CoV-2 have been emerging and circulating around the world throughout the COVID-19 pandemic. The B.1.1.7 (Alpha), B.1.351 (Beta), B.1.617.2 (Delta), and P.1 (Gamma) variants circulating in the United States are classified as variants of concern. Other variants are also present, such as B.1.526 (Iota), B.1.427 (Epsilon), B.1.429 (Epsilon), B.1.617 (Kappa, Delta), B.1.525 (Eta), and P.2 (Zeta). Accordingly, the term “SARS-CoV-2” as used herein can refer to any one or more or all of the variants.

A nucleoprotein (i.e., N protein) of a virus, such as SARS-CoV-2, refers to a protein present in the virus and binding to a viral polynucleotide to form a core complex inside the viral particle. The SARS-CoV-2 N protein packages the positive strand viral genome RNA into a helical ribonucleocapsid (RNP) and plays a fundamental role during virion assembly through its interactions with the viral genome and membrane protein M. It also plays an important role in enhancing the efficiency of subgenomic viral RNA transcription as well as viral replication and modulates transforming growth factor-beta signaling by binding host smad3. In some embodiments, a SARS-CoV-2 N protein comprises, or alternatively consists essentially of, or yet further consists of the sequence as set forth in the UniProtKB ID P0DTC9: MSDNGPQNQRNAPRITFGGPSDSTGSNQNGERSGARSKQRRPQGLPNNTASWFTAL TQHGKEDLKFPRGQGVPINTNSSPDDQIGYYRRATRRIRGGDGKMKDLSPRWYFYY LGTGPEAGLPYGANKDGIIWVATEGALNTPKDHIGTRNPANNAAIVLQLPQGTTLPK GFYAEGSRGGSQASSRSSSRSRNSSRNSTPGSSRGTSPARMAGNGGDAALALLLLDR LNQLESKMSGKGQQQQGQTVTKKSAAEASKKPRQKRTATKAYNVTQAFGRRGPEQ TQGNFGDQELIRQGTDYKHWPQIAQFAPSASAFFGMSRIGMEVTPSGTWLTYTGAIK LDDKDPNFKDQVILLNKHIDAYKTFPPTEPKKDKKKKADETQALPQRQKKQQTVTL LPAADLDDFSKQLQQSMSSADSTQA (SEQ ID NO: 31) or an equivalent thereof.

In some embodiments, N1 and N2 refers to a fragment of an N protein. The gene region encoding N1 or N2 were designed to specifically detect SARS-CoV-2 by Centers for Disease Control and Prevention. Accordingly, N1 and N2 are also used herein to refer to the corresponding gene regions encoding N1 and N2, respectively. In some embodiments, the gene region encoding N1 comprises, or consists essentially of, or yet further consists of nucleotide (nt) 28303 to nt 28374 of the genome as set forth in GenBank: MN908947. In some embodiments, the gene region encoding N1 comprises, or consists essentially of, or yet further consists of nt 28287 to 28358 of the genome as set forth in GenBank: MN908947. In some embodiments, the gene region encoding N1 comprises, or consists essentially of, or yet further consists of nt 28271 to nt 28443 of the genome as set forth in NCBI Reference Sequence NC_045512.2. In some embodiments, the gene region encoding N2 comprises, or consists essentially of, or yet further consists of nt 29180 to nt 29246 of the genome as set forth in GenBank: MN908947. In some embodiments, the gene region encoding N2 comprises, or consists essentially of, or yet further consists of nt 29164 to nt 29230 of the genome as set forth in GenBank: MN908947. In some embodiments, the gene region encoding N1 comprises, or consists essentially of, or yet further consists of nt 29091 to nt 29230 of the genome as set forth in NCBI Reference Sequence NC_045512.2. See, for example, Emerging Infectious Diseases. 2020; 26(8): 1654-1665. In further embodiments, the gene region encoding N1 comprises, or consists essentially of, or yet further consists of AAAATGTCTGATAATGGACCCCAAAATCAGCGAAATGCACCCCGCATTACGTTT GGTGGACCCTCAGATTCAACTGGCAGTAACCAGAATGGAGAACGCAGTGGGGCG CGATCAAAACAACGTCGGCCCCAAGGTTTACCCAATAATACTGCGTCTTGGTTCA CCGCTCTCAC (nt 47 to nt 219 of SEQ ID NO: 1). In some embodiments, the gene region encoding N2 comprises, or consists essentially of, or yet further consists of any one of:

CTTTCGGCAG ACGTGGTCCA GAACAAACCC AAGGAAATTT
TGGGGACCAG GAACTAATCA GACAAGGAAC TGATTACAAA
CATTGGCCGC AAATTGCACA ATTTGCCCCC AGCGCTTCAG
CGTTCTTCGG AATGTCGCGC (nt 220 to nt 359 of
SEQ ID NO: 1),
or
GTAACACAAG CTTTCGGCAG ACGTGGTCCA GAACAAACCC
AAGGAAATTT TGGGGACCAG GAACTAATCA GACAAGGAAC
TGATTACAAA CATTGGCCGC AAATTGCACA ATTTGCCCCC
AGCGCTTCAG CGTTCTTCGG AATGTCGCGC ATTGGCATGG
AAGTCACACC TTCGGGAACG TGGTTGACCT ACACAGGTGC
CATCAAATTG GATGACAAAG ATCCAAATTT CAAAGATCAA
(nt 226 to nt 465 of SEQ ID NO: 2).

Human ribonuclease P protein subunit p30 (also referred to herein as human ribonuclease P, human RNase P, or RP, or RPP30) is a component of ribonuclease P, a ribonucleoprotein complex that generates mature tRNA molecules by cleaving their 5′-ends. It is also a component of the MIRP ribonuclease complex, which cleaves pre-rRNA sequences. Non-limiting exemplary sequences of this protein or the underlying gene can be found under Gene Cards ID: GC10P090871, HGNC: 17688, NCBI Entrez Gene: 10556, Ensembl: ENSG00000148688, OMIM®: 606115, or UniProtKB/Swiss-Prot: P78346, each of which is incorporated by reference herein in its entirety. Detecting presence of and/or quantifying the RP gene or a fragment (also referred to herein as a region) can serve as a positive control when detecting presence of and/or quantifying SARS-CoV in a biological sample. For example, a positive detection of the RP gene in the biological sample indicates one or more of the following: a successful nucleic acid extraction, a specimen quality suitable for detection, or lack of reaction inhibitors in the biological sample. In some embodiments, the RP gene or a fragment thereof comprises, or consists essentially of, or yet further consists of nt 1 to nt 280 of the genome as set forth in NCBI Reference Sequence NM_006413, see for example, nt 360 to nt 639 of SEQ ID NO: 1. In some embodiments, the RP gene or a fragment thereof comprises, or consists essentially of, or yet further consists of any one of:

ATGGGACTTC AGCATGGCGG TGTTTGCAGA TTTGGACCTG
CGAGCGGGTT CTGACCTGAA GGCTCTGCGC GGACTTGTGG
AGACAGCCGC TCACCTTGGC TATTCAGTTG TTGCTATCAA
TCATATCGTT GACTTTAAGG AAAAGAAACA GGAAATTGAA
AAACCAGTAG CTGTTTCTGA ACTCTTCACA ACTTTGCCAA
TTGTACAGGG AAAATCAAGA CCAATTAAAA TTTTAACTAG
ATTAACAATT ATTGTCTCGG ATCCATCTCA CTGCAATGTT
(nt 360 to nt 639 of SEQ ID NO: 1),
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTGGGGG
TAATTGTCTC TTGGTGGGCC CAGTTAGTGG GCCTTCCTGA
GTGTGTGTAT GCGGTCTGTA ACTATTGCCA TATAAATAAA
AAATCCTGTT GCACTAGTGT CCTGCCATCC (nt
472 to nt 821 of SEQ ID NO: 2),
or
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTG
(nt 472 to nt 707 of SEQ ID NO: 2).

Symptoms of a coronavirus infection include, but are not limited to, mild symptoms, such as fatigues, tingling, tingling or numbness in the hands and feet, dizziness, confusion, brain fog, body ache, chills, loss of appetite, nausea, vomiting, abdominal pain or discomfort, loss of smell, inability to taste, muscle weakness, photophobia, adenopathy, headaches, cough, dry cough, shortness of breath, sore throat, lower extremity weakness/numbness, diarrhea, low blood O₂, sneezing, runny nose or post-nasal drip; severe symptoms, such as ventilatory use, high fever, severe cough, delirium, seizures, stroke, systematic inflammation, cytokine storm; and other symptoms, such as fever, swollen adenoids, pneumonia, bronchitis, and Dyspnea.

Viral infection of a coronavirus, such as SARS-COV-2, can be detected via a commercially available test known in the art, for example via polymerase chain reaction (PCR) or immunoassay may be used. In some embodiments, a method as disclosed herein further comprises detecting a coronavirus via a test known in the art. In one embodiment, active viral infection refers to an ongoing infection wherein the virus is making copies of itself and producing new virus. Such active viral infection may be detected via polymerase chain reaction (PCR). Non-limiting examples of primers and probes suitable for use in the PCR include 2019-nCoV CDC Probe and Primer Kit for SARS-CoV-2 (BioSearch Technologies, Catalog No. KIT-nCoV-PP1-1000), 2019-nCoV Kit, 500 rxn (Integrated DNA Technologies (IDT), Catalog No. 10006606) and 2019-nCoV Kit, 1000 rxn (Integrated DNA Technologies (IDT), Catalog No. 10006770). Also see, www.cdc.gov/coronavirus/2019-ncov/lab/rt-pcr-panel-primer-probes.html and www.cdc.gov/coronavirus/2019-ncov/downloads/List-of-Acceptable-Commercial-Primers-Probes.pdf. Suitable protocols for performing such tests can be found at www.cdc.gov/coronavirus/2019-ncov/lab/virus-requests.html, www.fda.gov/media/134922/download, www.cdc.gov/coronavirus/2019-ncov/downloads/processing-sputum-specimens.pdf, www.fda.gov/media/134922/download, and www.fda.gov/media/134919/download, www.fda.gov/media/134922/download, last accessed on Aug. 10, 2021, or Update: FDA Authorizes First Diagnostic Test Using At-Home Collection of Saliva Specimens, available at www.fda.gov/news-events/press-announcements/coronavirus-covid-19-update-fda-authorizes-first-diagnostic-test-using-home-collection-saliva, last accessed Jul. 16, 2020. In some embodiments, diagnostic assays for COVID-19 based on detecting antibodies may be combined with the disclosure herein, such as those discussed by Lisboa Bastos M et al. (Diagnostic accuracy of serological tests for covid-19: systematic review and meta-analysis. BMJ. 2020 Jul. 1; 370:m2516. doi: 10.1136/bmj.m2516). 10.1.571 Other commercially available tests include, but not limited to those listed in the Table below.

TABLE 1
Commercially available tests for SARS-COV-2 and COVID-19.

| Company Name | Test Name |
|---|---|
| 3D Medicines | SARS-COV-2 and Influenza A & B RT-qPCR |
| | Detection Kit |
| Abbott | SARS-COV-2 IgG test |
| Abbott | ID Now COVID-19 |
| Abbott | Abbott RealTime SARS-COV-2 EUA test |
| Anatolia Geneworks | Bosphore Novel Coronavirus (2019-nCoV) Detection |
| | Kit |
| ARUP Laboratories | COVID-19 |
| A*STAR, Tan Tock Seng | A*STAR Fortitude 2.0 |
| Hospital of Singapore | |
| Assure Tech | COVID-19 IgG/IgM Rapid Test Device |
| Atila BioSystems | iAMP COVID-19 Detection Kit |
| AusDiagnostics | AusDiagnostics SARS-COV-2, influenza, RSV panel |
| Autobio Diagnostics | Anti-SARS-COV-2 Rapid Test |
| Avellino Lab | Avellino SARS-COV-2/COVID-19 (AvellinoCoV2) |
| Bako Diagnostics | BakoDx SARS-COV-2 RNA test |
| Baptist Hospital Miami | COVID-19 RT-PCR Test |
| Pathology/Laboratory Medicine | |
| Lab | |
| Becton Dickinson | BD SARS-COV-2 Reagents for BD MAX System |
| Becton Dickinson, BioGx | BioGX SARS-COV-2 Reagents for the BD MAX |
| | System |
| Beijing Decombio Biotechnology | Novel Coronavirus IgM/IgG Combo Rapid Test- |
| | Cassette |
| Beijing Diagreat Biotechnologies | 2019-nCoV IgG, IgM Antibody Determination Kits |
| | 2019-nCoV IgG/IgM Antibody Rapid Test Kit |
| Beijing Kewei Clinical | Genonto RapidTest10 COVID-19 IgG/IgM Antibody |
| Diagnostic Reagent | Rapid Test Kit |
| Beijing O&D Biotech | Coronavirus disease (COVID-19) Total Antibody |
| | Rapid Test (Colloidal Gold) |
| Beroni Group | SARS-COV-2 IgG/IgM Antibody Detection Kit |
| BGI | Real-Time Fluorescent RT-PCR kit for detecting |
| | SARS-2019-nCoV |
| Biodesix | SARS-COV-2 Droplet Digital PCR (ddPCR) test |
| Biolidics | 2019-nCoV IgG/IgM Detection Kit (Colloidal Gold) |
| BioMédomics | COVID-19 IgM-IgG Rapid Test |
| BioMérieux | SARS-COV-2 R-GENE test |
| BioMerieux/BioFire Defense | BioFire COVID-19 test |
| Bioneer | AccuPower COVID-19 Real-Time RT-PCR Kit, |
| | AccuPower SARS-COV-2 Real-Time RT-PCR Kit |
| Bio-Rad Laboratories | SARS-COV-2 Total Ab test |
| BioReference Laboratories | Novel Coronavirus COVID-19 |
| Boston Children's Hospital | Childrens-Altona-SARS-CoV-2 assay |
| Infectious Diseases Diagnostic | |
| Laboratory (IDDL) | |
| BTNX | Rapid Response COVID-19 IgG/IgM Test Cassette |
| Cellex | qSARS-COV-2 IgG/IgM Rapid Test |
| Centers for Disease Control and | CDC 2019-Novel Coronavirus (2019-nCoV) Real- |
| Prevention (performed at | Time RT-PCR Diagnostic Panel (CDC) |
| qualified high-complexity CLIA | |
| laboratories designated by CDC) | |
| Cepheid | Xpert Xpress SARS-COV-2 test |
| CerTest BioTec | ViaSure SARS-COV-2 Real Time PCR Detection Kit |
| Chembio Diagnostics | DDP COVID-19 IgM/IgG System |
| Children's Hospital of | SARS-COV-2 RT-PCR test |
| Philadelphia Infectious Disease | |
| Diagnostics Laboratory | |
| ChromaCode | HDPCR SARS-COV-2 real-time PCR assay |
| CirrusDx Laboratories | CirrusDx SARS-COV-2 Assay |
| Co-Diagnostics | Logix Smart Coronavirus Disease 2019 (COVID-19) |
| | Kit |
| Core Technology | CoreTest COVID-19 IgM/IgG Ab Test |
| Credo Diagnostics Biomedical | VitaPCR SARS-COV2 Assay |
| DiaCarta | Quanti Virus SARS-COV-2 test kit |
| Diagnostic Solutions Laboratory | COVID-19 Assay |
| DiaSorin Molecular | Simplexa COVID-19 Direct |
| Diatherix Eurofins | COVID-19 Panel |
| Diazyme Laboratories | Diazyme DZ-LITE SARS-COV-2 IgG, IgM CLIA |
| | Kits |
| Eachy Biopharmaceuticals | AccuRapid SARS-COV-2 IgM/IgG Test Kit (Lateral |
| | Flow Immunoassay) |
| Euroimmun/PerkinElmer | EuroRealTime SARS-COV-2 |
| Euroimmun/PerkinElmer | Anti-SARS-COV-2 ELISAs (IgA and IgG) |
| Exact Sciences | SARS-COV-2 Test |
| Fosun Pharma USA | Fosun COVID-19 RT-PCR Detection Kit |
| Fulgent Genetics/MedScan | COVID-19 |
| Laboratory | |
| Genetic Signatures | EasyScreen SARS-COV-2 detection kit |
| Genetron | Detection Kit for Novel Coronavirus (SARS-COV-2) |
| | RNA (PCR-Fluorescence Probing) |
| GenMark Diagnostics | ePlex SARS-COV-2 Test |
| Genomica/PharmMar Group | 2 kits: qCOVID-19, CLART COVID-19 |
| GenoSensor | GS COVID-19 RT-PCR Kit |
| Gnomegen | Gnomegen COVID-19 RT-Digital PCR Detection Kit |
| Gold Standard Diagnostics | SARS-COV-2 IgG, IgM, IgA assays |
| Guangzhou Wondfo Biotech | SARS-COV-2 Antibody Test |
| Hackensack University Medical | CDI Enhanced COVID-19 Test |
| Center (HUMC) Molecular | |
| Pathology Laboratory | |
| Hangzhou AllTest Biotech | AllTest 2019-nCoV IgG/IgM Rapid Test Cassette, |
| | AllTest COVID IgG/IgM Rapid Test Dipstick |
| Hangzhou Biotest Biotech | COVID-19 IgG/IgM Rapid Test Cassette |
| Hangzhou Clongene Biotech | Clungene COVID-19 IgM/IgG Rapid Test Cassette |
| Hangzhou Testsealabs | One Step SARS-COV2 (COVID-19) IgG/IgM Test |
| Biotechnology | |
| Healgen Scientific | COVID-19 IgG/IgM Rapid Test Cassette(Whole |
| | Blood/Serum/Plasma) |
| Hologic | Panther Fusion SARS-COV-2 assay |
| InBios International | Smart Detect SARS-COV-2 rRT-PCR Kit |
| Innovita (Tangshan) Biological | 2019-nCoV Ab Test (Colloidal Gold) |
| Technology | |
| Integrated DNA | IDT 2019-novel coronavirus kit |
| Technologies/Danaher | |
| Integrity Laboratories | SARS-COV-2 Assay |
| Ipsum Diagnostics | COV-19 IDx assay |
| Jiangsu Macro & Micro-Test | SARS-COV-2 IgM/IgG Rapid Assay Kit (Colloidal |
| Med-Tech | Gold) |
| JN Medsys | ProTect Covid-19 kit |
| Kogene Biotech | 2019 Novel Coronavirus Real-time PCR Kit |
| KorvaLabs | Curative-Korva SARS-Cov-2 Assay |
| Laboratory Corporation of | LabCorp 2019 Novel Coronavirus test |
| America | |
| LGC, Biosearch Technologies | 2019-nCoV CDC-qualified Probe and Primer Kits for |
| | SARS-COV-2 |
| Lifeassay Diagnostics | Test-it COVID-19 IgM/IgG Lateral Flow Assay |
| Luminex | ARIES SARS-COV-2 Assay |
| Luminex | NxTAGCoV Extended Panel Assay |
| Maccura Biotechnology | SARS-COV-2 Fluorescent PCR Kit |
| Massachusetts General Hospital | MGH COVID-19 qPCR assay |
| Medical Systems Biotechnology | Coronavirus Disease 2019 Antibody (IgM/IgG) |
| | Combined Test Kit |
| Mesa Biotech | Accula SARS-COV-2 test |
| Mount Sinai Labs | COVID-19 ELISA IgG Antibody Test |
| Nanjing Liming Bio-products | SARS-COV-2 IgM/IgG Antibody Rapid Test Kit |
| NanoResearch | NanoMedicina SARS-COV-2 IgM/IgG Antibody |
| | Rapid Test |
| Nantong Diagnos Biotechnology | (2019-nCoV) New coronavirus Antibody Test |
| | (Colloidal Gold) |
| NeuMoDx Molecular | NeuMoDx SARS-CoV-2 Assay |
| Nirmidas Biotech | COVID-19 (SARS-COV-2) IgM/IgG Antibody |
| | Detection Kit |
| Northwestern Medicine | SARS-Cov-2 Assay |
| Diagnostic Molecular Laboratory | |
| Novacyt/Primerdesign | COVID-19 Genesig Real-Time PCR assay |
| NY State Department of Health | New York SARS-COV-2 Real-time Reverse |
| (performed at Wadsworth Center | Transcriptase (RT)-PCR Diagnostic Panel |
| and New York City Department | |
| of Health and Mental Hygiene, | |
| Public Health Laboratories) | |
| Orig3n | Orig3n 2019 Novel Coronavirus (COVID-19) Test |
| Ortho Clinical Diagnostics | Vitros Immunodiagnostic Products Anti-SARS-CoV-2 |
| | Total Reagent Pack and Calibrators |
| Osang Healthcare | GeneFinder COVID-19 Plus RealAmp Kit |
| PathoFinder | RealAccurate Quadruplex Corona-plus PCR Kit |
| PCL | COVID19 IgG/IgM Rapid Gold |
| PerkinElmer | PerkinElmer New Coronavirus Nucleic Acid |
| | Detection Kit |
| Phamatech | COVID19 IgG/IgM Rapid Test |
| Promedical | COVID-19 Rapid Test, Wondfo SARS-COV-2 |
| | Antibody Test (Lateral Flow Method) |
| Qiagen | QiaStat-Dx Respiratory SARS-COV-2 Panel |
| Quest Diagnostics | Coronavirus Disease 2019 (COVID-19) Test |
| Quidel | Lyra SARS-COV-2 Assay |
| Rendu Biotechnology | 2019-nCoV detection kit |
| Roche | Cobas SARS-COV-2 Test |
| Rutgers University Clinical | ThermoFisher - Applied Biosystems TaqPath COVID- |
| Genomics Laboratory | 19 Combo Kit |
| ScienCell Research Laboratories | ScienCell SARS-COV-2 Coronavirus Real-time RT- |
| | PCR (RT-qPCR) Detection Kit |
| SD Biosensor | Standard Q COVID-19 IgM/IgG Duo |
| Seegene | Allplex 2019-nCoV Assay |
| Sentinel Diagnostics | STAT-NAT COVID-19 HK kit, B kit |
| Shanghai Fosun Long March | novel coronavirus nucleic acid detection kit |
| Medical Science/Shanghai Fosun | |
| Pharmaceutical | |
| Shenzhen Landwind Medical | COVID-19 IgG/IgM Rapid Test Device |
| Snibe Diagnostics | Maglumi 2019-nCoV (SARS-COV-2) IgM/IgG kits |
| SolGent | DiaPlexQ Novel Coronavirus (2019-nCoV) Detection |
| | kit |
| Specialty Diagnostic (SDI) | SDI SARS-COV-2 Assay |
| Laboratories | |
| Stanford Health Care Clinical | SARS-COV-2 PCR Assay |
| Virology Laboratory | |
| SureScreen Diagnostics | SureScreen COVID19 IgM/IgG Rapid Test Cassette |
| Suzhou Kangheshun Medical | SARS-COV-2 IgG/IgM Rapid Test Cassette |
| Technology | |
| Systaaq Diagnostic Products | 2019-Novel Coronavirus (COVID-19) Real Time PCR |
| | Kit |
| Telepoint Medical Services | SARS-COV-2 IgG/IgM Rapid Qualitative Test |
| Thermo Fisher Scientific | TaqPath COVID-19 Combo Kit, RT-PCR CE-IVD Kit |
| Tianjin Beroni Biotechnology | SARS-COV-2 IgG/IgM Antibody Detection Kit |
| TIB Molbiol Syntheselabor | Sarbecovirus E-gene |
| Trax Management Services | Phoenix Dx 2019-CoV |
| United Biomedical | UBI SARS-COV-2 ELISA |
| University of North Carolina | UNC Health SARS-COV-2 real-time RT-PCR test |
| Medical Center | |
| Vela Diagnostics | ViroKey SARS-COV-2 RT-PCR Test |
| Viracor Eurofins | Viracor SARS-COV-2 assay |
| Vision Medicals | SARS-COV-2 Clinical Sequencing assay |
| VivaChek Biotech (Hangzhou) | VivaDiag COVID-19 IgM/IgG Rapid Test |
| Yale New Haven Hospital | SARS-COV-2 PCR test |
| Clinical Virology Laboratory | |
| YD Diagnostics | MolecuTech Real-Time COVID-19 |
| Zhejiang Orient Gene Biotech | COVID-19 IgG/IgM Rapid Test Cassette |
| Zhengzhou Fortune Bioscience | IgG/IgM Antibody Rapid Test Kits (Colloidal Gold |
| | Immunochromatography method) |
| Zhongshan Bio-Tech | SARS-COV-2 IgM/IgG (GICA) |
| Zhuhai Encode Medical | Novel Coronavirus (COVID-19) IgG/IgM Rapid Test |
| Engineering | Device |
| Zhuhai Livzon Diagnostics | Diagnostic Kit for IgM/IgG Antibody to Coronavirus |
| | (SARS-COV-2) (Colloidal Gold) |

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

A therapy for suppressing an immune response of the subject, suppressing viral replication, ameliorating coronavirus-associated symptoms, or treating the coronavirus are available in the art. An immunotherapy in treating a coronavirus and/or NK cell based immunotherapy therapies in treating a coronavirus may be included, such as those shown in clinical trials with an identifier of NCT04324996, NCT04344548, NCT04375176, NCT04280224, NCT04365101 or NCT04470999. More examples are provided in the Table below.

As used herein, a reading frame refers to a contiguous and non-overlapping set of three-nucleotide codons in a polynucleotide. Because a three-nucleotide codon encodes one amino acid, there exist three possible reading frames for a given coding polynucleotide, i.e., reading frame 1 starting from the first nucleotide (i.e., the first three nucleotides constitutes the first codon), reading frame 2 starting from the second nucleotide (i.e., the second to the fourth nucleotides constitutes the first codon), and reading frame 3 starting from the third nucleotide (i.e., the third to the fifth nucleotides constitutes the first codon). According, different peptides are encoded if the reading frame is different. Therefore, a polynucleotide encoding a protein or a fragment thereof is considered as in frame if such polynucleotide encodes the protein or fragment thereof, for example when the polynucleotide is operatively linked to a regulatory sequence and/or in a plasmid. Otherwise, a polynucleotide encoding a protein or a fragment thereof is considered as out-of-frame if such polynucleotide does not encodes the intended protein or fragment thereof, for example when the polynucleotide is operatively linked to a regulatory sequence and/or in a plasmid.

In some embodiments, a “ribosome binding site” refers to a sequence of nucleotides upstream of the start codon of an RNA transcript that is responsible for the recruitment of a ribosome during the initiation of protein translation. In some embodiments, a ribosome binding site as used herein refers to a nucleic acid region operatively linked to an RNA-coding nucleic acid region, when transcribed, allows recruitment of a ribosome to the encoded RNA.

“Upstream” and “5′ to” are used interchangeably and refer to the relative direction toward the 5′ end of a nucleic acid, while “downstream” and “3′ to” are used interchangeably and refer to the opposite relative direction. In some embodiments, a first nucleic acid region that is upstream or downstream to a second nucleic acid region can be immediately adjacent to the second nucleic acid region. Alternatively, the first nucleic acid region is not immediately adjacent to the second nucleic acid region. I.e., the two regions can be separated by one or more nucleic acid residue.

### Modes for Carrying Out the Disclosure

Coronavirus disease 2019 (COVID-19) is a highly transmissible disease that has affected more than 90% of the countries worldwide. At least 17 million individuals have been infected, and some countries are still battling first or second waves of the pandemic. Nucleic acid tests, especially reverse-transcription polymerase chain reaction (RT-PCR), have become the workhorse for early detection of COVID-19 infection. Several RT-qPCR primer/probe sets that target distinct regions of the viral genome have been developed worldwide for SARS-CoV-2 detection. Testing for the nucleocapsid phosphoprotein (N) yields the most consistent test results, as it is highly conserved (Comparison of National RT—PCR Primers, Probes, and Protocols for SARS-CoV-2 Diagnostics; Johns Hopkins Center for Health Security, available at www.centerforhealthsecurity.org/resources/COVID-19/COVID-19-fact-sheets/200410-RT-PCR.pdf, last accessed on Aug. 17, 2021). The comparative sensitivity and efficiency of the different primer/probe sets were recently reviewed. See, e.g., Vogels et al., Nat Microbiol. 2020 October; 5(10):1299-1305. The primer/probe sets developed by the Centers for Disease Control and Prevention (CDC) target two regions in the N gene: N1 at gene location 28287-28358 nucleotides (nt) and N2 at gene location 29164-29230 nt. The Human RNase P gene at gene location 28-92 nt serves as an endogenous control. Overall, these primer/probe sets are validated and yield consistent results with clinical samples. See, e.g., Vogels et al. However, it remains challenging to reliably identify false negative results. See, for example, Tahamtan, A.; Ardebili, A. Real-Time RT-PCR in COVID-19 Detection: Issues Affecting the Results. Expert Rev. Mol. Diagn. 2020, 20, 453-454.

Validation of diagnostic tests is crucial to ensure test accuracy and precision. Positive controls should be run in parallel with patient samples as a benchmark to monitor and validate test accuracy. The lack of reliable positive controls is a key problem for RT-PCR-based viral testing. See, e.g., Reusken et al., Euro Surveill. 2020 February; 25(6):2000082. While some laboratories use infectious viral RNA extracted from highly positive patient samples, the most commonly used controls are commercially available synthetic RNA transcripts or plasmids. See, e.g., CDC assays for SARS-CoV-2 detection available at www.idtdna.com/pages/landing/coronavirus-research-reagents/cdc-assays, last accessed on Jul. 15, 2020; and Thermo Fisher Scientific Launches New AcroMetrix Coronavirus 2019 (COVID-19) RNA Control (RUO) to Monitor and Validate COVID-19 Molecular Diagnostic Tests available at www.biospace.com/article/releases/thermo-fisher-scientific-launches-new-acrometrix-coronavirus-2019-covid-19-rna-control-ruo-/, last accessed on Aug. 17, 2021. However, these samples have shortcomings. Because RNA is an inherently unstable molecule, both infectious viral RNA and synthetic RNA transcripts are labile and susceptible to degradation. Also, the multiple freeze-thaw cycles to which these samples are subjected can further degrade RNA quality. See, for example, Yu et al., Cell Tissue Bank. 2017 September; 18(3):433-440. Whereas RNA stabilizers such as PAXGENE®, RNAlater (see, e.g., Williams M A. Biomark Insights. 2010 Dec. 1; 5:139-43) and DNA/RNA SHIELD™ (Research, Z. DNA/RNA SHIELD™, available at files.zymoresearch.com/protocols/_r1100-50_r1100-250_r1200-25_r1100-125_dna_rna_shield.pdf, last accessed Jul. 16, 2020) or production of RNA in dry form (Fabre et al., Eur J Hum Genet. 2014 March; 22(3):379-85) have improved the stability of RNA, the requirement for cold-chain distribution remains expensive and challenging, especially in underserved areas or regions with tropical climates. While plasmid DNA is more stable than RNA, neither positive controls enables concurrent processing with patient samples, or full-process control. Because RNA/DNA samples are only added downstream in the testing process, they cannot control for whether the viral RNA was degraded during the initial extraction steps.

To overcome these shortcomings, disclosed herein is a biomimetic nanotechnology solution, i.e. to package RNA transcripts containing the nucleic acid regions for binding of the SARS-CoV-2 primers and probes into a nanoparticle carrier. More specifically, a biomimetic positive control was generated by developing a virus-like particle (VLP) technology that takes advantage of the unique features of SARS-CoV-2 but is non-infectious and safe to use in diagnostic assays. While many nanotechnology platform technologies are available, including polymer and lipid nanoparticles, that could carry nucleic acids, the capsids from viruses naturally evolved to package nucleic acid and thus make a suitable choice. 10.1691 Specifically, biomimicry was achieved by packaging non-infectious, replication-deficient synthetic SARS-CoV-2 RNA target sequences into non-infectious VLPs from the bacteriophage Qbeta (Qβ) and the plant virus cowpea chlorotic mottle virus (CCMV). Both VLPs measure ˜30 nm and have been extensively studied in nanotechnology. See, e.g., Chung et al., Adv Drug Deliv Rev. 2020; 156:214-235. Encapsidation into a viral capsid more closely mimics the conditions encountered by the RNA template of SARS-CoV-2 within clinical or environmental samples. This approach has been reported previously: for example, cowpea mosaic virus (CPMV) capsids were used to encapsidate target RNA for detection of foot-and-mouth disease virus (FMDV, see, e.g., Madi et al., J Virol Methods. 2015 August; 220:27-34), tobacco mosaic virus (TMV) was developed as a positive control for Ebola diagnostic assays (see, e.g., Lam et al., Sci Rep. 2016 Mar. 31; 6:23803), and bacteriophage Qβ was explored as a control for foodborne virus detection (see, e.g., Yao et al., Food Environ Virol. 2019 December; 11(4):383-392). In response to COVID-19, Asuragen and SeraCare have announced developments of SARS-CoV-2 positive controls in which RNA targets are encapsidated into bacteriophage VLPs (see, e.g., Asuragen Develops Armored RNA Quant® SARS-CoV-2 Control, available at www.biospace.com/article/releases/asuragen-develops-armored-rna-quant-sars-cov-2-control/, last accessed on Jul. 15, 2020) or a replication-deficient mammalian virus (see, e.g., AccuPlex™ SARS-CoV-2 Reference Material Kit, available at www.seracare.com/AccuPlex-SARSCoV2-Reference-Material-Kit-0505-0126/, last accessed on Jul. 15, 2020) or Xu et al., Front Bioeng Biotechnol. 2020 Jul. 30; 8:862. doi: 10.3389/fbioe.2020.00862. Erratum in: Front Bioeng Biotechnol. 2020 Sep. 9; 8:1026). Of note is also the development of VLPs for vaccine development (see, e.g., Swann et al., Sci Rep. 2020 Dec. 14; 10(1):21877. Erratum in: Sci Rep. 2021 Apr. 26; 11(1):9352; and Yeh et al., Molecular Therapy. Vol 11, Supplement 1, May 2005); research from academia and industry has led to the development of more than 200 vaccine candidates in record time, many designs rely on nanotechnology concepts and various VLP subunit vaccines are being developed (see., e.g., Shin et al., Nat Nanotechnol. 2020 August; 15(8):646-655; and Qian et al., Vaccines (Basel). 2020 Mar. 20; 8(1):139).

Disclosed herein is a synthetic SARS-CoV-2 detection module (SDM). The positive controls comprise, or alternatively consist essentially of, or yet further consist of synthetic SDM that comprises, or alternatively consists essentially of, or yet further consists of all nucleic acid regions for binding of the CDC-designated RT-qPCR primers and probes or other suitable primers and probes. In one non-limiting example, the SDM module was then was encapsidated into VLPs by in vitro or in vivo reconstitution of chimeric VLPs, yielding three SARS-CoV-2 positive controls termed Qβ 1P-C19, Qβ 2P-C19 (synthesized from a one or two plasmid system, respectively), and CCMV-C19. While in vitro reconstitution of viral capsids around a synthetic RNA template is well established (and was the method used for CCMV), the in vivo reconstitution is less frequently reported. However, the latter requires fewer processing steps and thus may be higher yielding and more economic to mass produce. To achieve efficient in vivo reconstitution, careful design considerations need to be taken in account, such as length of the target RNA and its molecular features, i.e. deletion of ribosome binding sites (for enhanced safety) and appendage of Qβ hairpin (for encapsidation). See, e.g., Fang et al., RSC Advances. 2018, 8, 21399-21406. Developed herein are such methods for the Qβ VLP system. The structural integrity and stability of the resulting nanoparticle positive controls were assessed over time using a combination of transmission electron microscopy (TEM), size exclusion chromatography (SEC), dynamic light scattering (DLS) and electrophoretic mobility assays. A set of RT-PCR assays was performed to validate the biomimetic SARS-CoV-2 positive controls. Lastly, the positive controls were subjected to screening and used as external controls for patient sample testing.

Unlike standard quantitative RT-PCR (RT-qPCR) methods, consensus detection regions for RT-LAMP assays have not been recommended by the Centers for Disease Control and Prevention (CDC). Positive controls are therefore tailored according to the gene regions used in different RT-LAMP assays, making this a commercially non-profitable approach. Internal and external positive controls are used in PCRs to identify false negatives. See, e.g., Lion, Leukemia. 2001 July; 15(7):1033-7. However, internal positive controls are usually omitted from RT-LAMP assays, possibly because a suitable primer/probe set has not been validated or because a suitable housekeeping gene has not been identified. Accordingly, secondary validation methods are needed to confirm RT-LAMP results, such as LAMP sequencing (Dao et al., Sci Transl Med. 2020 Aug. 12; 12(556):eabc7075), RT-qPCR (Kitagawa et al., J Clin Virol. 2020 August; 129:104446), or agarose gel electrophoresis (Baek et al). Without the pairing of RT-qPCR primer/probe sets, validation of the RT-LAMP internal positive control is challenging. The human p-actin gene was found to be unsuitable as an internal control for clinical samples, suggesting other human genes should be tested as internal control candidates for RT-LAMP. See, e.g., Huang et al., Microb Biotechnol. 2020 July; 13(4):950-961.

The commercially approved RT-LAMP test for the detection of SARS-CoV-2 includes synthetic RNAs as external positive controls. See, e.g., Emergency Use Authorization (EUA) Summary for the COLOR SARS-CoV-2 LAMP Diagnostic Assay as cited herein, AQ-TOP™ COVID-19 Rapid Detection Kit as cited herein, and MobileDetect-BIO BCC19 Test Kit—Instructions for Use available at www.fda.gov/media/141791/download, last accessed one Nov. 15, 2020. However, these controls are inherently unstable and unable to serve as full process controls throughout the steps of RNA extraction through to DNA amplification. To overcome this issue of instability, developed herein are virus-like particles (VLPs) based on bacteriophage Qβ and the plant virus cowpea chlorotic mottle virus (CCMV) for the encapsidation of truncated SARS-CoV-2 RNAs to serve as full process controls for RT-qPCR and RT-LAMP. See, e.g., Chan et al., ACS Nano. 2021 Jan. 26; 15(1):1259-1272. Epub 2020 Nov. 25.

VLPs are naturally occurring nucleic acids carriers that protect their RNA cargo from degradation by RNases. See, e.g., Fang et al., RSC Adv. 2018, 8, 21399-21406; and Biddlecome et al., PLoS One. 2019 Jun. 4; 14(6):e0215031. VLPs are non-infectious and can be manufactured at high yields through fermentation in microbial culture or molecular farming in plants. See, e.g., Chung et al., Adv Drug Deliv Rev. 2020; 156:214-235. VLPs serve as a nanotechnology platform with unique features compared to contemporary approaches that use lipids (Barba et al. Pharmaceutics. 2019 Jul. 24; 11(8):360) or polymers (Raval et al., Sci Rep. 2019 Nov. 5; 9(1):16047) for RNA packaging. A key advantage is the natural property of VLPs to package and stabilize RNA, and their high degree of stability avoids the need of cold chain storage or transport. The VLPs mimic the infectious pathogen; the coat proteins protect the RNA cargo, and therefore VLP-based controls can be used as full process control, i.e. the VLPs can be handled exactly like patient samples. See, e.g., Chan et al., ACS Nano. 2021 Jan. 26; 15(1):1259-1272. For these reasons VLPs have been chosen for the development and implementation of RT-LAMP assays with integrated full-process controls.

Exemplified herein is a SARS-CoV-2 LAMP Detection Module (SLDM) in which truncated SARS-CoV-2 nucleocapsid (N) and human RNase P (RP) genes are incorporated into Qβ and CCMV VLPs. See, e.g., Chan et al. These detection regions include binding sites for RT-qPCR and RT-LAMP primers, enabling validation of both techniques in parallel. The RP detection region serves as an internal control for both assays. The encapsidation of the SLDM into VLPs was used to produce three alternative positive control reagents: Qβ 1P-C19L, Qβ 2P-C19L, and CCMV-C19L (the difference between the two Qβ-based constructs is whether these are expressed from a single or two-plasmid system as elaborated herein).

The RT-LAMP products were detected in a colorimetric reaction by observing a pH-dependent color change or by measuring the difference in absorbance at 434 and 560 nm. In the presence of target RNA, phenol red in the RT-LAMP reagent changes in color from pink to yellow due to the accumulation of H⁺ ions during amplification. See, e.g., Tanner et al. Biotechniques. 2015 Feb. 1; 58(2):59-68. Differences between the absorbance at 434 and 560 nm were tabulated as precise values, with a threshold of ≥0.3 for the positive detection of SARS-CoV-2 RNA. See, e.g., Dao et al. It was also confirmed the success of direct RT-LAMP (also known as the “swab-to-test assay”) on COVID-19 clinical samples heated to 95° C. for 5 min to release RNA, avoiding the need for a specific RNA extraction step. The released RNAs could be detected by RT-qPCR, and swab-to-test assays. The application of these VLPs as external positive controls was therefore validated in a clinical setting, and the designed RP detection region adequately provided internal control binding sites for both RT-LAMP and RT-qPCR. In some embodiments, a minimum of 10⁵ copies of VLPs (Qβ-1P-C19L, Qβ-2P-C19L, CCMV-C19L) is required for optimal performance as a positive control in the swab-to-test assay. In some embodiments, VLPs used as a positive control in the swab-to-test assay is at least about 10⁵ copies, such as at least about 10⁶ VLPs, at least about 10⁷ VLPs, at least about 10⁸ VLPs, at least about 10⁹ VLPs, at least about 10¹⁰ VLPs, at least about 10¹¹ VLPs, or at least about 10¹² VLPs. In some embodiments, the VLP amount is provides as copies per 1 μL of sample used in one reaction.

### Recombinant Polynucleotides

Provided herein is a recombinant polynucleotide comprising, or consisting essentially of, or yet consisting of: a first polynucleotide comprising, or consisting essentially of, or yet further consisting of a QB hairpin loop with affinity for the QB coat proteins; a second polynucleotide encoding a SARS-CoV-2 N1 region; a third polynucleotide encoding a SARS-CoV-2 N2 region; and a fourth polynucleotide encoding a human RNase P region and an optional fifth polynucleotide encoding a capsid protein, e.g., QB or CCMV capsid protein.

In some embodiments, a recombinant polynucleotide as disclosed herein comprises, or consists essentially of, or yet consists of: a first polynucleotide comprising, or further consisting essentially of, or yet further consisting of a QB hairpin loop with affinity for (such as capable of binding to) the QB coat proteins; a second polynucleotide encoding a SARS-CoV-2 N1 region; a third polynucleotide encoding a SARS-CoV-2 N2 region; and a fourth polynucleotide encoding a human RNase P region. In some embodiments, the recombinant polynucleotide further comprises a fifth polynucleotide encoding a capsid protein, e.g., QB or CCMV capsid protein.

In one aspect, the first polynucleotide comprises, or consists essentially of, or yet further consists of the sequence: AATTGCATGTCTAACAGACGATCTTC (SEQ ID NO: 29) or an equivalent thereof. In one aspect, the second polynucleotide comprises, or consists essentially of, or yet further consists of the sequence: AAAATGTCTG ATAATGGACC CCAAAATCAG CGAAATGCAC CCCGCATTAC GTTTGGTGGA CCCTCAGATT CAACTGGCAG TAACCAGAAT GGAGAACGCA GTGGGGCGCG ATCAAAACAA CGTCGGCCCC AAGGTTTACC CAATAATACT GCGTCTTGGT TCACCGCTCT CAC (nt 47 to nt 219 of SEQ ID NO: 1), or an equivalent thereof. In a further aspect, the third polynucleotide comprises, or consists essentially of, or yet further consists of the sequence: CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC (nt 220 to nt 359 of SEQ ID NO: 1), or an equivalent thereof. In a further aspect, the fourth polynucleotide comprises, or consists essentially of, or yet further consists of the sequence: ATGGGACTTC AGCATGGCGG TGTTTGCAGA TTTGGACCTG CGAGCGGGTT CTGACCTGAA GGCTCTGCGC GGACTTGTGG AGACAGCCGC TCACCTTGGC TATTCAGTTG TTGCTATCAA TCATATCGTT GACTTTAAGG AAAAGAAACA GGAAATTGAA AAACCAGTAG CTGTTTCTGA ACTCTTCACA ACTTTGCCAA TTGTACAGGG AAAATCAAGA CCAATTAAAA TTTTAACTAG ATTAACAATT ATTGTCTCGG ATCCATCTCA CTGCAATGTT (nt 360 to nt 639 of SEQ ID NO: 1), or an equivalent thereof.

In one aspect, provided is a recombinant polynucleotide comprising, or alternatively consisting essentially of, or yet further consisting of: (a) a first polynucleotide comprising, or alternatively consisting essentially of, or yet further consisting of a bacteriophage Qβ (QB) hairpin loop with affinity for the QB coat protein (CP); (b) any one or any two or all three of: (i) a second polynucleotide encoding a SARS-CoV-2 N1 or a fragment thereof, (ii) a third polynucleotide encoding a SARS-CoV-2 N2 or a fragment thereof, or (iii) a fourth polynucleotide encoding a human RNase P (RP) or a fragment thereof. In further embodiments, the recombinant polynucleotide further comprises (c) an optional fifth polynucleotide encoding the QB CP.

In another aspect, provided is a recombinant polynucleotide comprising, or alternatively consisting essentially of, or yet further consisting of: (b) any one or any two or all three of: (i) a second polynucleotide encoding a SARS-CoV-2 N1 or a fragment thereof, (ii) a third polynucleotide encoding a SARS-CoV-2 N2 or a fragment thereof; (iii) a fourth polynucleotide encoding a human RNase P (RP) or a fragment thereof, and (c) a fifth polynucleotide encoding a coat protein of a cowpea chlorotic mottle virus (CCMV). In further embodiments, the recombinant polynucleotide further comprises (a) aa first polynucleotide comprising, or alternatively consisting essentially of, or yet further consisting of a bacteriophage Qβ (QB) hairpin loop with affinity for the QB coat protein (CP).

In some embodiments, a recombinant polynucleotide as disclosed herein can be encapsidated in a virus-like particle VLP and serves as a positive control in a diagnostic method. In further embodiments, the diagnostic method detects the presence or quantify the amount of a target sequence comprising, or consisting essentially of or yet further consisting of a virus-specific polynucleotide, such as a viral genome, a fragment thereof, or a polynucleotide encoded thereby or reverse-transcribed therefrom. In yet further embodiments, the virus is a coronavirus. In some embodiments, the virus is SARS-CoV-2. Accordingly, the coding sequence(s) of the recombinant polynucleotide can be determined based on the virus to be detected and the primers (and optionally probes) used in the diagnostic method. In some embodiments, the primers (and the probe if used) hybridize to a coding sequence of the recombinant polynucleotide similar to the primers hybridizing to the target sequence. For example, the relative location among the regions of the coding sequences hybridizing to the primers (and the probe if used) is substantively the same to the relative location among the regions of the target sequence hybridizing to the primers (and the probe if used). In further embodiments, a relative location between two regions of a polynucleotide comprises, or consists essentially of, or yet further consists one or more of the following: (1) in the embodiments that at least a portion of the polynucleotide is double stranded, the two regions are located in the same strand of the polynucleotide or different strands; (2) one of the two regions is on the 5′ side of (i.e., upstream to) the other region, or vice versa; (3) numbers of nucleotide residues between the two regions, or (4) physical distance between the two regions in the polynucleotide. In yet further embodiments, the primers (and the probe if use) can hybridize to a coding sequence of the recombinant polynucleotide at a level substantively similar to the level of the primers hybridizing to the target sequence.

As illustrated above, SARS-CoV-2 N1 and N2 are recommended by the FDA and CDC to be used as target sequences detected by a diagnostic method for SARS-CoV-2. Accordingly, (i) the polynucleotide encoding a SARS-CoV-2 N1 or a fragment thereof (also referred to herein as a SARS-CoV-2 N1 region or N1 region) or (ii) the polynucleotide encoding a SARS-CoV-2 N2 or a fragment thereof (also referred to herein as a SARS-CoV-2 N2 region or N2 region) or both (i) and (ii) are exemplified as a suitable coding sequence of a recombinant polynucleotide as disclosed herein. It would be understood by one of skill in the art that the present disclosure intends to include other suitable coding sequences, such as a coding sequence comprising, or consisting essentially of, or yet further consisting of a different target sequence (such as a viral genome, a fragment thereof, or a polynucleotide encoded thereby or reverse-transcribed therefrom) of SARS-CoV-2 or another virus, or an equivalent of the target sequence having substantively the same relative location of the primer/probe-hybridizing regions. Thus, those suitable coding sequences can substitute one or both of the polynucleotide encoding a SARS-CoV-2 N1 or a fragment thereof as disclosed herein, or the polynucleotide encoding a SARS-CoV-2 N2 or a fragment thereof as disclosed herein.

Additionally or alternatively, the polynucleotide encoding a human RNase P (RP) or a fragment thereof (also referred to herein as an RP region) as disclosed herein is detected or quantified in the diagnostic method in a manner similar to the polynucleotide encoding a SARS-CoV-2 N1 or a fragment thereof as disclosed herein, or the polynucleotide encoding a SARS-CoV-2 N2 or a fragment thereof, or both, but using RP specific primers (and RP specific probes). Thus, a positive result showing the RP region of the recombinant polynucleotide is detected or quantified indicates the reagents and steps of the diagnostic method function as designed. Additionally, the biological sample to be detected by the diagnostic method also has been shown as comprising the RP region. Therefore, a positive result showing the RP region is detected or quantified in the biological sample indicates polynucleotides in the sample at the time of detection is suitable for detection or quantification by the diagnostic method, for example, the sample were collected or stored properly to preserve its polynucleotides.

Accordingly, a suitable polynucleotide can be used to substitute the polynucleotide encoding a human RNase P (RP) or a fragment thereof (also referred to herein as an RP region) as disclosed herein. In some embodiments, such substitute polynucleotide can be detected or quantified using the diagnostic method. In further embodiments, such substitute polynucleotide (or an equivalent thereof comprising the same primer/probe-hybridizing regions in a substantively similar relative location) is present in a biological sample. Non-limiting examples include β-actin, 18S ribosomal RNA (RRN18S), Glyceraldehyde-3-phosphate dehydrogenase (GAPDH), Phosphoglycerate kinase 1 (PGK1), Peptidylprolyl isomerase A (PPIA), Ribosomal protein L13a (RPL13A), Ribosomal protein, large, P0 (RPLP0), Acidic ribosomal phosphoprotein P0 (ARBP), Beta-2-microglobulin (B2M), Tyrosine 3-monooxygenase/tryptophan5-monooxygenase activation protein, zeta polypeptide (YWHAZ), Succinate dehydrogenase complex, subunit A, flavoprotein (Fp) (SDHA), Transferrin receptor (TFRC), Glucuronidase, beta (GUSB), Hydroxymethylbilane synthase (HMBS), Hypoxanthine phosphoribosyltransferase 1 (HPRT1), TATA box binding protein (BP), or a fragment thereof.

In some embodiments, the first polynucleotide comprises, or alternatively consists essentially of, or yet further consists of any one of: AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1), or an equivalent thereof.

In some embodiments, the second polynucleotide comprises, or alternatively consists essentially of, or yet further consists of AAAATGTCTGATAATGGACCCCAAAATCAGCGAAATGCACCCCGCATTACGTTT GGTGGACCCTCAGATTCAACTGGCAGTAACCAGAATGGAGAACGCAGTGGGGCG CGATCAAAACAACGTCGGCCCCAAGGTTTACCCAATAATACTGCGTCTTGGTTCA CCGCTCTCAC (nt 47 to nt 219 of SEQ ID NO: 1), or an equivalent thereof.

In some embodiments, the third polynucleotide comprises, or alternatively consists essentially of, or yet further consists of any one of: CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC (nt 220 to nt 359 of SEQ ID NO: 1), GTAACACAAG CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC ATTGGCATGG AAGTCACACC TTCGGGAACG TGGTTGACCT ACACAGGTGC CATCAAATTG GATGACAAAG ATCCAAATTT CAAAGATCAA (nt 226 to nt 465 of SEQ ID NO: 2), or an equivalent of each thereof.

In some embodiments, the fourth polynucleotide comprises, or alternatively consists essentially of, or yet further consists of any one of: ATGGGACTTC AGCATGGCGG TGTTTGCAGA TTTGGACCTG CGAGCGGGTT CTGACCTGAA GGCTCTGCGC GGACTTGTGG AGACAGCCGC TCACCTTGGC TATTCAGTTG TTGCTATCAA TCATATCGTT GACTTTAAGG AAAAGAAACA GGAAATTGAA AAACCAGTAG CTGTTTCTGA ACTCTTCACA ACTTTGCCAA TTGTACAGGG AAAATCAAGA CCAATTAAAA TTTTAACTAG ATTAACAATT ATTGTCTCGG ATCCATCTCA CTGCAATGTT (nt 360 to nt 639 of SEQ ID NO: 1), CTTGTTGATG AGCTGGAGCC AGAGACCGAC ACACGGGAGC CACTGACTCG GATCCGCAAC AACTCAGCCA TCCACATCCG AGTCTTCAGG GTCACACCCA AGTAATTGAA AAGACACTCC TCCACTTATC CCCTCCGTGA TATGGCTCTT CGCATGCTGA GTACTGGACC TCGGACCAGA GCCATGTAAG AAAAGGCCTG TTCCCTGGAA GCCCAAAGGA CTCTGCATTG AGGGTGGGGG TAATTGTCTC TTGGTGGGCC CAGTTAGTGG GCCTTCCTGA GTGTGTGTAT GCGGTCTGTA ACTATTGCCA TATAAATAAA AAATCCTGTT GCACTAGTGT CCTGCCATCC (nt 472 to nt 821 of SEQ ID NO: 2), CTTGTTGATG AGCTGGAGCC AGAGACCGAC ACACGGGAGC CACTGACTCG GATCCGCAAC AACTCAGCCA TCCACATCCG AGTCTTCAGG GTCACACCCA AGTAATTGAA AAGACACTCC TCCACTTATC CCCTCCGTGA TATGGCTCTT CGCATGCTGA GTACTGGACC TCGGACCAGA GCCATGTAAG AAAAGGCCTG TTCCCTGGAA GCCCAAAGGA CTCTGCATTG AGGGTG (nt 472 to nt 707 of SEQ ID NO: 2), or an equivalent of each thereof.

In some embodiments, a recombinant polynucleotide as disclosed herein further comprises a promoter. In further embodiments, the promoter comprises, or alternatively consists essentially of, or yet further consists of a pol II promoter directing initiation of transcription catalyzed by an RNA polymerase II. In yet further embodiments, the promoter comprises, or alternatively consists essentially of, or yet further consists of a T7 promoter or an equivalent or mutant thereof. In some embodiments, the promoter is located 5′ to the first polynucleotide if present and all of the coding sequences of (b). In some embodiments, the promoter comprises, or alternatively consists essentially of, or yet further consists of a T3 promoter or an SP6 promoter. In some embodiments, the T7 promoter comprises, or alternatively consists essentially of, or yet further consists of TAATACGACTCACTATAGGG (nt 1 to nt 20 of SEQ ID NO: 1), or an equivalent thereof. In some embodiments, the SP6 promoter comprises, or alternatively consists essentially of, or yet further consists of ATTTAGGTGACACTATAG (SEQ ID NO: 32), or an equivalent thereof. In some embodiments, the SP6 promoter comprises, or alternatively consists essentially of, or yet further consists of CATACGATTTAGGTGACACTATAG (SEQ ID NO: 33), or an equivalent thereof. In some embodiments, the T3 promoter comprises, or alternatively consists essentially of, or yet further consists of AATTAACCCTCACTAAAG (SEQ ID NO: 34), or an equivalent thereof.

In some embodiments, a recombinant polynucleotide further comprises a terminator. In further embodiments, the terminator directs termination of the transcription catalyzed by the RNA polymerase II. In yet further embodiments, the terminator comprises, or alternatively consists essentially of, or yet further consists of a T7 terminator or an equivalent of mutant thereof. In some embodiments, the terminator is located 3′ to the first polynucleotide if present and all of the coding sequences. In some embodiments, the T7 terminator comprises, or alternatively consists essentially of, or yet further consists of CTA GCA TAA CCC CTT GGG GCC TCT AAA CGG GTC TTG AGG GGT TTT TTG (nt 640 to nt 687 of SEQ ID NO: 1), or an equivalent thereof.

In some embodiments, a recombinant polynucleotide as disclosed herein comprises, or alternatively consists essentially of, or yet further consist of, from 5′ to 3′, a promoter, (a), (b), and a terminator.

In some embodiments, a recombinant polynucleotide as disclosed herein further comprises a regulatory sequence directing the expression of the polynucleotide of (c) if present. In further embodiments, the regulatory sequences comprises, or alternatively consists essentially of, or yet further consists of a promoter or an enhancer or both.

In some embodiments, a recombinant polynucleotide as disclosed herein comprises, or alternatively consists essentially of, or yet further consists of any one or more of SEQ ID NO: 1, SEQ ID NO: 2, a complementary sequence of each thereof, or an equivalent of each thereof.

For CCMV, the negatively charged polynucleotide interacts with the CCMV capsid protein, specifically with the highly positively charged, arginine-rich binding domains at the N-terminus (amino acids 9-19; sequence: TRAQRRAAARK, SEQ ID NO: 3) for encapsidation (Pretto et al; and Annamalai et al). Hence, RNA of any length—in some embodiments, varying from 140 to 12,000 nt—can be packaged completely by CCMV CP as long as the protein/RNA mass ratio is as large as 6:1. In some embodiments, for sufficiently short lengths (<2,000 nt), the RNA is packaged into T=2 and T=3 capsids with one to four molecules of RNA in the capsid. In some embodiments, for sufficiently large RNAs (>4,500 nt), single molecules of RNA are packaged by two or more T=3 or T=4 capsids. In some embodiments, for intermediate lengths, single RNAs are packaged, as with wildtype length, into T=3 capsids. These scenarios arise from the capsid curvature preferred by the CP and from the relative magnitudes of the CP-RNA and CP-CP interactions. In some embodiments, In vitro self-assembly of single capsids of CCMV having RNAs of ˜3,000 nt is reminiscent of the in vivo scenario (Cadena-Nava et al.). For QP, wild type Qβ has genomic RNA with 4220 nt in length and requires Qβ hairpin to direct for encapsulation. See, for example, Fang et al., 2017; and Fang et al., 2018. In some embodiments, polynucleotide with length around 4220 nt can be preferable to be packaged by Qβ VLP.

Accordingly, in some embodiments, the recombinant polynucleotide as disclosed herein comprises, or alternatively consists essentially of, or yet further consists of about 140 to about 12000 nucleotide residues including any subranges or numbers therein, such as about 140 to about 2,000 nucleotide residues, about 140 to about 4,500 nucleotide residues, about 2,000 to about 4,500 nucleotide residues, about 500-1000 nucleotide residues, about 600-9000 nucleotide residues, about 700 nucleotide residues, or about 800 nucleotide residues. In some embodiments, the recombinant polynucleotide as disclosed herein comprises, or alternatively consists essentially of, or yet further consists of less than about 5000 nucleotide residues including any subranges or numbers therein, such as less than about 4000, less than about 3000, less than about 2000, less than about 1000, about 500-1000, about 600-9000, about 700, or about 800 nucleotide residues.

In a further aspect, provided is a double stranded polynucleotide comprising, or alternatively consisting essentially of, or yet further consisting of a recombinant polynucleotide as disclosed herein and a polynucleotide complementary thereto.

In some embodiments, the polynucleotides of (b) are cloned out-of-frame with the open reading frame to avoid protein translation of truncated SARS-CoV-1 N1 and N2 gene segments. Additionally or alternatively, any one or more of a polynucleotide as disclosed herein, a vector as disclosed herein, or a system as disclosed herein does not comprise (i.e., lack) a ribosome binding site upstream of the polynucleotides of (b). In some embodiments, the ribosome binding site is deleted. 10.1981 In one embodiment, the recombinant polynucleotide further comprises one or more regulatory sequences, e.g., a promoter or a terminator sequence or both. In one aspect, the regulatory polynucleotide comprises, or consists essentially of, or yet further consists of a promoter, optionally a pol II promoter, further optionally a T7 promoter, or an equivalent or mutant thereof, optionally located 5′ to the first polynucleotide. Mutant T7 promoters are known in the art, see, e.g., Ikeda et al. (1992) Nucl. Acid Res. Vol. 20(10):2517-2524. In one aspect, the equivalent of the T7 promoter is selected from a T3 promoter or an SP6 promoter. These sequences are known to those of skill in the art. In one embodiment, the promoter comprises, or consists essentially of, or yet further consists of TAA TAC GAC TCA CTA TAG GG (nt 1 to nt 20 of SEQ ID NO: 1), or an equivalent thereof.

In one aspect, the recombinant polynucleotide further comprises a terminator polynucleotide, e.g., a T7 terminator polynucleotide. In one aspect, the regulatory polynucleotide comprises, or alternatively consists essentially of, or yet further consists of a terminator polynucleotide, e.g., a T7 terminator polynucleotide. Non-limiting examples of such are known in the art, e.g., CTA GCA TAA CCC CTT GGG GCC TCT AAA CGG GTC TTG AGG GGT TTT TTG (nt 640 to nt 687 of SEQ ID NO: 1), or an equivalent thereof.

This disclosure also provides the recombinant polynucleotide comprising, or alternatively consisting essentially of, or yet further consisting of the sequence shown in FIG. 3, its complement (i.e., complementary sequence) or an equivalent of each thereof.

Further provided is a double stranded polynucleotide comprising, or alternatively consisting essentially of, or yet further consisting of the recombinant polynucleotide as described herein and the respective complement (i.e., complementary sequence).

In one embodiment, the polynucleotides are cloned out-of-frame with the open reading frame to avoid protein translation of truncated SARS-CoV-1 N1 and N2 gene segments. In another embodiment, the ribosome binding site upstream of the first polynucleotide has been deleted. In a yet further aspect, the polynucleotide further comprises a polynucleotide encoding a capsid protein (also referred to herein as a coat protein), e.g., QB or CCMV capsid protein (also referred to herein as QB or CCMV coat protein).

In a further aspect, the polynucleotides are detectably labeled. In some embodiments, the polynucleotides further comprises a detectable label or a selection marker or both.

### Vectors

Also provided is a vector comprising, or alternatively consisting essentially of, or yet further consisting of: the polynucleotide as described herein. Non-limiting examples of such vectors include a viral vector or a plasmid vector. In one aspect, the vector comprises or is a plasmid. Non-limiting examples of such vector is pCDFDuet plasmid or pET-28a(+), or an equivalent of each thereof.

In one aspect, provided is a vector comprising, or alternatively consisting essentially of, or yet further consisting of: a polynucleotide as disclosed herein, or a polynucleotide complementary thereto, or the double stranded polynucleotide as disclosed herein.

In some embodiments, the vector is a viral vector. In some embodiments, the vector is derived from a cowpea chlorotic mottle virus (CCMV) or a bacteriophage Qβ (QB).

In some embodiments, the vector is a non-viral vector. In further embodiments, the vector comprises, or alternatively consists essentially of, or yet further consists of a plasmid. In yet further embodiments, the plasmid is derived from any one of pCDFDuet, pET-28a(+), or an equivalent of each thereof.

In some embodiments, the polynucleotide of (b) in the vector is out-of-frame and does not express any one of an N1, an N2, an RP, or a fragment of each thereof.

In some embodiments, the polynucleotide of (c) if present in the vector is in frame and expresses the coat protein.

In some embodiments, a vector as disclosed herein lacks a ribosome binding site (RBS) upstream to the polynucleotide of (b).

In some embodiments, the polynucleotides of (b) are cloned out-of-frame with the open reading frame to avoid protein translation of truncated SARS-CoV-1 N1 and N2 gene segments.

In some embodiments, any one or more of: a polynucleotide as disclosed herein, a vector as disclosed herein, or a system as disclosed herein does not comprise (i.e., lacks) a ribosome binding site upstream of the polynucleotides of (b). In some embodiments, the ribosome binding site upstream of the polynucleotides of (b) has been deleted.

### Systems

Also provided herein is a system comprising, or alternatively consisting essentially of, or yet further consisting of a first vector comprising, or alternatively consisting essentially of, or yet further consisting of a first polynucleotide comprising, or alternatively consisting essentially of, or yet further consisting of a QB hairpin loop with affinity for the QB coat protein and a second vector comprising, or alternatively consisting essentially of, or yet further consisting of a second polynucleotide encoding a SARS-CoV-2 N1 region; a third polynucleotide encoding a SARS-CoV-2 N2 region; and a fourth polynucleotide encoding a human RNase P region. In further embodiments, the second vector further comprises a fifth polynucleotide encoding a capsid protein, e.g., QB or CCMV capsid protein.

Also provided herein is a system comprising, or alternatively consisting essentially of, or yet further consisting of a first vector comprising, or alternatively consisting essentially of, or yet further consisting of a first polynucleotide comprising, or alternatively consisting essentially of, or yet further consisting of a QB hairpin loop with affinity for the QB coat protein, a second polynucleotide encoding a SARS-CoV-2 N1 region; a third polynucleotide encoding a SARS-CoV-2 N2 region; and a fourth polynucleotide encoding a human RNase P region, and a second vector comprising, or alternatively consisting essentially of, or yet further consisting of a fifth polynucleotide encoding a capsid protein, e.g., QB or CCMV capsid protein.

In one aspect, the first polynucleotide comprises, or consists essentially of, or yet further consists of the sequence: AATTGCATGTCTAACAGACGATCTTC (as disclosed in FIG. 3) or an equivalent thereof. In one aspect, the second polynucleotide comprises, or consists essentially of, or yet further consists of the sequence: AAAATGTCTG ATAATGGACC CCAAAATCAG CGAAATGCAC CCCGCATTAC GTTTGGTGGA CCCTCAGATT CAACTGGCAG TAACCAGAAT GGAGAACGCA GTGGGGCGCG ATCAAAACAA CGTCGGCCCC AAGGTTTACC CAATAATACT GCGTCTTGGT TCACCGCTCT CAC (nt 47 to nt 219 of SEQ ID NO: 1) as shown in FIG. 3, or an equivalent thereof. In a further aspect, the third polynucleotide comprises, or consists essentially of, or yet further consists of the sequence: CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC (nt 220 to nt 359 of SEQ ID NO: 1) as shown in FIG. 3, or an equivalent thereof. In a further aspect, the fourth polynucleotide comprises, or consists essentially of, or yet further consists of the sequence: ATGGGACTTC AGCATGGCGG TGTTTGCAGA TTTGGACCTG CGAGCGGGTT CTGACCTGAA GGCTCTGCGC GGACTTGTGG AGACAGCCGC TCACCTTGGC TATTCAGTTG TTGCTATCAA TCATATCGTT GACTTTAAGG AAAAGAAACA GGAAATTGAA AAACCAGTAG CTGTTTCTGA ACTCTTCACA ACTTTGCCAA TTGTACAGGG AAAATCAAGA CCAATTAAAA TTTTAACTAG ATTAACAATT ATTGTCTCGG ATCCATCTCA CTGCAATGTT (nt 360 to nt 639 of SEQ ID NO: 1) as shown in FIG. 3, or an equivalent thereof.

In one aspect, provided is a system comprising, or alternatively consisting essentially of, or yet further consisting of (1) a first vector comprising, or alternatively consisting essentially of, or yet further consisting of (b) any one or any two or all three of: (i) a second polynucleotide encoding a SARS-CoV-2 N1 or a fragment thereof or a polynucleotide complementary thereto, (ii) a third polynucleotide encoding a SARS-CoV-2 N2 or a fragment thereof or a polynucleotide complementary thereto, or (iii) a fourth polynucleotide encoding a human RNase P (RP) or a fragment thereof or a polynucleotide complementary thereto; and (2) a second vector comprising, or alternatively consisting essentially of, or yet further consisting of: (c) a fifth polynucleotide encoding a coat protein or a polynucleotide complementary thereto. In some embodiments, the first vector further comprises (a) an optional first polynucleotide comprising, or alternatively consisting essentially of, or yet further consisting of a bacteriophage Qβ (QB) hairpin loop with affinity for the QB coat protein (CP), or a polynucleotide complementary thereto. Suitable substitutions of the coding polynucleotides are discussed in the section titled Recombinant Polynucleotides and incorporated herein by reference in its entirety.

In some embodiments, the first polynucleotide comprises, or alternatively consists essentially of, or yet further consists of any one of: AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1), or an equivalent thereof.

In some embodiments, the second polynucleotide comprises, or alternatively consists essentially of, or yet further consists of AAAATGTCTGATAATGGACCCCAAAATCAGCGAAATGCACCCCGCATTACGTTT GGTGGACCCTCAGATTCAACTGGCAGTAACCAGAATGGAGAACGCAGTGGGGCG CGATCAAAACAACGTCGGCCCCAAGGTTTACCCAATAATACTGCGTCTTGGTTCA CCGCTCTCAC (nt 47 to nt 219 of SEQ ID NO: 1), or an equivalent thereof.

In some embodiments, the third polynucleotide comprises, or alternatively consists essentially of, or yet further consists of any one of: CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC (nt 220 to nt 359 of SEQ ID NO: 1), GTAACACAAG CTTTCGGCAG ACGTGGTCCA GAACAAACCC AAGGAAATTT TGGGGACCAG GAACTAATCA GACAAGGAAC TGATTACAAA CATTGGCCGC AAATTGCACA ATTTGCCCCC AGCGCTTCAG CGTTCTTCGG AATGTCGCGC ATTGGCATGG AAGTCACACC TTCGGGAACG TGGTTGACCT ACACAGGTGC CATCAAATTG GATGACAAAG ATCCAAATTT CAAAGATCAA (nt 226 to nt 465 of SEQ ID NO: 2), or an equivalent of each thereof.

In some embodiments, the fourth polynucleotide comprises, or alternatively consists essentially of, or yet further consists of any one of: ATGGGACTTC AGCATGGCGG TGTTTGCAGA TTTGGACCTG CGAGCGGGTT CTGACCTGAA GGCTCTGCGC GGACTTGTGG AGACAGCCGC TCACCTTGGC TATTCAGTTG TTGCTATCAA TCATATCGTT GACTTTAAGG AAAAGAAACA GGAAATTGAA AAACCAGTAG CTGTTTCTGA ACTCTTCACA ACTTTGCCAA TTGTACAGGG AAAATCAAGA CCAATTAAAA TTTTAACTAG ATTAACAATT ATTGTCTCGG ATCCATCTCA CTGCAATGTT (nt 360 to nt 639 of SEQ ID NO: 1), CTTGTTGATG AGCTGGAGCC AGAGACCGAC ACACGGGAGC CACTGACTCG GATCCGCAAC AACTCAGCCA TCCACATCCG AGTCTTCAGG GTCACACCCA AGTAATTGAA AAGACACTCC TCCACTTATC CCCTCCGTGA TATGGCTCTT CGCATGCTGA GTACTGGACC TCGGACCAGA GCCATGTAAG AAAAGGCCTG TTCCCTGGAA GCCCAAAGGA CTCTGCATTG AGGGTGGGGG TAATTGTCTC TTGGTGGGCC CAGTTAGTGG GCCTTCCTGA GTGTGTGTAT GCGGTCTGTA ACTATTGCCA TATAAATAAA AAATCCTGTT GCACTAGTGT CCTGCCATCC (nt 472 to nt 821 of SEQ ID NO: 2), CTTGTTGATG AGCTGGAGCC AGAGACCGAC ACACGGGAGC CACTGACTCG GATCCGCAAC AACTCAGCCA TCCACATCCG AGTCTTCAGG GTCACACCCA AGTAATTGAA AAGACACTCC TCCACTTATC CCCTCCGTGA TATGGCTCTT CGCATGCTGA GTACTGGACC TCGGACCAGA GCCATGTAAG AAAAGGCCTG TTCCCTGGAA GCCCAAAGGA CTCTGCATTG AGGGTG (nt 472 to nt 707 of SEQ ID NO: 2), or an equivalent of each thereof.

In some embodiments, the first vector further comprises a promoter. In further embodiments, the promoter comprises, or alternatively consists essentially of, or yet further consists of a pol II promoter directing initiation of transcription catalyzed by an RNA polymerase II. In yet further embodiments, the promoter comprises, or alternatively consists essentially of, or yet further consists of a T7 promoter, or an equivalent or mutant thereof. In some embodiments, the promotor is located 5′ to the first polynucleotide if present and all of the coding sequences. In some embodiments, the promoter comprises, or consists essentially of, or yet further consists of a T3 promoter or an SP6 promoter. In some embodiments, the T7 promoter comprises, or alternatively consists essentially of, or yet further consist of TAATACGACTCACTATAGGG (nt 1 to nt 20 of SEQ ID NO: 1), or an equivalent thereof.

In some embodiments, the first vector further comprises a terminator. In further embodiments, the terminator directs termination of the transcription catalyzed by the RNA polymerase II. In some embodiments, the terminator comprises, or alternatively consists essentially of, or yet further consists of a T7 terminator or an equivalent of mutant thereof. In some embodiments, the terminator is located 3′ to the first polynucleotide if present and all of the coding sequences. In some embodiments, the T7 terminator comprises, or alternatively consists essentially of, or yet further consists of CTA GCA TAA CCC CTT GGG GCC TCT AAA CGG GTC TTG AGG GGT TTT TTG (nt 640 to nt 687 of SEQ ID NO: 1), or an equivalent thereof.

In some embodiments, the first vector comprises, or alternatively consists essentially of, or yet further consists of: from 5′ to 3′, a promoter, (a) if present, (b) and a terminator.

In some embodiments, the first vector comprises, or alternatively consists essentially of, or yet further consists of any one of: SEQ ID NO: 1, SEQ ID NO: 2, a complementary sequence of each thereof, or an equivalent of each thereof.

In some embodiments, the promoter is about 140 to about 12,000 (including any subranges or numbers therebetween) nucleotides upstream to the terminator. In further embodiments, the promoter is about 140 to about 2,000 nucleotides, or about 140 to about 4,500 nucleotides, or about 2,000 to about 4,500 nucleotides, or about 500-1000 nucleotides, or about 600-9000 nucleotides, or about 700 nucleotides, or about 800 nucleotides upstream to the terminator. In yet further embodiments, the coat protein is a CCMV coat protein.

In some embodiments, the promoter is less than about 5000 (including any subranges or numbers therein) nucleotides upstream to the terminator. In further embodiments, the promoter is less than about 4000, or less than about 3000, or less than about 2000, or less than about 1000, or about 500-1000, or about 600-9000, or about 700, or about 800 nucleotides upstream to the terminator. In yet further embodiments, the coat protein is a QP coat protein.

In some embodiments, the second vector further comprises a regulatory sequence operatively linked to the polynucleotide of (c) and directing the expression of the coat protein. In further embodiments, the regulatory sequence comprises a promoter, an enhancer, or both.

In some embodiments, either or both of the vectors is a viral vector. Additionally or alternatively, either or both of the vectors is a non-viral vector. In further embodiments, the non-viral vector comprises, or alternatively consists essentially of, or yet further consists of a plasmid. In yet further embodiments, the plasmid is derived from pCDFDuet, or pET-28a(+), or an equivalent of each thereof.

In some embodiments, the first vector is derived from pET-28a(+) and comprises, or alternatively consists essentially of, or yet further consists of SEQ ID NO: 1, or SEQ ID NO: 2, or an equivalent of each thereof.

In some embodiments, an equivalent to a polynucleotide as set forth with a SEQ ID NO comprises, or consists essentially of, or yet consists of the polynucleotide as set forth in the SEQ ID NO and having one or more T nucleotide residues substituted with a U nucleotide residue. In further embodiments, an equivalent to a polynucleotide as set forth with a SEQ ID NO comprises, or consists essentially of, or yet consists of the polynucleotide as set forth in the SEQ ID NO and having each of its T nucleotide residues substituted with a U nucleotide residue.

In some embodiments, the second vector is derived from pCDFDuet.

In some embodiments, the polynucleotide of (b) in the first vector is out-of-frame and does not express any one of an N1, an N2, an RP, or a fragment of each thereof.

In some embodiments, the first vector lacks a ribosome binding site (RBS) upstream to the polynucleotide of (b).

In some embodiments, the polynucleotide of (c) in the second vector is in frame and express the coat protein.

In some embodiments, the polynucleotides of (b) are cloned out-of-frame with the open reading frame to avoid protein translation of truncated SARS-CoV-1 N1 and N2 gene segments.

In some embodiments, the ribosome binding site upstream of the polynucleotides of (b) has been deleted.

In one embodiment, the recombinant polynucleotide further comprises one or more regulatory sequences, e.g., a promoter or a terminator sequence or both. In one aspect, the regulatory polynucleotide comprises, or consists essentially of, or yet further consists of a promoter, optionally a pol II promoter, and further optionally a T7 promoter. In one embodiment, the recombinant polynucleotide further comprises a T7 promoter, or an equivalent or mutant thereof located 5′ to the first polynucleotide. In one embodiment, the regulatory sequence comprises, or alternatively consists essentially of, or yet further consists of a T7 promoter, or an equivalent or mutant thereof optionally located 5′ to the first polynucleotide. Mutant T7 promoters are known in the art, see, e.g., Ikeda et al. (1992) Nucl. Acid Res. Vol. 20(10):2517-2524. In one aspect, the equivalent of the T7 promoter is selected from a T3 promoter or an SP6 promoter. These sequences are known to those of skill in the art. In one embodiment, the promoter comprises, or consists essentially of, or yet further consists of TAA TAC GAC TCA CTA TAG GG (nt 1 to nt 20 of SEQ ID NO: 1) as shown in FIG. 3, or an equivalent thereof.

In one aspect, the recombinant polynucleotide further comprises a terminator polynucleotide, optionally a T7 terminator polynucleotide. In one aspect, the regulatory sequence comprises, or alternatively consists essentially of, or yet further consists of a terminator polynucleotide, optionally a T7 terminator polynucleotide. Non-limiting examples of such are known in the art, e.g., CTA GCA TAA CCC CTT GGG GCC TCT AAA CGG GTC TTG AGG GGT TTT TTG (nt 640 to nt 687 of SEQ ID NO: 1) as shown in FIG. 3, or an equivalent thereof.

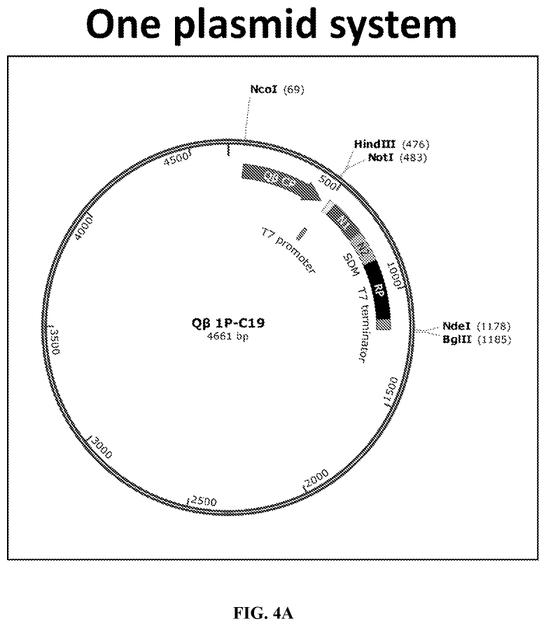

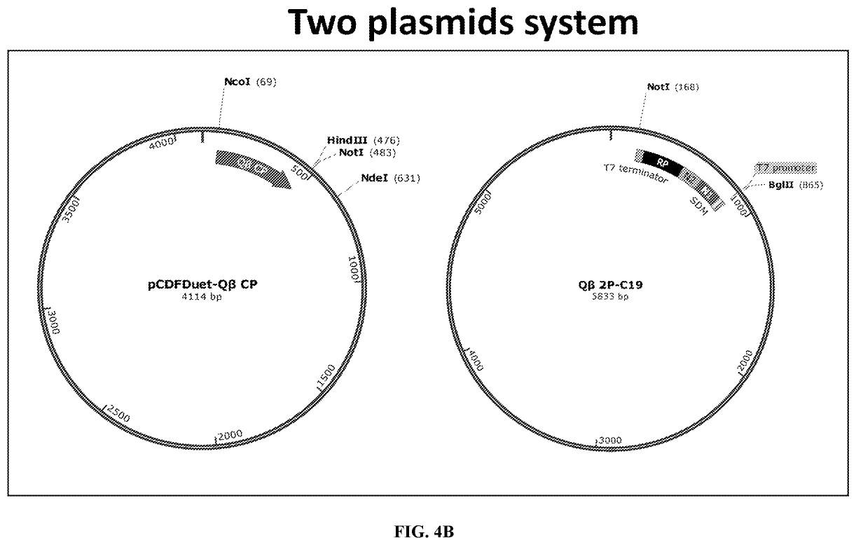

Also provided is a system as shown in FIG. 4 and described herein, or an equivalent of each thereof.

In one embodiment, the polynucleotides are cloned out-of-frame with the open reading frame to avoid protein translation of truncated SARS-CoV-1 N1 and N2 gene segments. In another embodiment, the ribosome binding site upstream of the first polynucleotide has been deleted. In a yet further aspect, the polynucleotide further comprises a polynucleotide encoding a capsid protein, e.g., QB or CCMV capsid protein.

In a further aspect, the vectors and/or the polynucleotides are detectably labeled. In some embodiments, the vectors and/or the polynucleotides further comprises a detectable label or a selection marker or both.

In one aspect, the vector includes a viral vector or a plasmid vector. In one aspect, the vector comprises, or alternatively consists essentially of, or yet further consists of, or is a plasmid. Non-limiting examples of such vector is pCDFDuet-QB plasmid, or pET-28a(+) or an equivalent of each thereof.

### Particles

In a further aspect, provided herein is a recombinant viral particle comprising, or alternatively consisting essentially of, or yet further consisting of an RNA polynucleotide comprising, or alternatively consisting essentially of, or yet further consisting of a first polynucleotide comprising, or alternatively consisting essentially of, or yet further consisting of a QB hairpin loop with affinity for the QB coat proteins; a second polynucleotide encoding a SARS-CoV-2N1 region; a third polynucleotide encoding a SARS-CoV-2N2 region; and a fourth polynucleotide encoding a human RNase P region. In one aspect, the recombinant viral particle further comprises a capsid protein, e.g., QB or CCMV capsid protein.

VLPs were developed from bacteriophage Qβ or plant virus CCMV as biomimetic nanotechnology for use as positive control probes for COVID-19 diagnostic assays. Qβ and CCMV offer several advantages over current approaches that use either bacteriophage MS2 VLPs (Asuragen, see, e.g., Asuragen Develops Armored RNA Quant® SARS-CoV-2 Control as cited herein) and replication-defective mammalian virus (SeraCare). See, e.g., AccuPlex, Virus Materials| SeraCare available at www.seracare.com/resources-and-education/accuplex-recombinant-virus-materials/, last accessed Jul. 15, 2020. First, the Qβ capsid is 5 Å larger and contains approximately 20% more genomic RNA compared to MS2. See, e.g., Golmohammadi et al., Structure. 1996 May 15; 4(5):543-54. Therefore, Qβ can accommodate a higher payload; and indeed Qβ can package 100× copies of target RNA compared to MS2 (Yao et al.). Furthermore, despite extensive sequence identity between Qβ and MS2, the coat protein subunits of Qβ offer higher thermal stability compared to MS2; the Qβ capsid gains thermal stability based on inter-subunit disulfide bonds. See, e.g., Ashcroft et al., J Nanosci Nanotechnol. 2005 December; 5(12):2034-41. Previous studies comparing Qβ and MS2 with comparable RNA cargo have shown that Qβ is more stable over a range of temperatures (−20° C., 4° C., and 45° C., see, e.g., Yao et al.). Taking together, data suggest that Qβ would be a better candidate for long term storage or ambient shipping.

To the best of Applicant's knowledge, CCMV has never been reported for development and application of positive control in molecular diagnostic assays. A particular advantage of the CCMV system is the straightforward in vitro reconstitution—hence offering a high degree of modularity. Purified coat proteins could be stored and reconstituted around a target RNA cassette as needed, for example, if mutants or new strains emerge or adapt to the probe for use in other diagnostic assays. CCMV coat proteins could be obtained through heterologous expression (see, e.g., Diaz-Valle et al., J Virol Methods. 2015 Dec. 1; 225:23-9) or through molecular farming in plants. The latter offers a high degree of scalability and speed, and can be implemented with relatively non-sophisticated infrastructure while keeping manufacturing costs low. See, e.g., Rybicki. Wiley Interdiscip Rev Nanomed Nanobiotechnol. 2020 March; 12(2):e1587.

Furthermore, the bacteriophage and plant VLPs (Qβ and CCMV) offer advantages compared to a replication-deficient mammalian virus, which always carries a risk of residual activity or reversion, which may pose safety risks. See, e.g., Meyerson and Sawyer. Trends Microbiol. 2011 June; 19(6):286-94; and Hanley K A. Evolution (N Y). 2011 December; 4(4):635-643. Qβ and CCMV are non-infectious to mammals and unable to replicate in mammalian cells (see, e.g., Wen and Steinmetz. Chem Soc Rev. 2016 Jul. 25; 45(15):4074-126); therefore offering another layer of safety in particular for use in low-resource settings where sterile and biosafety facilities are not always attainable. Moreover, production of Qβ and CCMV through fermentation or molecular farming is more cost effective and higher yielding compared to manufacture of mammalian virus in mammalian cell cultures. See, e.g., Ibrahim et al. Front Plant Sci. 2019 Jun. 19; 10:803.

In one aspect, provided is a virus-like particle (VLP) comprising, or alternatively consisting essentially of, or yet further consisting of a polynucleotide encapsidated in a coat protein (CP). In some embodiments, the polynucleotide comprises, or consists essentially of, or yet further consists of an RNA. In further embodiments, the polynucleotide is an RNA.

Additionally or alternatively, the polynucleotide comprises, or alternatively consists essentially of, or yet further consists of (b) any one or any two or all three of: (i) a second polynucleotide encoding a SARS-CoV-2 N1 or a fragment thereof or a polynucleotide complementary thereto, (ii) a third polynucleotide encoding a SARS-CoV-2 N2 or a fragment thereof or a polynucleotide complementary thereto, or (iii) a fourth polynucleotide encoding a human RNase P (RP) or a fragment thereof or a polynucleotide complementary thereto. In further embodiments, the polynucleotide further comprises (a) a first polynucleotide comprising, or alternatively consisting essentially of, or yet further consisting of a bacteriophage Qβ (QB) hairpin loop with affinity for the QB coat protein (CP), or a polynucleotide complementary thereto.

Suitable substitutions of the coding polynucleotides are discussed in the section titled Recombinant Polynucleotides and incorporated herein by reference in its entirety.

In some embodiments, the VLP is derived from a bacteriophage Qβ (QB) and the CP comprises, or alternatively consists essentially of, or yet further consists of a QB coat protein.

In some embodiments, the VLP is derived from a cowpea chlorotic mottle virus (CCMV) and the CP comprises, or alternatively consists essentially of, or yet further consists of a CCMV coat protein.

In some embodiments, the polynucleotide, such as the RNA polynucleotide, comprises, or alternatively consists essentially of, or yet further consists of the first polynucleotide of (a); the second polynucleotide of (b)(i); the third polynucleotide of (b)(ii); and the fourth polynucleotide of (b)(iii).

In some embodiments, the VLP comprises, or alternatively consists essentially of, or yet further consists of at least about 10⁸ copies of the polynucleotide (such as an RNA polynucleotide) from every μg of VLPs, including any subranges or number therein, for example, at least about 10⁹ copies μg of VLPs, or at least about 10¹⁰ copies μg of VLPs, or at least about 10¹¹ copies μg of VLPs, or at least about 10¹² copies μg of VLPs, or at least about 10¹³ copies μg of VLPs, or at least about 10¹⁴ copies μg of VLPs, or at least about 10¹⁵ copies μg of VLPs, or at least about 10¹⁶ copies μg of VLPs, or more.

In some embodiments, the first polynucleotide, the second polynucleotide, the third polynucleotide, and the fourth polynucleotide are as disclosed herein. In some embodiments, the encapsidated polynucleotide comprises, or consists essentially of, or yet further consists of any one of SEQ ID NO: 1, SEQ ID NO: 2, a complementary sequence of each thereof, or an equivalent of each thereof. In some embodiments, the encapsidated polynucleotide is an RNA polynucleotide.

In some embodiments, the equivalent of a polynucleotide comprises, or consists essentially of, or yet further consists of the polynucleotide having each of its T nucleotide residues substituted with a U nucleotide residue.

In a further aspect, provided is a method of preparing a cowpea chlorotic mottle virus (CCMV). The method comprises, or alternatively consists essentially of, or yet further consists of a CCMV coat protein and a polynucleotide (such as an RNA polynucleotide) that comprises, or alternatively consists essentially of, or yet further consists of (b) any one or any two or all three of: (i) a second polynucleotide encoding a SARS-CoV-2 N1 or a fragment thereof or a polynucleotide complementary thereto, (ii) a third polynucleotide encoding a SARS-CoV-2 N2 or a fragment thereof or a polynucleotide complementary thereto, or (iii) a fourth polynucleotide encoding a human RNase P (RP) or a fragment thereof or a polynucleotide complementary thereto. In further embodiments, the polynucleotide, such as the RNA polynucleotide further comprises (a) a first polynucleotide comprising, or alternatively consisting essentially of, or yet further consisting of a bacteriophage Qβ (QB) hairpin loop with affinity for the QB coat protein (CP), or a polynucleotide complementary thereto.

The method comprises, or alternatively consists essentially of, or yet further consists of mixing the polynucleotide (such as the RNA polynucleotide) with a CCMV coat protein in vitro, thereby the CCMV coat protein and the polynucleotide are self-assembled to a VLP. In some embodiments, the CCMV coat protein is isolated from a CCMV produced by a CCMV infected plant cell. In some embodiments, the method further comprises isolating the VLP, i.e., the CCMV-derived VLP comprising the polynucleotide (such as the RNA polynucleotide). In further embodiments, the CCMV-derived VLP comprises, or alternatively consists essentially of, or yet further consists of the polynucleotide (such as the RNA polynucleotide) encapsidated in a coat protein.

In yet a further aspect, provided is a method of preparing a VLP as disclosed herein. The method comprises, or alternatively consists essentially of, or yet further consists of culturing a host cell as disclosed herein under conditions suitable for producing the VLP. In some embodiments, the method further comprises isolating the VLP.

### Host Cells

Also provided herein is a host cell comprising the recombinant polynucleotide, vector or system as disclosed herein. In one aspect, the cell is a eukaryotic cell or a prokaryotic cell, e.g., a prokaryotic cell such as for example, E. coli.

In one aspect, provided is a host cell comprising any one or more of: a polynucleotide as disclosed herein, a vector as disclosed herein, or a system as disclosed herein.

In some embodiments, the cell is a eukaryotic cell. In further embodiments, the cell is a plant cell. In yet further embodiments, the cell is a Vigna unguiculate cell or a California black-eyed peas No. 5 cell.

In some embodiments, the cell is a prokaryotic cell. In further embodiments, the cell is an E. coli. In yet further embodiments, the cell is a BL21 (DE3) E. coli cell.

In a further aspect, provided is a method of preparing any one or more of: a recombinant polynucleotide as disclosed herein, a vector as disclosed herein, or a system as disclosed herein. In some embodiments, the method comprises, or alternatively consists essentially of, or yet further consists of growing a host cell as disclosed herein under conditions to replicate the recombinant polynucleotide or the vector or the system. In some embodiments, the method comprises, or alternatively consists essentially of, or yet further consists of growing a host cell as disclosed herein under conditions to express (such as transcribe) a polynucleotide or a vector or a system optionally encoding the recombinant polynucleotide. In some embodiments, the method further comprises isolating the any one or more of the recombinant polynucleotide, the vector, or the system.

### Methods

Also provided herein is method of preparing a recombinant RNA polynucleotide described herein, comprising, or alternatively consisting essentially of, or consisting of, growing or culturing the host cell as described herein under conditions to replicate or express the polynucleotide. In one aspect, the RNA polynucleotide is isolated. Alternatively, recombinant expression technology can be used to replicate the polynucleotide.

In one aspect, provided is a method for detecting a SARS-CoV-2 infection in a subject in need thereof. The method comprises, or alternatively consists essentially of, or yet further consists of (1) contacting a sample suspected of comprising the SARS-CoV-2 and isolated from the subject with a probe that directly or indirectly generates a detectable signal when contacting with any one or any two or all three of: (i) a second polynucleotide encoding a SARS-CoV-2 N1 or a fragment thereof, (ii) a third polynucleotide encoding a SARS-CoV-2 N2 or a fragment thereof, or (iii) a fourth polynucleotide encoding a human RNase P (RP) or a fragment thereof, (2) separately contacting a VLP as disclosed herein with the probe; and (3) measuring the detectable signal of the contacted sample of (1) and the detectable signal of the contacted VLP of (2).

In some embodiments, the measured signal of the contacted sample of (1) at a level substantially similar or higher than the measured signal of the contacted VLP of (2) indicates the sample comprises SARS-CoV-2. In further embodiments, the measured signal of the contacted sample of (1) at a level substantially similar or higher than the measured signal of the contacted VLP of (2) indicates the subject has a SARS-CoV-2 infection.

In some embodiments, the measured signal of the sample of (1) contacting with any one or both of: (i) a second polynucleotide encoding a SARS-CoV-2 N1 or a fragment thereof, (ii) a third polynucleotide encoding a SARS-CoV-2 N2 or a fragment thereof, at a level substantially similar or higher than the measured signal of the VLP of (2) contacting with any one or both of: (i) a second polynucleotide encoding a SARS-CoV-2 N1 or a fragment thereof, (ii) a third polynucleotide encoding a SARS-CoV-2 N2 or a fragment thereof indicates the sample comprises SARS-CoV-2. In further embodiments, the measured signal of the sample of (1) contacting with any one or both of: (i) a second polynucleotide encoding a SARS-CoV-2 N1 or a fragment thereof, (ii) a third polynucleotide encoding a SARS-CoV-2 N2 or a fragment thereof, at a level substantially similar or higher than the measured signal of the VLP of (2) contacting with any one or both of: (i) a second polynucleotide encoding a SARS-CoV-2 N1 or a fragment thereof, (ii) a third polynucleotide encoding a SARS-CoV-2 N2 or a fragment thereof indicates the subject has a SARS-CoV-2 infection.

In some embodiments, the measured signal of the sample of (1) contacting with (iii) a fourth polynucleotide encoding a human RNase P (RP) or a fragment thereof at a level substantially similar or higher than the measured signal of the VLP of (2) contacting with (iii) a fourth polynucleotide encoding a human RNase P (RP) or a fragment thereof indicates the sample is suitable to be detected by the method, e.g., the sample was collected and stored properly and comprises materials within the sensitivity range of the method.

Suitable substitutions of the coding polynucleotides are discussed in the section titled Recombinant Polynucleotides and incorporated herein by reference in its entirety.

In some embodiments, the method comprises, or consists essentially of, or yet further consists of a reverse transcription polymerase chain reaction (RT-PCR), or a reverse transcription loop-mediated isothermal amplification (RT-LAMP). In further embodiments, the RT-PCR is a quantitative RT-PCR or a droplet digital PCR (ddPCR). In further embodiments, the RT-LAMP is a swab-to-test assay. In yet further embodiments, the SARS-CoV-2 and the VLP are lysed, for example, by heat. In yet further embodiments, the heating step comprises, or consists essentially of, or yet further consists of incubating the sample or the VLP at about 95° C. for at last about 5 minutes.

In some embodiments, the VLP is derived from Qβ and in the contacting step, the VLP concentration is at least about 10⁵ VLPs per μL, such as at least about 10⁶ VLPs per μL, at least about 10⁷ VLPs per μL, at least about 10⁸ VLPs per μL, at least about 10⁹ VLPs per μL, at least about 10¹⁰ VLPs per μL, at least about 10¹¹ VLPs per μL, or at least about 10¹² VLPs per μL. In some embodiments, the VLP concentration as disclosed herein is used when the method comprises, or consists essentially of, or yet further consists of a swab-to-test assay.

Additionally or alternatively, the VLP is derived from Qβ and in the contacting step, the VLP concentration is at least about 10-100 pg VLP per μL, such as at least 10 pg VLP per μL, at least 20 pg VLP per μL, at least 30 pg VLP per μL, at least 40 pg VLP per μL, at least 50 pg VLP per μL, at least 60 pg VLP per μL, at least 70 pg VLP per μL, at least 80 pg VLP per μL, at least 90 pg VLP per μL, at least 100 pg VLP per μL, at least 110 pg VLP per μL, at least 120 pg VLP per μL, at least 130 pg VLP per μL, at least 140 pg VLP per μL, at least 150 pg VLP per μL, at least 160 pg VLP per μL, at least 170 pg VLP per μL, at least 180 pg VLP per μL, at least 190 pg VLP per μL, at least 200 pg VLP per μL, at least 250 pg VLP per μL, at least 300 pg VLP per μL, at least 350 pg VLP per μL, at least 400 pg VLP per μL, at least 450 pg VLP per μL, or at least 500 pg VLP per μL. In some embodiments, the VLP concentration as disclosed herein is used when the method comprises, or consists essentially of, or yet further consists of an RT-PCR or an RT-LAMP or both.

In some embodiments, the VLP is derived from CCMV and in the contacting step, the VLP concentration is at least about 10⁴ VLPs per μL, such as at least about 10⁵ VLPs per μL, at least about 10⁶ VLPs per μL, at least about 10⁷ VLPs per μL, at least about 10⁸ VLPs per μL, at least about 10⁹ VLPs per μL, at least about 10¹⁰ VLPs per μL, at least about 10¹¹ VLPs per μL, or at least about 10¹² VLPs per pL. In some embodiments, the VLP concentration as disclosed herein is used when the method comprises, or consists essentially of, or yet further consists of a swab-to-test assay.

Additionally or alternatively, the VLP is derived from CCMV and in the contacting step, the VLP concentration is at least about 10-100 pg VLP per μL, such as at least 10 pg VLP per μL, at least 20 pg VLP per μL, at least 30 pg VLP per μL, at least 40 pg VLP per μL, at least 50 pg VLP per μL, at least 60 pg VLP per μL, at least 70 pg VLP per μL, at least 80 pg VLP per μL, at least 90 pg VLP per μL, at least 100 pg VLP per μL, at least 110 pg VLP per μL, at least 120 pg VLP per μL, at least 130 pg VLP per μL, at least 140 pg VLP per μL, at least 150 pg VLP per μL, at least 160 pg VLP per μL, at least 170 pg VLP per μL, at least 180 pg VLP per μL, at least 190 pg VLP per μL, at least 200 pg VLP per μL, at least 250 pg VLP per μL, at least 300 pg VLP per μL, at least 350 pg VLP per μL, at least 400 pg VLP per μL, at least 450 pg VLP per μL, or at least 500 pg VLP per μL. In some embodiments, the VLP concentration as disclosed herein is used when the method comprises, or consists essentially of, or yet further consists of an RT-PCR or an RT-LAMP or both.

In some embodiments, the VLP concertation/amount is provided as 1 μL of sample (such as the biological sample) to be used in one reaction.

In some embodiments, the method further comprises administering to the subject having a SARS-CoV-2 infection a therapy to treat COVID-19.

In some embodiments, coat proteins (CPs) of the VLP are cross linked. In further embodiments, the cross linking is intra particle. In yet further embodiments, the cross-linking comprises, or consists essentially of, or yet further consists of a homobifunctional cross-linker 3, 3′-dithiobis (sulfosuccinimidylpropionate) (DTSSP) between lysine side chains of adjacent CPs.

In some embodiments, the Qβ derived VLP is stabilized. In further embodiments, pores of the VLP is plugged, optionally through chemistry or through freeze-drying in excipients.

In some embodiments, a method as disclosed herein further comprises contacting a no template control (NTC) with the primers (and probe is used), serving as a negative control. In some embodiments, the NTC does not comprise one or more of a coding sequence as disclosed herein, according, it should not show a detectable signal when detected or quantified by a method as disclosed herein, and a detectable signal indicates a false positive and the sample is contaminated.

In some embodiments, different amounts of a VLP as disclosed can be used in a method as disclosed herein to establish a correlation (such as a positive linear correlation) between strength of the detectable signal and the VLP amount (which can be converted to copies of coding sequences). Accordingly, based on the established correlation, the detectable signal measured in the biological sample can be converted to copies of target sequences, and thus, the SARS-CoV-2 amount in the sample can be quantified. Therefore, a method as disclosed herein can be used to detect the presence of a SARS-CoV-2 in a biological sample, as well as to quantify the amount of SARS-CoV-2 in the biological sample.

In some embodiments, the detectable signal is colorimetric. For example, phenol red in the RT-LAMP reagent changes in color from pink to yellow due to the accumulation of H+ ions during amplification. Accordingly, such color change can be detected and indicates amplification of any one or any two or all three of: (i) a second polynucleotide encoding a SARS-CoV-2 N1 or a fragment thereof, (ii) a third polynucleotide encoding a SARS-CoV-2 N2 or a fragment thereof, or (iii) a fourth polynucleotide encoding a human RNase P (RP) or a fragment thereof.

Further provided are methods for detecting COVID-19 comprising contacting a sample suspected of comprising COVID-19 with a testing agent as known in the art and described herein. A separate sample comprises, or alternatively consists essentially of, or yet further consists of the recombinant viral particle as described herein. The viral particle acts as a positive control and can be a component of an assay described in Table 1. In one aspect, the assay or detection method further comprises PCR or LAMP. If a subject is determined to be positive for COVID-19 infection, the method can further comprise administering to the subject a therapy to treat COVID-19. Non-limiting examples of such are provided in Table 2, below. In some embodiments, the therapy to treat COVID-19 comprises, or consists essentially of, or yet further consists of one or more of: an anti-viral agent, optionally remdesivir, lopinavir, ritonavir, ivermectin, tamiflu, or favipiravir; an anti-inflammatory agent, optionally dexamethasone, tocilizumab, kevzara, colcrys, hydroxychloroquine, chloroquine, or a kinase inhibitor; a covalescent plasma from a subject recovered from a SARS-CoV-2 infection; an antibody binding to SARS-CoV-2, optionally bamlanivimab, etesevimab, casirivimab, or imdevimab; or an antibiotic agent, optionally azithromycin. In one aspect, a therapeutically effective amount is administered.

TABLE 2
Method of Treating SARS-COV-2 Infection.

| | | | | Clinical Trial |
|---|---|---|---|---|
| Intervention | Type | Comparator | Phase | Registration # |
| Hydroxychloroquine | Antimalarial | Placebo | Ph | ChiCTR2000029559 |
| | | | IV | |
| Hydroxychloroquine | Antimalarial | Supportive care | Ph | ChiCTR2000029740 |
| | | | IV | |
| Sildenafil citrate | Vasodilator | Single arm | Ph | NCT04304313 |
| | | | III | |
| α lipoic acid | Antioxidant | Placebo | Ph | ChiCTR2000029851 |
| | | | IV | |
| Ebastine + | Mucolytic; | Lopinavir + | Ph | ChiCTR2000030535 |
| Lopinavir + | antiviral | Interferon α | IV | |
| Interferon α | | | | |
| Mesenchymal stem | Cell therapy | Placebo | Ph | ChiCTR2000029990 |
| cells | | | I/II | |
| Danoprevir + | Antiviral; | Lopinavir/ritonavir; | Ph | NCT04291729 |
| Ritonavir + | Immunomodulator | Pegasys; | IV | |
| interferon | | Novaferon; | | |
| | | Chinese medicines + | | |
| | | interferon (4 | | |
| | | arms) | | |
| Bevacizumab | Antibody | Single arm | Ph | NCT04275414 |
| biosimilar | | | II/III | |
| Tocilizumab | Antibody | Single arm | Ph | NCT04315480 |
| | | | II | |
| Remdesivir | Antiviral | Placebo | Ph | NCT04257656 |
| | | | III | |
| Remdesivir | Antiviral | Placebo | Ph | NCT04252664 |
| | | | III | |
| Dipyridamole | Vasodilator | Supportive care | Ph | ChiCTR2000030055 |
| | | | IV | |
| Low molecular | Anticoagulant | Supportive care | Ph | ChiCTR2000030946 |
| weight heparin | | | IV | |
| Bromhexine + | Mucolytic; | Arbidol | N/A | NCT04273763 |
| Arbidol umifenovir + | Antiviral; | umifenovir + | | |
| Favipiravir + | Immunomodulator | Interferon α2b | | |
| Interferon α2b | | | | |
| Favipiravir high | Antiviral | N/A | Ph | ChiCTR2000029996 |
| dose; middle dose; | | | II | |
| low dose (3 arms) | | | | |
| Levamisole + | Antiparasitic; | Lopinavir + | Ph | NCT04331470 |
| Budesonide + | Corticosteroid; | Ritonavir + | II/III | |
| Formoterol + | Bronchodilator; | Hydoxychloroquine | | |
| Lopinavir + | Antiviral; | | | |
| Ritonavir + | Antimalarial | | | |
| Hydoxychloroquine | | | | |
| Interferon β1α + | Immunomodulator; | Hydroxychloroquine + | Ph | NCT04343768 |
| Hydroxychloroquine + | Antimalarial; | Lopinavir/Ritonavir | IV | |
| Lopinavir/Ritonavir; | Antiviral | | | |
| Interferon β1b + | | | | |
| Hydroxychloroquine + | | | | |
| Lopinavir/Ritonavir | | | | |
| (2 arms) | | | | |
| Interferon β1α + | Immunomodulator; | Lopinavir/Ritonavir + | Ph | NCT04350671 |
| Lopinavir/Ritonavir + | Antimalarial; | Hydroxychloroquine | IV | |
| Hydroxychloroquine | Antiviral | | | |
| Umifenovir + | Antiviral; | Lopinavir/Ritonavir + | Ph | NCT04350684 |
| Interferon β1a + | Immunomodulator; | Hydroxychloroquine | IV | |
| Lopinavir/Ritonavir + | Antimalarial | | | |
| Hydroxychloroquine | | | | |
| Methylprednisolone | Corticosteroid | Supportive care | Ph | NCT04244591 |
| | | | II/III | |
| DAS181 | Antiviral | Single arm | N/A | NCT04324489 |
| Hydroxychloroquine; | Antimalarial | Placebo | Ph | NCT04346667 |
| Chloroquine (3 | | | IV | |
| arms) | | | | |
| Chloroquine | Antimalarial | Placebo | Ph | ChiCTR2000031204 |
| phosphate | | | II | |
| Thalidomide | Immunomodulator | Placebo | Ph | NCT04273581 |
| | | | II | |
| Hydroxychloroquine; | Antimalarial | Placebo | Ph | NCT04351191 |
| Chloroquine (3 | | | IV | |
| arms) | | | | |
| Baricitinib; | Anti- | Antiviral and/or | Ph | NCT04320277 |
| Ritonavir | inflammatory; | hydroxychloroquine | III | |
| | Antiviral | | | |
| Danoprevir + | Antiviral | Single arm | Ph | NCT04345276 |
| Ritonavir | | | IV | |
| α lipoic acid | Antioxidant | Placebo | Ph | ChiCTR2000030471 |
| | | | IV | |
| Intravenous | Antibody | Supportive care | Ph | NCT04261426 |
| immunoglobulin | | | II/III | |
| (IVIG) | | | | |
| Hydroxychloroquine | Antimalarial | Chloroquine | Ph | ChiCTR2000029898 |
| | | phosphate | IV | |
| Hydroxychloroquine | Antimalarial | Chloroquine | Ph | ChiCTR2000029899 |
| | | phosphate | IV | |
| Bevacizumab | Antibody | Supportive care | N/A | NCT04305106 |
| biosimilar | | | | |
| Danoprevir + | Antiviral | Supportive care | N/A | ChiCTR2000030259 |
| Ritonavir | | | | |
| Tocilizumab + | Antibody | Supportive care | Ph | ChiCTR2000030580 |
| Adalimumab | | | IV | |
| PD-1 blocking | Antibody; | Supportive care | Ph | NCT04268537 |
| antibody; Thymosin | hormone | | II | |
| (2 arms) | | | | |
| Mesenchymal stem | Cell therapy | Single arm | Ph | NCT04269525 |
| cells | | | II | |
| Corticosteroid | Corticosteroid | Supportive care | Ph | ChiCTR2000030481 |
| | | | IV | |
| Anakinra | Immunomodulator | Supportive care | Ph | NCT04341584 |
| | | | II | |
| Eculizumab | Antibody | Supportive care | Ph | NCT04346797 |
| | | | II | |
| Sarilumab + | Antibody; | Sarilumab | Ph | NCT04341870 |
| Azithromycin + | Antibiotic; | | II/III | |
| Hydroxychloroquine | Antimalarial | | | |
| Methylprednisolone | Corticosteroid | Supportive care | N/A | NCT04273321 |
| Remdesivir 5 days; | Antiviral | N/A | Ph | NCT04292899 |
| 10 days (2 arms) | | | III | |
| Remdesivir 5 days; | Antiviral | Supportive care | Ph | NCT04292730 |
| 10 days (2 arms) | | | III | |
| Povidone-Iodine; | Antiseptic | Placebo | Ph | NCT04344236 |
| Chlorhexidine (2 | | | II | |
| arms) | | | | |
| Favipiravir + | Antiviral; | Favipiravir; | N/A | NCT04310228 |
| Tocilizumab | Antibody | Tocilizumab (2 | | |
| | | arms) | | |
| ASC09 + | Antiviral | Oseltamivir | Ph | NCT04261270 |
| oseltamivir; | | | III | |
| ritonavir + | | | | |
| oseltamivir (2 arms) | | | | |
| Nintedanib | Antifibrotic | Placebo | Ph | NCT04338802 |
| | | | II | |
| Tocilizumab | Antibody | Supportive care | Ph | ChiCTR2000029765 |
| | | | IV | |
| Ruxolitinib + | Anti- | Supportive care | Ph | NCT04348695 |
| Simvastatin | inflammatory; | | II | |
| | Vasodilator | | | |
| Hydroxychloroquine + | Antimalarial; | Hydroxychloroquine + | N/A | NCT04349592 |
| Azithromycin | Antibiotic | Placebo; | | |
| | | Placebo (2 arms) | | |
| Xiyanping + | Antiviral; | Lopinavir/ritonavir + | N/A | NCT04275388 |
| Lopinavir/ritonavir + | Immunomodulator | interferon α | | |
| interferon α | | | | |
| Tocilizumab + | Anti- | Supportive care | Ph | NCT04335305 |
| Pembrolizumab | inflammatory; | | II | |
| | Immune booster | | | |
| Mesenchymal stem | Cell therapy | Single arm | Ph | NCT04315987 |
| cells | | | I | |
| Siltuximab; | Anti- | N/A | Ph | NCT04329650 |
| Methylprednisolone | inflammatory | | II | |
| (2 arms) | | | | |
| Hydroxychloroquine | Antimalarial | Placebo | Ph | NCT04333654 |
| | | | I | |
| Interferon α1b + | Immunomodulator; | N/A | Ph | NCT04320238 |
| Thymosin a1; | hormone | | III | |
| Interferon α1b (2 | | | | |
| arms) | | | | |
| Methylprednisolone | Corticosteroid | Supportive care | Ph | NCT04323592 |
| | | | II/III | |
| Chloroquine | Antimalarial | Supportive care | Ph | ChiCTR2000029992 |
| phosphate; | | | IV | |
| Hydroxychloroquine | | | | |
| (2 arms) | | | | |
| Hydroxychloroquine; | Antimalarial; | Placebo | Ph | NCT04328272 |
| Hydroxychloroquine + | Antibiotic | | III | |
| Azithromycin (2 | | | | |
| arms) | | | | |
| Triazavirin | Antiviral | Placebo | Ph | ChiCTR2000030001 |
| | | | III | |
| Danoprevir + | Antiviral | Single arm | N/A | ChiCTR2000031734 |
| Ritonavir | | | | |
| Tocilizumab | Antibody | Supportive care | Ph | NCT04346355 |
| | | | II | |
| Angiotensin 1-7 | Vasodilator | Placebo | Ph | NCT04332666 |
| | | | II/III | |
| Colchicine | Anti- | Standard of care | Ph | NCT04328480 |
| | inflammatory | | III | |
| Thalidomide | Immunomodulator | Placebo | Ph | NCT04273529 |
| | | | II | |
| Lopinavir/ritonavir; | Antiviral | Supportive care | Ph | NCT04252885 |
| Arbidol umifenovir | | | IV | |
| (2 arms) | | | | |
| Colchicine | Anti- | Standard of care | Ph | NCT04322565 |
| | inflammatory | | II | |
| Leflunomide | Immunomodulator | Placebo | Ph | ChiCTR2000030058 |
| | | | III | |
| Interferon α1β | Immunomodulator | Supportive care | Ph | NCT04293887 |
| | | | I | |
| Chloroquine | Antimalarial | Supportive care | Ph | ChiCTR2000030718 |
| phosphate | | | | |
| Nitazoxanide | Antiviral | Placebo | N/A | NCT04348409 |
| Hydroxychloroquine + | Antimalarial; | Single arm | Ph | NCT04329572 |
| Azithromycin | Antibiotic | | I | |
| Tocilizumab | Antibody | Single arm | Ph | ChiCTR2000030196 |
| | | | II | |
| Interferon α1β | Immunomodulator | Placebo | Ph | ChiCTR2000029989 |
| Mesenchymal stem | Cell therapy | Placebo | Ph | ChiCTR2000030138 |
| cells | | | II | |
| ASC09/ritonavir; | Antiviral | N/A | N/A | NCT04261907 |
| Lopinavir/ritonavir | | | | |
| (2 arms) | | | | |
| Favipiravir + | Antiviral; | Favipiravir; | Ph | ChiCTR2000030894 |
| Tocilizumab | Antibody | Tocilizumab (2 | IV | |
| | | arms) | | |
| Mesenchymal stem | Anti- | Single arm | Ph | NCT04276987 |
| cell-derived | inflammatory | | I | |
| exosomes | | | | |
| Ulinastatin | Anti- | Supportive care | Ph | ChiCTR2000030779 |
| | inflammatory | | IV | |
| Chloroquine | Antimalarial | Single arm | Ph | ChiCTR2000029975 |
| phosphate | | | IV | |
| Chloroquine | Antimalarial | Supportive care | Ph | ChiCTR2000029988 |
| phosphate | | | IV | |
| Dexamethasone + | Corticosteroid; | Hydroxychloroquine | Ph | NCT04347980 |
| Hydroxychloroquine | Antimalarial | | III | |
| Intravenous | Antibody | Placebo | Ph | NCT04350580 |
| immunoglobulin | | | III | |
| (IVIG) | | | | |
| Ruxolitinib | Anti- | Single arm | Ph | NCT04334044 |
| | inflammatory | | I/II | |
| Tacrolimus + | Immunomodulator; | Supportive care | Ph | NCT04341038 |
| Methylprednisolone | Anti- | | III | |
| | inflammatory | | | |
| Favipiravir | Antiviral | Supportive care | N/A | NCT04333589 |
| TD-0903 (2 arms) | Anti- | Placebo | Ph | NCT04350736 |
| | inflammatory | | I | |
| Arbidol umifenovir; | Antiviral | N/A | Ph | NCT04255017 |
| Oseltamivir; | | | IV | |
| Lopinavir/ritonavir | | | | |
| (3 arms) | | | | |
| Arbidol umifenovir; | Antiviral; | Arbidol | Ph | NCT04254874 |
| peginterferon alfa- | Immunomodulator | umifenovir | | |
| 2b | | | | |
| Methylprednisolone | Corticosteroid | N/A | Ph | NCT04263402 |
| low dose; high dose | | | IV | |
| (2 arms) | | | | |
| Hydroxychloroquine | Antimalarial | Placebo | Ph | NCT04343677 |
| (3 arms) | | | II | |
| Hydroxychloroquine | Antimalarial | Placebo | Ph | NCT04346329 |
| | | | III | |
| Hydroxychloroquine | Antimalarial | Single arm | Ph | NCT04351620 |
| | | | I | |
| Luvox fluvoxamine | Immunomodulator | Placebo | Ph | NCT04342663 |
| | | | II | |
| Piclidenoson | Anti- | Supportive care | Ph | NCT04333472 |
| | inflammatory | | II | |
| Hydroxychloroquine + | Antimalarial; | Bromhexine | Ph | NCT04340349 |
| Bromhexine | Mucolytic | | I | |
| Allogeneic natural | Cell therapy | Single arm | Ph | NCT04344548 |
| killer cells | | | I/II | |
| Chloroquine analog; | Antimalarial; | Standard of care | Ph | NCT04333914 |
| Nivolumab; | Immune booster; | | II | |
| Tocilizumab (3 | Anti- | | | |
| arms) | inflammatory | | | |
| Hydroxychloroquine + | Antimalarial; | Single arm | Ph | NCT04344457 |
| Indomethacin + | Anti- | | I/II | |
| Zithromax | inflammatory; | | | |
| | Antibiotic | | | |
| Tofacitinib | Anti- | Single arm | Ph | NCT04332042 |
| | inflammatory | | II | |
| Favipiravir + | Antiviral; | Favipiravir; | Ph | ChiCTR2000030987 |
| Chloroquine | Antimalarial | Placebo (2 arms) | II/III | |
| phosphate | | | | |
| Hydroxychloroquine + | Antimalarial; | Single arm | Ph | NCT04348474 |
| Azithromycin | Antibiotic | | I | |
| Inactivated | Cell therapy | Placebo | N/A | NCT04347174 |
| Mycobacterium w | | | | |
| Interferon α1β | Immunomodulator | Supportive care | Ph | ChiCTR2000030013 |
| | | | IV | |
| Hydroxychloroquine | Antimalarial | Placebo | III | NCT04341441 |
| daily; weekly (2 | | | | |
| arms) | | | | |
| Lopinavir/ritonavir + | Antiviral | Lopinavir/ritonavir | N/A | ChiCTR2000029468 |
| emtricitabine/tenofovir | | | | |
| alafenamide | | | | |
| fumarate | | | | |
| Alvesco ciclesonide; | Antimalarial; | Standard of care | Ph | NCT04330586 |
| Alvesco ciclesonide + | Corticosteroid | | II | |
| Hydroxychloroquine | | | | |
| (2 arms) | | | | |
| Emtricitabine + | Antiviral; | Placebo | Ph | NCT04334928 |
| Tenofovir disoproxil + | Antimalarial | | III | |
| Hydroxychloroquine; | | | | |
| Emtricitabine + | | | | |
| Tenofovir | | | | |
| disoproxil; | | | | |
| Hydroxychloroquine | | | | |
| (3 arms) | | | | |
| Mesenchymal stem | Cell therapy | Placebo | Ph | NCT04339660 |
| cells | | | I/II | |
| Danoprevir + | Antiviral | Supportive care | N/A | ChiCTR2000030472 |
| Ritonavir | | | | |
| Mesenchymal stem | Cell therapy | Single arm | Ph | NCT04313322 |
| cells | | | I | |
| Novaferon + | Antiviral; | Arbidol | Ph | ChiCTR2000029573 |
| Arbidol umifenovir; | Immunomodulator | umifenovir; | IV | |
| Novaferon + | | Lopinavir/ritonavir | | |
| Lopinavir/ritonavir | | (3 arms) | | |
| (3 arms) | | | | |
| Escin oral; | Anti- | Supportive care | Ph | NCT04322344 |
| parenteral (2 arms) | inflammatory | | II/III | |
| Tradjenta linagliptin | Antiviral | Insulin | Ph | NCT04341935 |
| | | | IV | |
| Mesenchymal stem | Cell therapy | Placebo | N/A | NCT04273646 |
| cells | | | | |
| Azvudine | Antiviral | Single arm | N/A | ChiCTR2000030041 |

### Kits

Further provided is a kit comprising, or alternatively consisting essentially of, or yet further consisting of any one or more of: the polynucleotide, vector, host cell, system, VLP or recombinant viral particle as described herein and optionally, instructions for use. In some embodiments, the kit further comprises a therapy to treat COVID-19, such as one or more of those as disclosed herein.

### EXPERIMENTAL METHODS

### Example 1: VLP-Based Control Probe for SARS-CoV-2 RT-qPCR

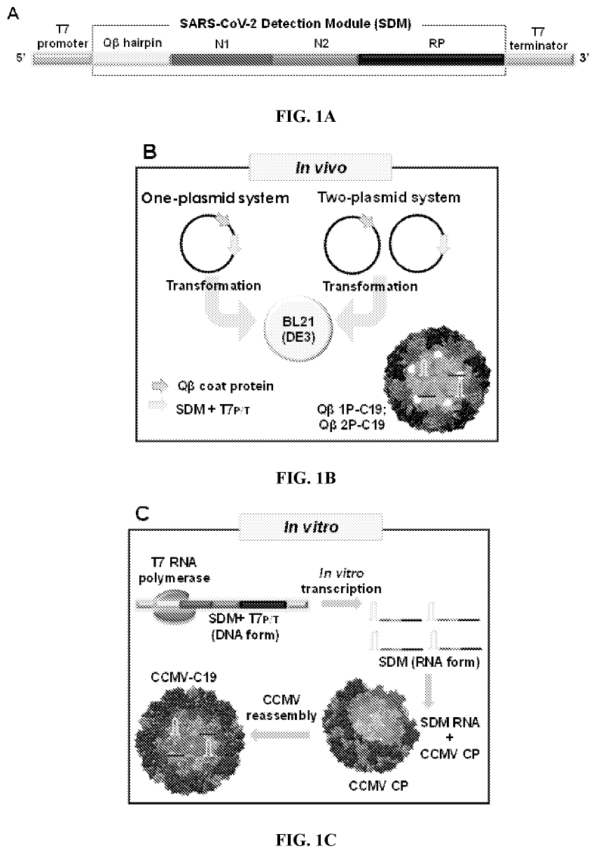

The design of the SARS-CoV-2 detection module (SDM) is shown in FIG. 1. The 622 nt SDM consists of 4 segments with the first being a 29-nt Qβ hairpin has high affinity for the Qβ coat proteins (Witherell and Uhlenbeck, Biochem., 28 (1989) 71-76). The QP hairpin loop is followed by the 3 target regions: two SARS-CoV-2 N regions (accession NC_045512.2 N1: gene location: 28271-28443; N2: gene location: 29091-29230) and the human RNase P region (or RP; accession NM_006413: gene location: 1-280). The module is flanked by a T7 promoter and T7 terminator at the 5′ and 3′ ends, respectively, for RNA transcription to enable both in vitro and in vivo reconstitution of chimeric VLPs (FIG. 1 and FIG. 3). SDM flanked with a T7 promoter and T7 terminator (SDM+T7_{P/T}) was cloned into pCDFDuet-Qβ and pET-28a (+) to generate plasmid Qβ 1P-C19 and Qβ 2P-C19, respectively (FIG. 4). Plasmid Qβ 1P-C19 allows co-expression of the Qβ coat protein (CP) gene and SDM RNA from the same vector whereas plasmid Qβ 2P-C19 expresses only SDM RNA. pCDFDuet™-Qβ, which expresses the Qβ CP gene, was co-transformed into the same bacterial cell with plasmid Qβ 2P-C19 for in vivo encapsidation of SDM RNAs. Several safety measures were built into the synthesis of these positive controls. SDM+T7_{P/T} was cloned out-of-frame with the open reading frame to avoid protein translation of truncated SARS-CoV-2 N1 and N2 gene segments. The ribosome binding site (RBS) upstream of SDM was removed for the same reasons.

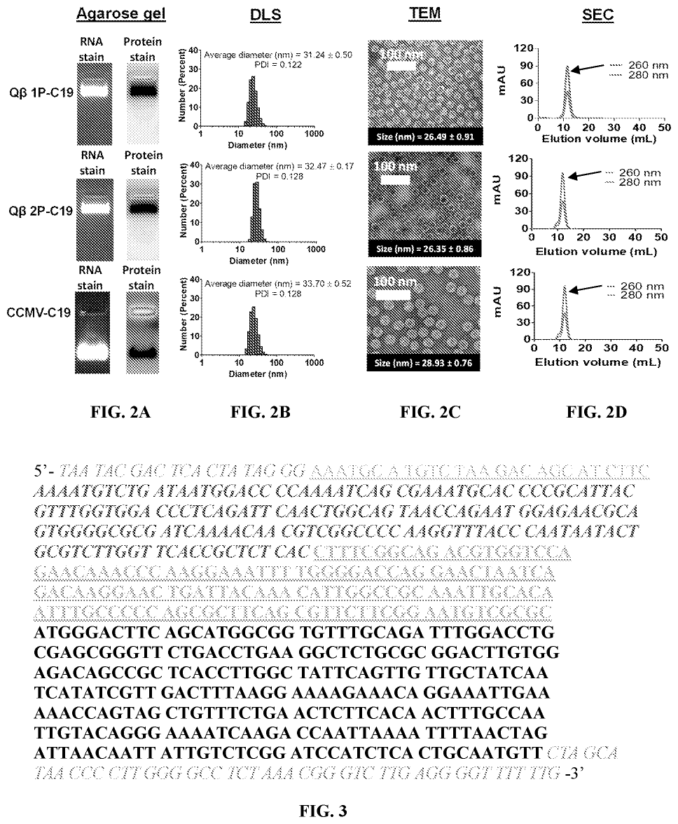

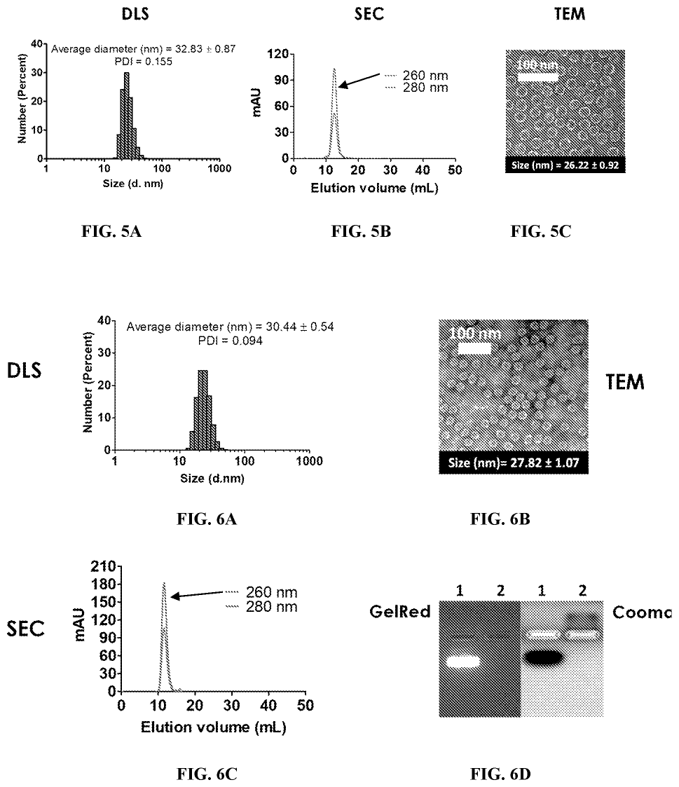

Qβ-based positive controls: In vivo encapsidation of SDM RNAs in QR VLPs was achieved by co-expression of Qβ CPs and SDM RNAs in E. coli using the aforementioned one-plasmid system and two-plasmid system to produce Qβ 1P-C19 VLPs and Qβ 2P-C19 VLPs, respectively. CCMV-based positive controls: Native CCMV was obtained from infected black-eyed pea No. 5 plants and disassembled to obtain purified CPs. The purified CPs were then reassembled with in vitro transcribed SDM RNAs (FIG. 1B). Disassembly and reassembly are achieved through careful adjustment of the buffer conditions. Characterization of the VLP-based SARS-CoV-2 RT-PCR positive controls revealed production of intact, pure, and monodisperse chimeric VLPs (FIG. 2 and Table 3). Lastly, the particle properties of the chimeric VLPs packaging the SDM matched data of Qβ VLPs devoid of the SDM (FIG. 5) as well as native CCMV particles (FIG. 6).

TABLE 3
Total nucleic acids extracted from VLP-based SARS-
COV-2 positive controls and percentage of SDM.

| | Qβ | Qβ 1P-C19 | Qβ 2P-C19 | CCMV-C19 |
|---|---|---|---|---|
| Total nucleic acids | 198.83 ± 35.09 | 301.13 ± 17.42 | 314.00 ± 70.85 | 168.24 ± 16.35 |
| (ng/μg VLP)* | | | | |
| 260/280 | 2.47 ± 0.01 | 2.39 ± 0.02 | 2.40 ± 0.01 | 2.82 ± 0.10 |
| 260/230 | 1.84 ± 0.16 | 2.26 ± 0.28 | 2.03 ± 0.21 | 1.92 ± 0.14 |
| Normalized copy | n/a | 8.78 ± 0.29 | 8.76 ± 0.23 | 10.18 ± 0.34 |
| number (10\hat{ }) | | | | |
| Percentage of SDM | n/a | 3.98 | 3.80 | 100.00 |
| RNAs in total RNAs | | | | |
| (%)** | | | | |

*Total nucleic acids include carrier RNA.
**Normalised copy number for every 5 ng of total nucleic acids was tabulated to obtain percentage of SDM RNAs in total RNAs.

### Example 2: VLP-Based Control Probe for SARS-CoV-2 RT-LAMP

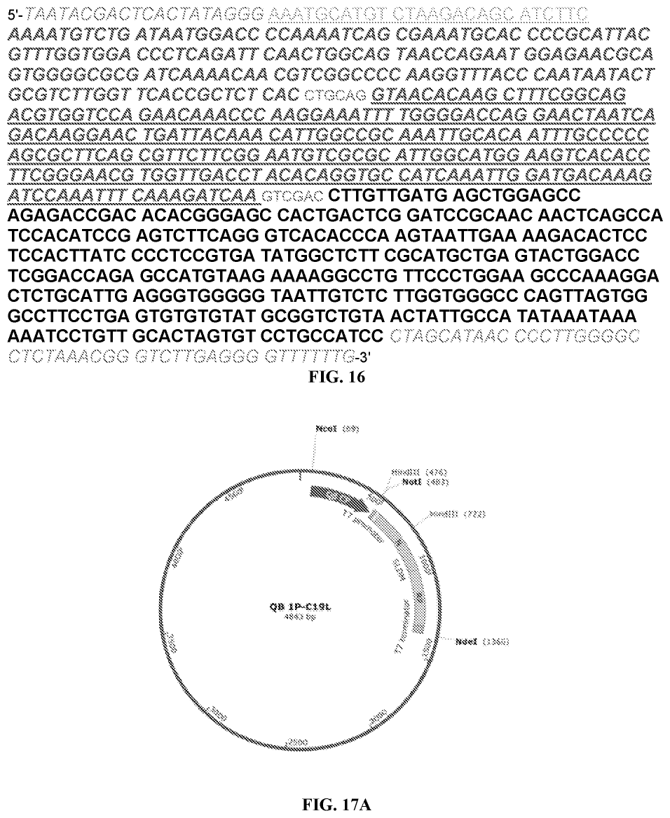

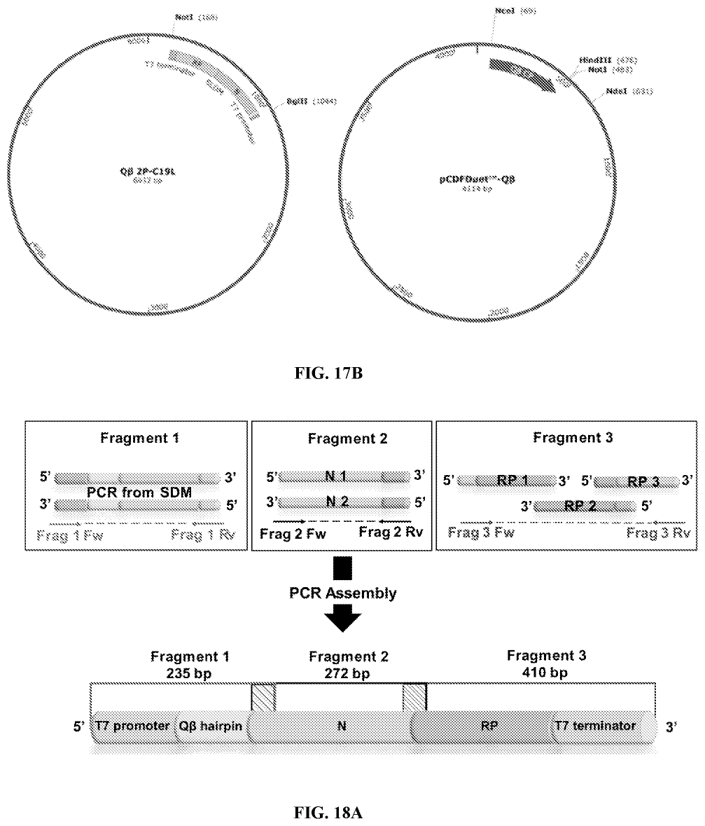

The design of the SARS-CoV-2 LAMP detection module (SLDM) is shown in FIG. 15. The 801 nt SLDM consists of 3 main segments with the first being a 29-nt Qβ hairpin has high affinity for the Qβ coat proteins (Witherell and Uhlenbeck, Biochem., 28 (1989) 71-76). The Qβ hairpin loop is followed by the 2 target regions: SARS-CoV-2 N regions (accession NC_045512.2 N1: gene location: 28271-28443, and 29081-29320) and the human RNase P region (or RP; accession NM_005837.2: gene location: 581-930). Each target region comprises binding sites for RT-qPCR's Taqman primers/probe, as well as RT-LAMP primers. Restriction sites Pst1 and SaI1 were added before and after N target region for ease of swapping genes. The module is flanked by a T7 promoter and T7 terminator at the 5′ and 3′ ends, respectively, for RNA transcription to enable both in vitro and in vivo reconstitution of chimeric VLPs (FIGS. 15-16). SLDM flanked with a T7 promoter and T7 terminator (SLDM+T7_{P/T}) was cloned into pCDFDuet-Qβ and pET-28a (+) to generate plasmid Qβ 1P-C19L and Qβ 2P-C19L, respectively (FIG. 17). Plasmid Qβ 1P-C19L allows co-expression of the Qβ coat protein (CP) gene and SLDM RNA from the same vector whereas plasmid QP 2P-C19L expresses only SLDM RNA. pCDFDuet™-Qβ, which expresses the Qβ CP gene, was co-transformed into the same bacterial cell with plasmid Qβ 2P-C19L for in vivo encapsidation of SLDM RNAs. Several safety measures were built into the synthesis of these positive controls. SLDM+T7_{P/T} was cloned out-of-frame with the open reading frame to avoid protein translation of truncated SARS-CoV-2 N gene segments. The ribosome binding site (RBS) upstream of SLDM was removed for the same reasons.

Qβ-based positive controls: In vivo encapsidation of SLDM RNAs in Qβ VLPs was achieved by co-expression of Qβ CPs and SLDM RNAs in E. coli using the aforementioned one-plasmid system and two-plasmid system to produce Qβ 1P-C19L VLPs and Qβ 2P-C19L VLPs, respectively.

CCMV-based positive controls: Native CCMV was obtained from infected black-eyed pea No. 5 plants and disassembled to obtain purified CPs. The purified CPs were then reassembled with in vitro transcribed SLDM RNAs (FIG. 15B). Disassembly and reassembly are achieved through careful adjustment of the buffer conditions. Characterization of the VLP-based SARS-CoV-2 RT-PCR positive controls revealed production of intact, pure, and monodisperse chimeric VLPs (FIG. 2 and Table 3). Lastly, the particle properties of the chimeric VLPs packaging the SLDM matched data of Qβ VLPs devoid of the SLDM (FIG. 5) as well as native CCMV particles (FIG. 6).

### Example 3: Improved Detection of COVID-19 Through Bioinspired Diagnostics

qRT-PCR (reverse transcription polymerase chain reaction) is a molecular biology technique used to detect and screen for COVID-19. While highly sensitive, qRT-PCR lacks reliable internal controls to verify negative results. Such internal, positive controls are essential for validating the assay and negating the potential presence of processing errors that preclude the enzymatic RT-PCR steps. Current controls are synthetic RNA transcripts. These “naked” RNA transcripts are unstable and prone to degradation; therefore, these molecules cannot be used reliably to spike samples under investigation. The lack of verification of negative results presents a considerable health and environmental risk. This disclosure sets out to overcome this technological hurdle through application of a unique biomaterial, specifically the use of an intrinsic positive control, in which the synthetic RNA transcripts are packaged into a plant virus capsid. nCoV virus and the plant virus cowpea chlorotic mottle virus (CCMV) are both icosahedral RNA-based viruses; therefore, the CCMV-based nanoparticle probe with synthetic RNA more closely mimics the conditions encountered by the RNA template of the nCoV within clinical or environmental samples. It is critical to note that the incorporation of synthetic nCoV fragments, which are non-replicating and noninfectious, in CCMV will not create hazardous samples. In addition to biodesigning and scaling up the production of this probe, it is designed to be used as a control by clinical qRT-PCR assay as well as a newly developed LAMP assay. The latter does not require highly specialized equipment. The LAMP assay relies on simple instruments and reagents to provide a visual, colorimetric read out under normal lighting conditions. The utility of RT-LAMP has been demonstrated for the detection of plant viruses outside of the laboratory, in the field. This positive control can then be added to each specimen prior to processing and will reveal whether any sample processing steps have resulted in the degradation of the nCoV target, which would otherwise result in a false negative read out. High accuracy in the detection assays is critical to prevent further spread of COVID-19, to appropriately allocate scarce resources to care for patients with the disease, and to understand the full epidemiological impact of this pandemic.

### Advantages

The disclosed probes have several advantages over the prior art. The Qβ capsid has a radius 5 å larger than that of the MS2 capsid. Qβ also has about 20% more genomic RNA. (Golmohammadi et al., Structure 1996, 4, 543-554.) Therefore, Qβ can encapsidate more RNA than MS2. (Yao et al., Food Environ. Virol. 2019, 11, 383-392.) In fact, Qβ can package 100 copies more RNA compared to MS2.

Despite extensive sequence identity between Qβ and MS2, coat protein subunits Qbeta has better thermal stability than MS2 due to the inter-subunit disulphide bonds. (Ashcroft et al., J. Nanosci. Nanotechnol. 2005, 5, 2034-2041.) In fact, direct comparison between Qβ VLP and MS2 VLP demonstrated Qβ VLP is more stable than MS2 at a range of temperature (−20° C., 4° C., and 45° C.) with similar RNA cargo. Hence, Qβ is a better candidate for long term storage or ambient shipping.

Applicant's results showed a one-plasmid system with indirect upstream ribosome binding site for encapsulation of target RNA within Qβ showed better packaging efficiency than two-plasmid system which is frequently reported. (Fang et al., RSC Adv. 2018, 8, 21399-21406; Fang et al., Nucleic Acids Res. 2017, 45, 3519-3527). To the best of Applicant's knowledge, the one-plasmid system for expression is novel. In addition, the CCMV as positive control is novel. Despite taking longer production time than MS2 and QP, CCMV is highly scalable for encapsulation of pure target RNA. CCMV infected leaves could be stored at −80° C. for years until use.

In vitro encapsulation of target RNA in CCMV is well established and can be easily done by adjusting buffer's pH. Also, CCMV encapsulation does not require assembly-initiation signal which is required in MS2 and QP. Purification of in vitro encapsidation of cargo in CCMV is much more convenient than MS2 or Qβ which only involves centrifugation by 100 kDa membrane filter. As opposed to MS2 (Zhang et al., 2015, 99, 7047-7057. doi.org/10.1007/s00253-015-6664-4.), which requires extra step such as precipitation with PEG and NaCl that will take additional of 2-4 h. Incubation of in vitro cargo encapsulated Qβ in hydrogen peroxide is required to promote cysteine oxidation and full encapsidation. (Herbert et al., Agents. Bioconjug. Chem. 2020, 31, 1529-1536)

In addition, the probe has advantages as compared to a replication-deficient mammalian vector. Replication-deficient mammalian virus has risk of residual activity or revert mutation which is potentially dangerous. (Meyerson et al., Trends in Microbiology. NIH Public Access June 2011, pp 286-294; Hanley et al., Evol. Educ. Outreach 2011, 4, 635-643. doi.org/10.1007/s12052-011-0365-y.) CCMV and Qβ are non-infectious to mammals and unable to replicate in mammalian cells. Hence, this would serve as extra safety layer to be used as positive control especially at low-resource settings where biosafety hood is absent or need to be handled with certain level of safety precaution. Moreover, production of CCMV and Qβ is cheaper than mammalian vector which require mammalian cells for production (Ibrahim et al., Frontiers in Plant Science. Frontiers Media S.A. May 31, 2019, p 803. doi.org/10.3389/fpls.2019.00803.) and do not require complex downstream processing/purification. This is an especially important economical consideration when large amount of positive control is needed to be manufactured at affordable cost. CCMV and Qβ do not need to helper RNA for encapsulation which is expensive and inconvenient for large-scale production. (AccuPlex™ SARS-CoV-2 Reference Material Kit. Seracare.) CCMV and Qβ are highly scalable in short amount of time compared to mammalian virus.

### Example 4: Biomimetic Virus-Like Particles as SARS-CoV-2 Positive Controls for RT-PCR Diagnostics

Positive controls for the molecular assays have been developed to validate each test and to provide high accuracy. However, most available positive controls require cold-chain distribution and cannot serve as full-process control. To overcome these shortcomings, disclosed herein is the production of biomimetic virus-like particles (VLPs) as SARS-CoV-2 positive controls. A SARS-CoV-2 detection module for RT-PCR was encapsidated into VLPs from a bacteriophage and a plant virus. The chimeric VLPs were obtained either by in vivo reconstitution and co-expression of the target detection module and coat proteins or by in vitro assembly of purified detection module RNA sequences and coat proteins. These VLP-based positive controls mimic SARS-CoV-2 packaged RNA while being non-infectious. Most importantly, it was demonstrated that the positive controls are scalable, stable, and can serve broadly as controls, from RNA extraction to PCR in clinical settings.

### Results and Discussion

Design of the SARS-CoV-2 Detection Module (SDM)

The design of the SDM was based on the CDC-recommended detection regions. The 622 nt SDM consists of 4 segments with the first being a Qβ hairpin loop. This 29-nt QP hairpin has high affinity for the Qβ coat proteins (see, e.g., Witherell and Uhlenbeck. Biochemistry. 1989 Jan. 10; 28(1):71-6) and is appended to the targeted RNA sequences to facilitate SDM encapsidation into Qβ VLPs. In previous work, the Qβ hairpin loop has been used to direct the encapsidation of protein cargos such as enzymes for biocatalytic applications (Fiedler et al., Angew Chem Int Ed Engl. 2010 Dec. 10; 49(50):9648-51) as well as target RNAs (Fang et al. 2018; and Fang et al., Nucleic Acids Res. 2017 Apr. 7; 45(6):3519-3527). The Qβ hairpin loop is followed by the 3 target regions: two SARS-CoV-2 N regions (Accession NC_045512.2 N1: gene location: 28271-28443; N2: gene location: 29091-29230) and the human RNase P region (or RP; Accession NM_006413: gene location: 1-280). The module is flanked by a T7 promoter and T7 terminator at the 5′ and 3′ ends, respectively, for RNA transcription to enable both in vitro and in vivo reconstitution of chimeric VLPs (FIGS. 1 and 3). SDM flanked with a T7 promoter and T7 terminator (SDM+T7_{P/T}) was cloned into pCDFDuet-Qβ and pET-28a (+) to generate plasmid Qβ 1P-C19 and Qβ 2P-C19, respectively (FIG. 4). Plasmid Qβ 1P-C19 allows co-expression of the Qβ coat protein (CP) gene and SDM RNA from the same vector whereas plasmid Qβ 2P-C19 expresses only SDM RNA. pCDFDuet™-Qβ, which expresses the Qβ CP gene, was co-transformed into the same bacterial cell with plasmid Qβ 2P-C19 for in vivo encapsidation of SDM RNAs. The two-plasmid system has been used successfully in previous work for in vivo reconstitution of Qβ around target RNAs. See, e.g., Fang et al., 2018 and Fang et al., 2017.

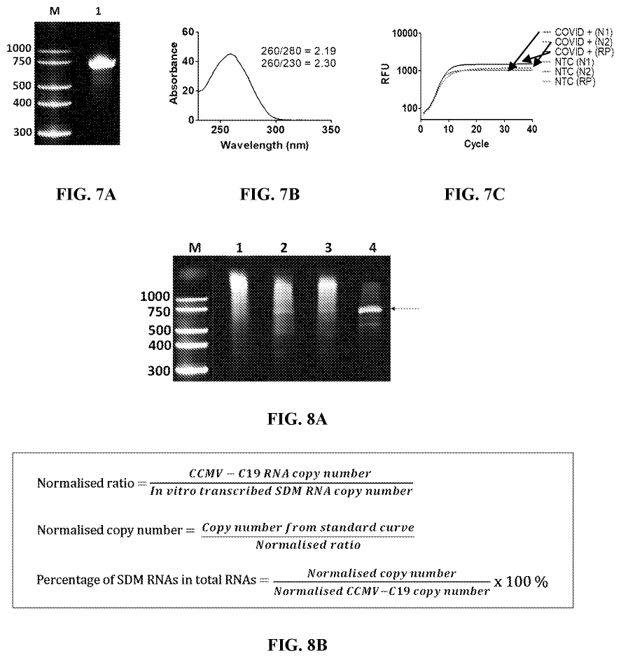

Several safety measures were built into the synthesis of these positive controls. SDM+T7_{P/T} was cloned out-of-frame with the open reading frame to avoid protein translation of truncated SARS-CoV-2 N1 and N2 gene segments. The ribosome binding site (RBS) upstream of SDM was removed for the same reasons. Furthermore, the presence of RBS upstream of target RNA has been shown to reduce packaging efficiency of RNAs into VLPs due to competitive binding of ribosomes versus Qβ CPs to the target RNA (Fang et al., 2018). Therefore, for the one- and two-plasmid systems, the RBS was removed upstream of the SDM, but RBS was retained upstream of the Qβ CPs to enable protein translation. In vitro transcription of SDM RNAs yielded approximately 150 pg of SDM RNAs per reaction; denaturing urea polyacrylamide gel electrophoresis of the transcribed SDM RNA confirmed the ˜622 nt RNA product (FIG. 7A). Lastly, RT-qPCR assays were used to confirm that the SDM provided a template for the CDC primer/probe sets (FIG. 7C).

Production of VLP-Based SARS-CoV-2 Positive Controls

In vivo encapsidation of SDM RNAs in Qβ VLPs was achieved by co-expression of Qβ CPs and SDM RNAs in E. coli using the aforementioned one-plasmid system and two-plasmid system to produce Qβ 1P-C19 VLPs and Qβ 2P-C19 VLPs, respectively. Production of Qβ 2P-C19 VLPs using the two-plasmid system was performed by transforming plasmid pCDFDuet-Qβ and plasmid Qβ 2P-C19 into the same bacterial cells for co-expression of Qβ CPs and SDM RNAs. The one-plasmid system Qβ 1P-C19 was also developed from a single plasmid using the pCDFDuet™-1 vector.

For CCMV-C19, native CCMV was obtained from infected black-eyed pea No. 5 plants and disassembled to obtain purified CPs. The purified CPs were then reassembled with in vitro transcribed SDM RNAs (FIG. 1B). Disassembly and reassembly are achieved through careful adjustment of the buffer conditions (see Materials and Methods section). The negatively charged SDM RNAs interact with the CCMV CP, specifically with the highly positively-charged, arginine-rich binding domains at the N-terminus (amino acids 9-19; sequence: TRAQRRAAARK. SEQ ID NO: 3) (see, e.g., Pretto et al., Bioconjug Chem. 2019 Dec. 18; 30(12):3069-3077; and Annamalai et al., J Virol. 2005 March; 79(6):3277-88); thus reconstitution of the chimeric CCMV VLP with the packaged SDM is achieved through electrostatic interactions.

The following SARS-CoV-2 positive controls were generated: Qβ 1P-C19, Qβ 2P-C19, and CCMV-C19. Expression of the in vivo reconstituted Qβ samples yielded 100 mg/L per batch of culture for Qβ 1P-C19 and Qβ 2P-C19. For CCMV, 100 g of CCMV-infected leaves yielded approximately 40 mg of CCMV nanoparticles. About 20 mg of CCMV VLPs was obtained after disassembly and about 10 mg of SDM encapsidated CCMV-C19 was obtained.

Characterization of the VLP-based SARS-CoV-2 RT-PCR positive controls revealed production of intact, pure, and monodisperse chimeric VLPs (FIG. 2). Separation of intact VLPs on native agarose gels indicated successful RNA encapsidation, as the RNA and protein co-migrate, yielding overlapping and discrete bands when stained with GELRED™ (RNA stain) and Coomassie blue (protein stain). The VLPs have an overall negative charge and therefore migrate toward the anode. See, e.g., Garmann et al., J Virol. 2014 September; 88(18):10472-9; and Doucet et al., PLoS One. 2017 Aug. 10; 12(8):e0181844. The size of the VLPs was determined by dynamic light scattering (DLS) and transmission electron microscopy (TEM). DLS of Qβ 1P-C19 and Qβ 2P-C19 revealed the presence of monodisperse nanoparticles with an average diameter of ˜32 nm (PD ˜0.12-0.13). For CCMV-C19, DLS also revealed monodisperse nanoparticles with a hydrodynamic radius of ˜33 nm (polydispersity index (PDI)=0.128). In all cases, broken particles, free CPs, or aggregates were not detected. These findings are in agreement with TEM imaging showing intact and uniform VLPs with Qβ measuring ˜26-27 nm and CCMV measuring ˜29 nm (FIG. 2); the differences between DLS and TEM—specifically, the increased size as measured by DLS is explained by the hydration status of the sample; whereas ‘dry’ samples are imaged in TEM, DLS reports the hydrodynamic particle size of VLPs in solution. See, e.g., Maguire et al., Sci Technol Adv Mater. 2018 Oct. 18; 19(1):732-745. Size-exclusion chromatography (SEC) for all positive controls showed typical elution profiles for VLPs, where nucleic acid (260 nm) and protein (280 nm) were eluted concurrently at around 12 mL from a Superose 6 column. See, e.g., Doucet et al.; and Cai et al., Adv Funct Mater. 2020 Apr. 14; 30(15):1908743. Also, the SEC results confirmed the structural integrity of particles, showing no free RNAs or CPs in the VLP preparations. Lastly, the particle properties of the chimeric VLPs packaging the SDM matched data of Qβ VLPs devoid of the SDM (FIG. 5) as well as native CCMV particles (FIG. 6).

Validation of the VLP-Based SARS-CoV-2 Positive Controls

As a benchmark, the encapsidated RNAs within the positive controls were extracted using QIAGEN QIAamp viral RNA mini kit; the procedure followed CDC recommendations. One difference to note between SARS-CoV-2 and the positive controls as disclosed herein is that SARS-CoV-2 is an enveloped virus (Abu-Farha et al., Int J Mol Sci. 2020 May 17; 21(10):3544), but the positive controls as disclosed herein based on Qβ and CCMV are non-enveloped viruses. Nevertheless, the extraction protocol is highly suitable for RNA extraction from viruses and the lysis buffer (buffer AVL) used in this study has been optimized by the manufacturer to isolate RNA from a wide variety of viruses, enveloped and non-enveloped. Moreover, the QIAGEN QIAamp® viral RNA mini kit (RNA isolation kit used in this study) is recommended by CDC and has previously been reported to isolate RNA from both enveloped and non-enveloped viruses. See, e.g., Lewandowska et al., Microbiome. 2017 Aug. 8; 5(1):94. Erratum in: Microbiome. 2017 Oct. 12; 5(1):137; and Zhang et al., BMC Genomics. 2018 Oct. 25; 19(1):773. For these reasons, the structural difference between SARS-CoV-2 and the VLP control probes should not affect the results of the diagnostic assay. RNAs were extracted and then eluted in RNase-free water instead of the CDC-recommended AVE buffer for quantification and purity check. Sodium azide in the AVE buffer interferes with absorbance readings between 220 and 280 nm and was thus avoided for the quality control studies. Total nucleic acids extracted include carrier RNAs (poly A, as per manufacturer's instructions), the target SDM RNAs, and, in the case of QP, also random host RNAs that are packed in vivo. Here, the amount of carrier RNAs was assumed constant for each sample. When Qβ 1P-C19 and Qβ 2P-C19 were compared to Qβ VLPs, Qβ 1P-C19 and Qβ 2P-C19 packaged ˜30% more total nucleic acids compared to Qβ VLPs devoid of the SDM (Table 3). The higher nucleic acid content for the designer VLPs Qβ 1P-C19 and QP 2P-C19 can be attributed to the in vivo transcribed SDM RNAs with the Qβ hairpin loop. While Qβ can package RNA non-selectively based on electrostatic interactions of negatively charged cellular RNAs to the positively charged EF-loop on Qβ coat proteins (Cui et al., Proc Natl Acad Sci USA. 2017 Oct. 31; 114(44):11697-11702), the 29-nt Qβ hairpin loop added to the SDM confers high affinity for Qβ CP (Witherell et al.; and Ashcroft et al. J Nanosci Nanotechnol. 2005 December; 5(12):2034-41) and therefore enhances packaging and encapsidation efficiency of the SDM vs. random host RNA. While there was no significant difference in the mean of total nucleic acids extracted from Qβ 1P-C19 and Qβ 2P-C19, Qβ 2P-C19 showed higher batch-to-batch variability with the amount of total nucleic acid extracted from Qβ 2P-C19 ranging from 230 ng/μL to 360 ng/μL. Without wishing to be bound by the theory, this indicates that the two-plasmid system may be less reproducible and that the one-plasmid system would be the preferred system based on the higher quality control and assurance provided. Extracted RNA was analyzed by denaturing urea polyacrylamide gel electrophoresis and staining of the nucleic acids and imaging under UV light revealed presence of the SDM (622 nt). In addition, background RNAs were observed, which can be attributed to the packaging of host RNA as described above (FIG. 8A, Lane 1-3). CCMV-C19 showed the lowest amount of total nucleic acids encapsidated, or about 50% of the total RNA extracted from Qβ positive controls. However, in stark contrast to QP, where the SDM only makes a fraction of the total RNA packaged (Table 3), for CCMV-C19, 100% of the RNA encapsidated into CCMV-C19 is the target SDM RNA (FIG. 8A)—this is an advantage of the in vitro assembly system. Based on BCA assay and RiboGreen nucleic acid assay (Table 6), about 3 SDM RNA molecules were packaged per CCMV-C19; and this is consistent with previous reports, e.g. reporting that 4 copies of 500-nt long RNAs could be encapsidated per CCMV. See, e.g., Cadena-Nava et al., J Virol. 2012 March; 86(6):3318-26. Epub 2011 Dec. 28. Erratum in: J Virol. 2019 Feb. 5; 93(4). In contrast, one SDM RNA molecule was encapsidated in every 17^{th} Qβ 1P-C19 particle and 30^{th} Qβ 2P-C19 particle; the inefficiency in packaging can be explained by competition of the SDM target cargo with host E. coli RNAs.

TABLE 4
Stability study: Analysis of RNAs from one-
month old VLP-based SARS-CoV-2 positive controls.
Total nucleic acids include carrier RNA.

| | Qβ 1P-C19 | Qβ 2P-C19 | CCMV-C19 |
|---|---|---|---|
| Total nucleic acids | 223 | 169 | 106 |
| (ng/μg VLP) | | | |
| 260/280 | 2.58 | 2.54 | 2.89 |
| 260/230 | 2.74 | 5.56 | 3.19 |

TABLE 5
Sequences of ddPCR primers and probes used in
clinical settings.

| | SEQ | |
|---|---|---|
| Primer/ | ID | |
| probe | NO: | Sequence (5′-3′) |
| N1-F | 4 | GAC CCC AAA ATC AGC GAA AT |
| N1-R | 5 | TCT GGT TAC TGC CAG TTG AAT CTG |
| N1-P | 6 | FAM/ZEN - ACC CCG CAT TAC GTT TGG |
| | | TGG ACC - IBFQ |
| N2-F | 7 | TTA CAA ACA TTG GCC GCA AA |
| N2-R | 8 | GCG CGA CAT TCC GAA GAA |
| N2-P | 9 | FAM/ZEN - ACA ATT TGC CCC CAG CGC |
| | | TTC AG - IBFQ |
| RP-F | 10 | GATTTGGACCTGCGAGCG |
| RP-R | 11 | GCGGCTGTCTCCACAAGT |
| RP-P | 12 | HEX/ZEN - CTGACCTGAAGGCTCT- IBFQ |

*F refers to forward; R refers to reverse; P refers to probe

TABLE 6
Calculation of SDM RNA molecule encapsidated in VLPs.

| | Qβ 1P-C19 | Qβ 2P-C19 | CCMV-C19 |
|---|---|---|---|
| Protein concentration | 2.60 × 10⁻⁵ | 2.94 × 10⁻⁴ | 2.66 × 10⁻⁵ |
| (μg*mol/g*mL) | | | |
| Total RNA concentration | 7.42 | 51.63 | 16.71 |
| (μg/mL) | | | |
| Percentage of SDM RNA | 3.98 | 3.80 | 100 |
| in total RNA (%) | | | |
| SDM RNA concentration | 1.48 × 10⁻⁶ | 9.86 × 10⁻⁶ | 8.40 × 10⁻⁵ |
| (μg*mol/g*mL) | | | |
| Number of SDM | 0.06 | 0.03 | 3.15 |
| RNA/particle | | | |

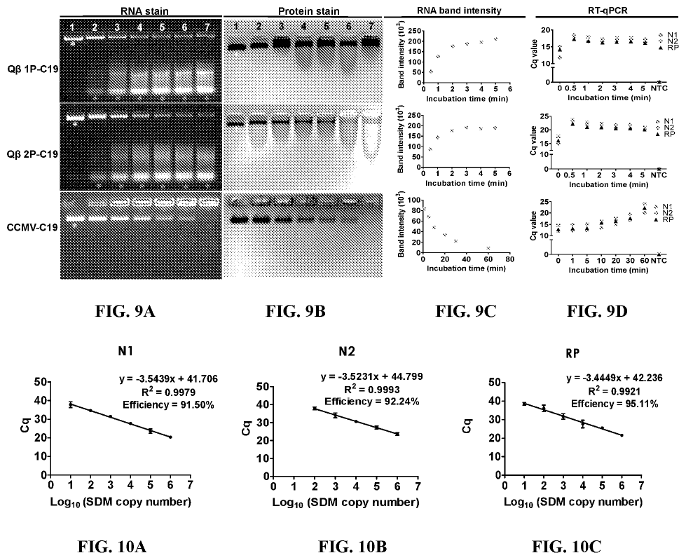

The encapsulation of SDM into VLPs was further validated by investigating the release kinetics of SDM from the Qβ and CCMV-based probes. VLPs (Qβ 1P-C19, Qβ 2P-C19, CCMV-C19) were heated at 75° C. and sampled; first samples were analyzed by agarose gel electrophoresis; RNA bands were then extracted and subjected to RT-qPCR analysis; a time course study was performed and released RNA samples over a 5-min time course (QP 1P-C19, Qβ 2P-C19) and 60 min time course (CCMV-C19). Stable encapsulation is confirmed at t=0 min with the protein and RNA stain co-localizing after electrophoretic separation (FIG. 9). Upon heating of the samples, released RNA was also apparent and the intensity increased with increasing incubation time. The heating also reduced the mobility of the coat proteins, which further suggests disassembly and RNA release. See, e.g., Hovlid et al., ACS Nano. 2014 Aug. 26; 8(8):8003-14; and de Ruiter et al., J Control Release. 2019 Aug. 10; 307:342-354.

To confirm that SDMs were indeed encapsidated into the VLPs, RNAs from time point 0 (intact particles) were extracted from gels and validated by RT-qPCR. Presence of N1, N2, and RP fragments was confirmed (FIG. 9; RT-qPCR panel). RT-qPCR analysis was also carried out on RNA released from the Qβ 1P-C19 and Qβ 2P-C19 VLPs upon heating at 75° C. and presence of SDM RNAs and its N1, N2, and RP was confirmed. The amount of SDM RNAs quantified by RT-qPCR matched the RNA band intensity measured. It was interesting to note that the Qβ particle appeared to remain stable while releasing its RNA content upon exposure to high temperature. This may be explained by the porous nature of the Qβ capsid combined with its high thermal stability. See, e.g., Fiedler et al., Biomacromolecules. 2012 Aug. 13; 13(8):2339-48.

Reduced RNA content led to slower mobility of the Qβ particles, and this is consistent with previous observations (Hovlid et al.). Longer incubation at high temperature was required to achieve effective release of RNA from CCMV. While Qβ-based positive controls released nearly all of their RNA content within 5 min at 75° C., CCMV required exposure for at least 30 min at 75° C. to observe significant RNA release. CCMV did not appear to remain stable under these conditions. The decrease in protein content with increasing incubation time indicates that the CCMV particle disassembled or denatured. This process would release the RNA; staining indicates that RNA, at least in part, aggregates with the CP, as indicated by RNA and protein stain in the pockets of the gel. Nevertheless, also, for the CCMV-C19 positive controls, RT-qPCR confirmed the presence of N1, N2, and RP.

Amplification efficiency is one of the most important factors in qPCR. An ideal amplification efficiency of 100% corresponds to exponential doubling of the PCR product during every cycle to give an amplification factor of 2. See, e.g., Gibson et al., Genome Res. 1996 October; 6(10):995-1001. The slope of the standard curve is used to tabulate the amplification efficiency, with the equation: E=(10^{-1/slope}−1)×100%. See, e.g., Wittwer, C. Rapid Cycle Real-Time PCR: Methods and Applications. In Rapid Cycle Real-Time PCR; Springer, 2001; pp 1-8. Amplification efficiency of the CDC primer/probe set has been validated with synthetic SARS-CoV-2 RNA transcripts as well as clinical samples. See, e.g., Vogels et al.; and Jung et al., ACS Infect Dis. 2020 Sep. 11; 6(9):2513-2523. N1 and N2 primer sets have reported amplification efficiencies of more than 90%, which validates that the primers are optimized for SARS-CoV-2 RNA binding. Here, the amplification efficiency of the CDC primers was confirmed on the SDM RNA as disclosed herein. All three regions (N1, N2, RP) showed amplification efficiency above 90%, with correlation coefficient (R²) >0.99 (FIG. 10). N1 and RP primer/probe sets demonstrated sensitivity higher than that of the N2 positive controls at 10¹ copies/μL for Cq<40; Cq is defined as quantification cycle in RT-qPCR. For the N2 primer/probe set, the detection limit was 10² copies/μL. This result aligned with a previous report when using real patient samples (Vogels et al.), indicating that the SARS-CoV-2 positive controls as disclosed herein are able to function as a SARS-CoV-2 mimic for RT-qPCR detection.

Using the RT-qPCR method, it was determined that the SDM RNA copy number per nanogram of total nucleic acids extracted (after normalization of the Cq value, see FIG. 8B).

The SDM RNA copy number was tabulated from mean copy number of N1, N2, and RP based on the standard curves shown in FIG. 10. Proper baseline settings are critical for accuracy. See, e.g., Ruijter et al., Nucleic Acids Res. 2009 April; 37(6):e45. Therefore, to standardize the baseline setting in RT-qPCR, the quantification cycle (Cq) value of CCMV-C19 was first normalized to in vitro transcribed SDM RNA by assuming that total RNAs extracted from CCMV-C19 contain only SDM RNA. The normalized ratio is then applied to Cq values of Qβ 1P-C19 and Qβ 2P-C19, as mentioned in FIG. 8B, to obtain the percentage of SDM RNAs in total RNA extracted. CCMV-C19 has the highest SDM RNA copy number, with >10⁹ copies per nanogram of total nucleic acids (Table 3). This is as expected, because CCMV-C19 was assembled in vitro using only SDM target RNAs. Qβ 1P-C19 and Qβ 2P-C19 have similar copy number of SDM RNA, or around 10⁸ for every nanogram of total nucleic acids (Table 3). The lower copy number is due to encapsidation of cellular E. coli RNAs, which has been shown in FIG. 8A. All CDC primer/probe sets demonstrated no binding to random cellular E. coli RNAs that encapsidated in Qβ.

There was no significant difference in SDM RNA copy number between Qβ 1P-C19 and Qβ 2P-C19 (unpaired t-test, p >0.05), suggesting that the one-plasmid system with indirect upstream RBS has a packaging efficiency similar to that of the two-plasmid system (though the two-plasmid system resulted in greater batch-to-batch variability, as discussed above). The SDM RNAs made up about 4% of the total RNAs encapsidated in QP. Despite the target RNAs only being a fraction of the total RNAs extracted, the copy number of encapsidated SDM RNAs is still sufficiently high to function as a positive control. Early infection yields about 6.76×10⁵ copies per whole swab (Wolfel et al.). At least 10⁸ copies of SDM RNAs were obtained from every microgram of VLP (similarly to every 5 ng of total nucleic acids extracted). Compared to other VLPs proposed as SARS-CoV-2 positive controls, the Qβ VLPs offer higher production efficiency, yielding VLPs at packing with about 100-fold mores copies of RNA compared to, for example, MS2 bacteriophage (Yao et al.).

Validation of the VLP-Based SARS-CoV-2 Positive Controls in the Clinical Setting

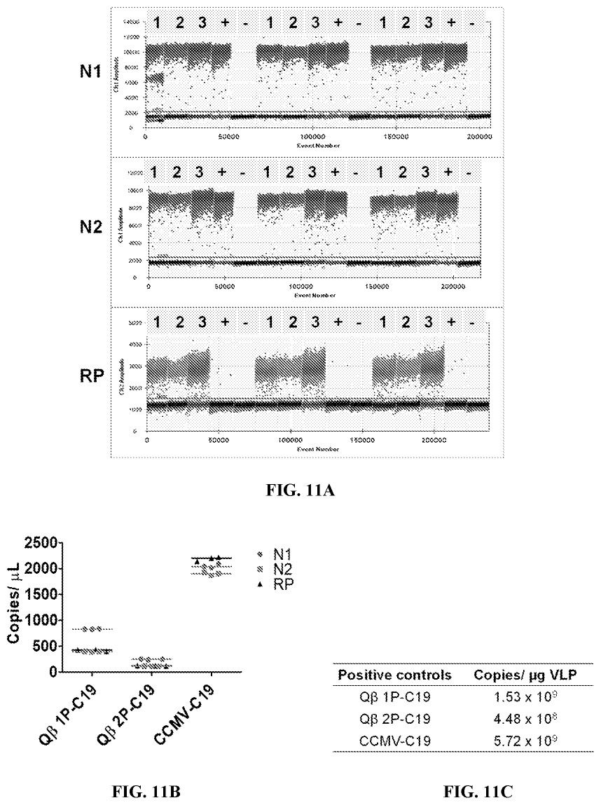

In-house VLP-based SARS-CoV-2 positive controls were validated in clinical settings by comparing performance alongside with clinical samples using a droplet digital PCR system (ddPCR). Clinical testing revealed that the VLP-based SARS-CoV-2 positive controls yielded amplitude signals between 2000 to 10,000 for all the three regions (N1, N2, RP) (FIG. 11A). The 1-D amplitude plot confirmed that the in-house VLP-based SARS-CoV-2 positive controls present a functional SARS-CoV-2 RNA mimic and enable detection using the CDC primer/probe set (FIG. 11A). All three detection regions recommended by CDC (N1, N2, RP) in all three SARS-CoV-2 positive controls, Qβ 1P-C19, Qβ 2P-C19, and CCMV-C19, were successfully detected. Importantly, it was possible to process the positive controls alongside the clinical samples throughout every step, starting from RNA extraction though amplification, providing seamless full-process control.

Consistent with the above-described findings, CCMV-C19 probes were the most sensitive, as reflected by the highest copy numbers detected (>2,000 copies/μL, FIG. 11B); this is consistent with CCMV packaging the highest copy number of SDM. Fewer copies of SDM were packaged in Qβ 1P-C19 and Qβ 2P-C19 (due to encapsidation of random host RNAs) and this resulted in lower SDM copies detected (<1,000 copies/μL and <500 copies/μL, respectively, FIG. 11B). Overall, the data were aligned with the results obtained from RT-qPCR (Table 7).

When comparing the performance of Qβ 1P-C19 vs. Qβ 2P-C19, it was interesting to note the differences comparing ddPCR vs. RT-qPCR: while RT-qPCR showed no apparent differences in copy number of SDM (Table 3), ddPCR indicated higher SDM copy for QP 1P-C19 vs. Qβ 2P-C19, with 1.53×10⁹ SDM/pg VLP vs. 4.48×10⁸ SDM/pg VLP (FIG. 11C). The introduction of PCR inhibitors at the RNA extraction stage may account for the reduced sensitivity of the RT-qPCR method. See, e.g., Schrader et al., J Appl Microbiol. 2012 November; 113(5):1014-26. Previous studies have shown that ddPCR method exhibits higher tolerance to PCR inhibitors compared to conventional RT-qPCR and this is explained by reaction partitioning. See, e.g., Dingle et al., Clin Chem. 2013 November; 59(11):1670-2; and Sidstedt et al., Anal Chem. 2017 Feb. 7; 89(3):1642-1649. Also, droplet-based reaction partitioning in ddPCR leads to increased variance in SDM copy number when comparing the positive controls. Regardless of the differences in performance observed, each of the proposed SARS-CoV-2 positive controls—Qβ 1P-C19, Qβ 2P-C19, and CCMV-C19—provided robust positive controls with at least 10⁸ SDM copies from every microgram of VLP (FIG. 11C).

Stability of the VLP-Based SARS-CoV-2 Positive Controls

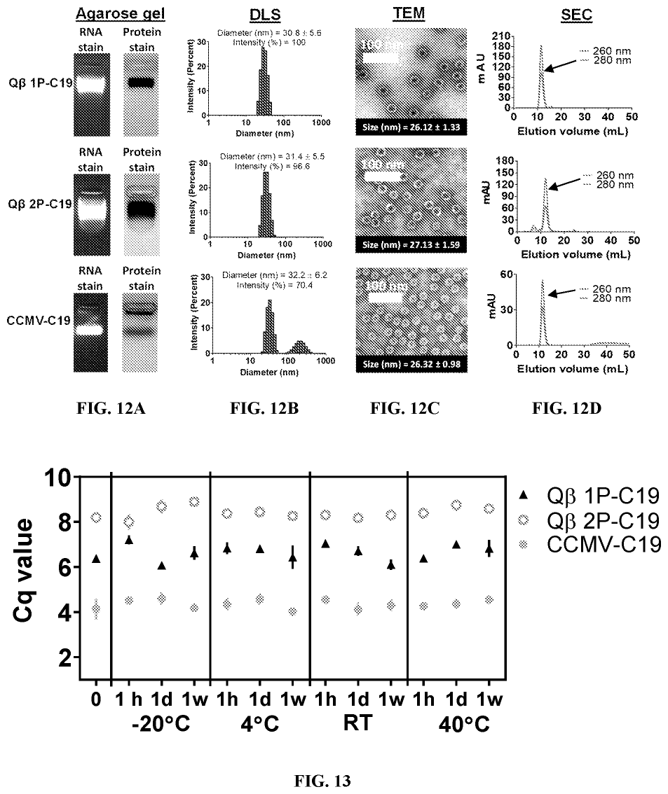

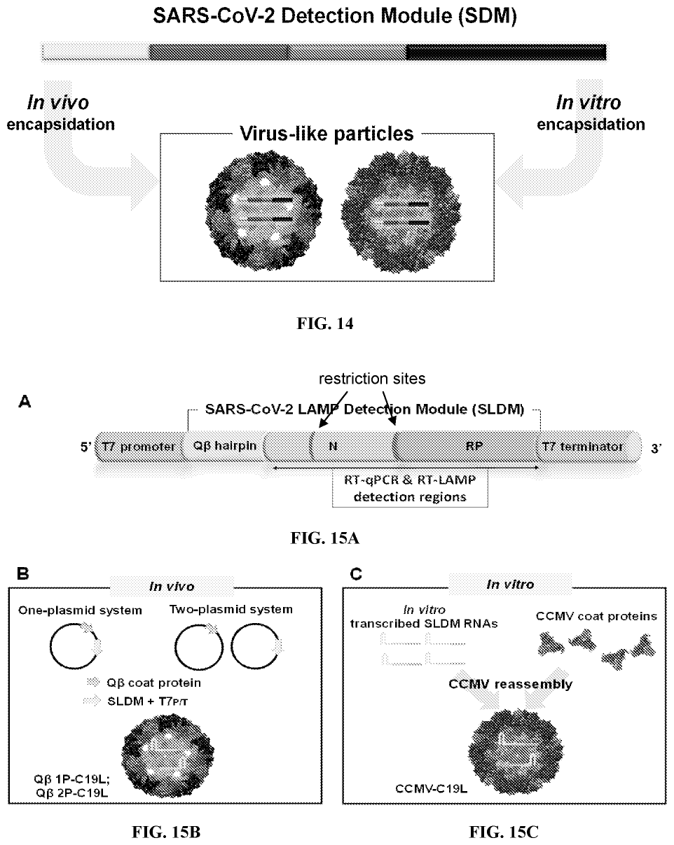

Most of the commercially available positive controls require cold-chain distribution in order to maintain product quality. However, the pipeline of cold-chain distribution starting from packaging to logistics increases the cost of the product and limits their widespread distribution. Cold-chain infrastructure is not available in low-resource settings and is especially problematic in countries with tropical climates. Bacteriophages and plant viruses evolved as highly stable capsids that retain their structural properties under various environmental conditions, making them suitable candidates for development of positive controls that do not require a cold-chain infrastructure. To investigate the stability of the in-house SARS-CoV-2 positive controls under conditions that simulate shipping conditions, samples were left at room temperature (approximately 20-25° C.) for a month. Structural integrity and SDM functionality were then assessed using agarose gel electrophoresis, DLS, TEM, and SEC, as well as RT-qPCR. Qβ 1P-C19 and Qβ 2P-C19 exposed to these conditions remained intact. There was no aggregation and there were no free CPs to indicate disassembly of the Qβ positive controls, and the particle characteristics matched those obtained of freshly prepared particles (FIG. 12 vs. FIG. 2). The excellent stability of Qβ can be attributed to the two cysteine residues on the Qβ coat protein forming inter-subunit disulfide bonds (Ashcroft et al.).

The CCMV-C19 positive control was found to be less stable, however still yielding a recovery of ˜70% intact CCMV-C19 positive control after 1-month storage at ambient conditions (FIG. 12, DLS panel). The CCMV assembly is stabilized by electrostatic and hydrophobic interactions. See., e.g., Speir et al., Structure 1995, 3, 63-78; and Tama et al., J. Mol. Biol. 2002, 318, 733-747. However, the lack of disulfide bonds in CCMV reduces longitudinal particle stability. See, e.g., Mateu. Protein Eng Des Sel 2011, 24, 53-63. Data are consistent and indicate partial disassembly leading to some degree of aggregation: native gel electrophoresis revealed intact particles with RNA and CPs having matched electrophoretic mobility; however a second protein band appeared near the well, indicating aggregation of CPs and RNA (FIG. 12, Lane 2). While the RNA stain was less obvious, this pattern may indicate a mixture of disassembled CPs associated and/or aggregated with RNA via their positively-charged N-terminal tails. See, e.g., Culver et al., Virology 2015, 479, 200-212. VLP aggregation was not apparent from TEM imaging, therefore the agarose gel electrophoretic pattern as well as the observed aggregates in the DLS measurement was attributed to partial disassembly of CPs and their aggregation as CP-RNA complexes. Indeed, SEC of CCMV-C19 indicates a second broadened elution peak at 35-50 mLs. For application in the clinic, it may be desired to stabilize the CCMV capsid through introduction of covalent bonds between CPs; this concept was recently demonstrated using a homobifunctional cross-linker 3, 3′-dithiobis (sulfosuccinimidylpropionate) (DTSSP) to form intraparticle cross-links between lysine side chains of adjacent CPs (Pretto et al.).

Next, and most importantly, SDM stability was assayed. While no structural changes where observed for the Qβ 1P-C19 and Qβ 2P-C19 positive controls, RT-qPCR data indicate that the SDM copy number was reduced by 30-35% (Table 7). This observation is aligned with the reduction of total nucleic acid extracted from one-month-old positive controls (Table 4). Without wishing to be bound by the theory, the reduction of RNAs is due to hydrolysis contributed by water molecules in buffered solution providing hydroxyl or hydronium ions for proton transfer. See, e.g., Seyhan and Burke. Rna 2000, 6, 189-198. The Qβ capsid is a porous structure and has pores measuring 1.3-1.4 nm at its 3-fold axis as well as 0.7 nm-sized pores at its 5-fold axis. These pores facilitate diffusion of water (causing RNA hydrolysis), ions, as well as degraded RNAs (leading to loss of RNA content) (Golmohammadi et al.; and Hovlid et al.). A possible strategy to stabilize the Qβ positive control for future applications may be to plug the pores through appropriate chemistry or through freeze-drying in appropriate excipients. See, e.g., Lang et al., Drug Dev. Ind. Pharm. 2009, 35, 83-97.

Lastly, an expected reduction in SDM copy number was also apparent for CCMV-C19. A 30% reduction of SDM was estimated from the dissociation observed (FIG. 12), and this is consistent with reduction of total nucleic acids (Table 4) after the 1-month storage period.

TABLE 7
Normalized copy number of SDM per 5 ng of total
nucleic acids after 1-month storage of the
VLP-based SARS-COV-2 positive controls.

| | | Percentage of SDM | Percentage of |
|---|---|---|---|
| | Normalized copy | RNAs in total RNAs | reduction in copy |
| | number (10\hat{ }) | (%) | number (%) |
| Qβ 1P-C19 | 8.63 ± 0.17 | 2.82 | 29.21 |
| Qβ 2P-C19 | 8.57 ± 0.25 | 2.45 | 35.43 |
| CCMV-C19 | 10.18 ± 0.42 | 100.00 | n/a |

The VLP probe stability was further challenged with respect to time and temperature by incubating the three VLPs (Qβ 1P-C19, Qβ 2P-C19, and CCMV-C19) at four different temperatures (−20° C., 4° C., room temperature: 20° C.-25° C., 40° C.) for 1 h, 1 day, and 1 week, respectively (FIG. 13). These conditions mimic most of the shipping and storage conditions. Data indicate no apparent reduction in Cq value which correlates to SDM RNA copy number under any of the environmental conditions tested. CCMV-C19 encapsidated the highest amount of SDM RNAs by giving the lowest overall Cq values compared to its QP companions. Qβ 2P-C19 has the highest overall Cq values due to encapsidation of lowest amount of SDM RNAs as mentioned previously. Cq values fluctuated within ±0.55, ±0.45, and ±0.29 cycles for Qβ 1P-C19, Qβ 2P-C19, and CCMV-C19, respectively. It should be noted that fixed volume instead of fixed amount of RNAs was applied in the RT-qPCR assay, therefore variation in Cq values correlates to the amount of target SDM. CCMV-C19 showed the most consistent Cq values with ˜16% variation in SDM RNA copy number over the various environmental conditions and time course. This may be attributed to the fact that CCMV encapsidates only the synthetic SDM RNAs; in contrast QP-based probes also harbor a significant amount of ‘junk’ RNAs that may impact the quality of the RT-qPCR. Moreover, the increased variation in Cq values for the Qβ 1P-C19, and Qβ 2P-C19, with ˜25% fluctuation, may also be explained by the porous nature of the capsid which may be more prone to RNA hydrolysis (Seyhan and Burke). Nevertheless, overall stability of the VLP constructs is maintained and demonstrated over the 1-week time course at various temperatures; even incubation at 40° C. for one week did not significantly impact the SDM packaged in the VLP probes; therefore the biomimetic control probes as disclosed herein can be suitable for use also in low-resource settings.

### Conclusions

In conclusion, both plant virus-derived CCMV and bacteriophage-derived Qβ VLPs offer promising platforms for the encapsidation of RNA modules and application as stable, widely applicable positive controls for RT-qPCR or ddPCR detection of infectious agents, such as SARS-CoV-2. In vitro reconstitution protocols and in vivo expression systems was developed, yielding CCMV- and QP-based nanoparticles encapsidating SARS-CoV-2 detection modules that are compatible with the CDC primer/probe sets. Because the RNA is stabilized inside the VLP particle, the positive control mimics the conditions encountered by the RNA template of SARS-CoV-2 within clinical or environmental samples. The increased stability also enables for these positive controls to be applied as full-process controls, as demonstrated in the clinical assays performed. Both, the CCMV and Qβ platforms are massively scalable through manufacture via plant molecular farming and bacterial fermentation. The in vivo expression of Qβ VLPs offers the advantage of fewer processing steps compared to in vitro dis-assembly and reassembly used to obtain the chimeric CCMV VLPs. The latter method does offer control of target molecule encapsidation: while the payload of CCMV is solely SDM (˜10⁹ SDM copies/μg VLP), only a fraction of the QP payload is SDM (˜10⁸ SDM copies/μg VLP), with a large portion of the cargo being non-target host RNAs. Nevertheless, both VLP systems were robust in clinical assays. Importantly, the developed positive controls are safe and avoid the risks of using RNA extracted from infected patients. The positive controls demonstrated considerable stability over 1-month at ambient conditions; further, the probes offered excellent stability at temperatures as high as 40° C. over one-week (longer time periods were not tested). Together, these attributes made the handling of SARS-CoV-2 positive controls safe and accessible for clinical personnel in a wide range of settings; given the ease of manufacture and stability conferred over a range of environmental setting, the proposed designs may aid diagnostic testing not just in testing facilities, but also at the point of entry (i.e. at airports or border crossings) and in low-resource areas. The potential to make these SARS-CoV-2 positive controls widely available at ambient conditions could help to alleviate some of the disparities in testing that are contributing to the increased COVID-19-related deaths in underserved populations in the US and across the world.

### Materials and Methods

Construction of Qβ 1P-C19 and Qβ 2P-C19 Plasmids

Qβ 1P-C19: SARS-CoV-2 detection module (SDM) was synthesized and cloned into pCDFDuet-Qβ between restriction site NotI and NdeI to generate Qβ 1P-C19 plasmid (GenScript, FIG. 3). The gene was cloned out-of-frame with the open reading frame (for safety reason; the placement out of open reading frame avoids translation) and placed downstream of Qβ coat protein gene.

Qβ2P-C19: SARS-CoV-2 detection module (SDM) gene from plasmid Qβ 1P-C19 was subcloned into plasmid pET-28a(+) by amplifying with Qβ 2P-C19 Forward primer (5′-GAA GAT CTT AAT ACG ACT CAC TAT AGG G-3′, SEQ ID NO: 13) and Qβ 2P-C19 Reverse primer (5′-TTT TCC TTT TGC GGC CGC CAA AAA ACC CCT CAA GAC CCG TTT AGA G-3′, SEQ ID NO: 14) using NEB Q5© High Fidelity 2× Master Mix. The gene was cloned between restriction site BgIII and NotI in pET-28a(+), devoid of ribosome binding site at upstream to generate plasmid Qβ 2P-C19. This plasmid was used to express SDM RNAs in vivo. Plasmid pCDFDuet-Qβ was co-transformed with plasmid Qβ 2P-C19 to express Qβ coat proteins for in vivo reconstitution of SDM RNAs. The clone was subjected to DNA Sanger sequencing (Eurofins Genomics) to confirm the insertion.

Production of Qβ 1P-C19 and Qβ 2P-C19 VLPs

Plasmids were transformed into BL21 (DE3) competent E. coli cells (New England BIOLABS®) and plated out on antibiotic containing plate. Selection of Qβ 1P-C19 transformants was based on streptomycin resistance (100 μg/mL); while Qβ 2P-C19 transformants was based on streptomycin resistance (100 μg/mL) and kanamycin (50 μg/mL). The E. coli was inoculated in Luria-Bertani (LB) media supplemented with antibiotic and incubated overnight at 37° C. with shaking at 250 rpm. For VLP expression, the overnight culture was diluted 1:100 in Thermo Fisher Scientific's MAGICMEDIA™ E. coli Expression Medium and incubated overnight at 30° C. with shaking at 300 rpm. The culture was centrifuged at 9800×g for 10 min at 4° C. The cell pellet was resuspended with 0.1 volume of 1× phosphate buffered saline (PBS), pH 7.4 supplemented with 0.2 mg/mL lysozyme and sonicated for 10 min with 5 sec on/off cycle. The lysate was then centrifuged at 9800×g for 15 min to collect the supernatant. Crude VLPs in supernatant were pelleted down with 10% (w/v) PEG 8000 and 0.2 M NaCl followed by resuspension in 1×PBS (pH 7.4). Crude VLPs were treated with 0.7 volumes of 1:1 chloroform: butanol mixture and the upper aqueous layer was purified on 10-40% (w/v) sucrose gradient by centrifugation at 133,000×g for 3 h, 4° C. The band with VLPs was collected and centrifuged at 210,000 g for 3 h, 4° C. The final pellet was resuspended in 1×PBS (pH 7.4) and the VLPs were stored at −20° C. VLP concentration was measured with PIERCE™ BCA protein assay kit.

In Vitro Transcription of SARS-CoV-2 Detection Module

Plasmid Qβ 1P-C19 was digested with NotI and NdeI to obtain the linearized SDM. In vitro RNA transcription of SDM was performed with Thermo Fisher Scientific's MEGASCRIPT™ T7 Transcription kit and purified with INVITROGEN™'s MEGACLEAR™ Transcription Clean-Up kit. Purity and concentration of transcribed RNAs were validated with THERMO SCIENTIFIC™ Nanodrop 2000/2000c at ratio 260/280 and 260/230. RNA concentration is determined from A260 with a reading of 1.0 being equivalent to about 40 ng/μL of RNA. Pure RNA should yield around 2 or higher for both ratios. In vitro transcribed RNAs were also analyzed using INVITROGEN™ NOVEX™ 6% TBE-Urea gel (cat. no: EC6865BOX).

Production of CCMV-C19 VLPs

Primary leaves of Vigna unguiculate, California black-eyed peas No. 5 were mechanically infected with CCMV after growing for 12 days (these protocols are carried out under USDA-approved P526 permits). Plants were grown for another 12 days before leaves were harvested. CCMV was purified using established procedures. See, e.g., Bancroft. The Self-Assembly of Spherical Plant Viruses. In Advances in virus research; Elsevier, 1970; Vol. 16, pp 99-134. In brief, harvested CCMV infected leaves were homogenized with Preparation Buffer (0.2 M NaOAc, 1 mM EDTA, PH 4.8) and filtered through cheesecloth. Filtrate was later centrifuged at 15,000×g for 15 min. The supernatant was precipitated by 0.02 M NaCl, and 8% (w/v) PEG 8000 and stirred overnight at 4° C. The solution was centrifuged at 15,000×g for 10 min at 4° C. The pellet was resuspended in 20 mL of CCMV Buffer (0.1 M NaOAc, 1 mM EDTA, PH 4.8) followed by centrifugation at 8000×g for 10 min at 4° C. The supernatant was then centrifuged over a 5 mL 20% (w/v) sucrose cushion in water at 148,000×g for 2 h at 4° C. The pellet was resuspended in CCMV Buffer. Purified CCMV was stored as intact virion until further use. Purified coat proteins (CPs) were obtained using established disassembly protocols. See, e.g., Cadena-Nava et al.; and Annamalai and Rao. Virology 2005, 332, 650-658. Disassembled CCMV CPs were reassembled with purified SDM obtained through in vitro transcription from plasmids; the SDM-to-CP ratio was 6:1 as previously described. See, e.g., Cadena-Nava et al. The reconstituted CCMV was stored in CCMV Buffer at −80° C. VLP concentration was measured with PIERCE™ BCA protein assay kit.

Characterization of VLP-Based SARS-CoV-2 Positive Controls

Transmission Electron Microscopy (TEM)

Positive controls (Qβ 1P-C19, Qβ B 2P-C19, CCMV-C19) were diluted to 0.2 mg/mL in Milli-Q water and 4 μL was adsorbed to Formvar/carbon-coated 400 mesh copper grids (Electron Microscopy Science) for 2 min. The grid was washed with 4 μL of water for 1 min followed by adsorption of 4 μL of 2% (w/v) uranyl acetate (Fisher Scientific) for 2 min. Solution was removed from grid by blotting with filter paper. TEM grids were imaged with FEI Tecnai G2 Spirit transmission microscope at 80 kV. The size of particles was analyzed using ImageJ software. 20 particles were randomly selected and their diameters were measured.

Dynamic Light Scattering (DLS)

Qβ 1P-C19 and Qβ 2P-C19 were diluted to 0.5 mg/mL in 1× phosphate buffered saline (PBS, pH 7.4) Qβ 1P-C19 and CCMV-C19 samples were diluted to 0.5 mg/mL using Virus Suspension Buffer (VSB: 50 mM sodium acetate, 8 mM magnesium acetate (pH 4.5)). 60 μL samples were then analyzed with a Malvern Panalytic Zetasizer Nano ZSP.

Agarose Gel Electrophoresis

Qβ1P-C19 and Qβ2P-C19: 10 μg of Qβ 1P-C19 and Qβ 2P-C19 positive controls was loaded onto a 1.2% (w/v) TAE agarose gel. The samples were electrophoresed at 110 V for 40 min.

CCMV-C19: 10 μg of CCMV-C19 in a total volume of 15 μL in VSB was mixed with 3 μL of 100% glycerol before loading into 1% (w/v) agarose gel buffered with virus electrophoresis buffer (0.1 M sodium acetate, 1 mM EDTA, pH 5.5). The samples were electrophoresed at 50 V for 60 min at 4° C. Documentation of gels was performed with ProteinSimple FluorChem R.

Size Exclusion Chromatography (SEC)

200 μL of 0.5 mg/mL of VLP-based SARS-Cov-2 positive controls were analyzed by GE Healthcare Äkta Purifier chromatography system using a Superose 6 column. Samples were analyzed at a flow rate of 0.5 mL/min using 1×PBS for Qβ 1P-C19 and Qβ 2P-C19; VSB for CCMV-C19. Detectors were set at 260 nm (RNA) and 280 nm (protein).

Quantification of Encapsidated SDM RNA Molecules in VLP-Based SARS-Cov-2 Positive Controls

1 μL of VLP was quantified with a PIERCE™ BCA protein assay kit and a Thermo Fisher QUANT-IT™ RIBOGREEN™ RNA Assay Kit according to manufacturer's protocol. Number of encapsidated SDM RNA molecule per VLP was calculated as follows:

$$= \frac{( Concentration of SDM RNA in \frac{ug}{mL} ) \div ( MW of SDM RNA )}{( Concentration of VLP in \frac{ug}{mL} ) \div ( MW of CP \times 180 )}$$

*where MW SDM RNA=199 kDa; MW CCMV CP=21 kDa; and MW Qβ CP=14.3 kDa (MV=molecular weight; kDA=kiloDalton; CP—coat protein).

Extraction of Total RNAs from VLP-Based SARS-Cov-2 Positive Controls

RNA was extracted from VLP-based SARS-Cov-2 positive controls using QIAGEN's QIAamp Viral RNA mini kit according to the manufacturer's protocol. RNA was eluted with 50 μL of nuclease-free water. Purity and concentration of total RNAs was determined by THERMO SCIENTIFIC™ Nanodrop 2000/2000c. Total RNAs were analyzed using Invitrogen's NOVEX™ TBE-Urea gel, 6%. The gels were stained with 2 μL of BioGold GELRED™ Nucleic Acid Stain at 10,000× in 40 mL of water for 20 min and washed off with 40 mL of water for 10 min. Documentation of gels was performed with ProteinSimple FluorChem R.

Quantitative Reverse-Transcription Polymerase Chain Reaction (RT-qPCR): Validation of VLP-Based SARS-CoV-2 Positive Controls

PCR amplification efficiency was performed with a range of SDM RNA transcripts (10⁶ to 10⁰ copy) using 2019-nCoV CDC qPCR Probe Assay from Integrated DNA Technologies (IDT) (cat. no: 10006713) and INVITROGEN™'s SUPERSCRIPT™ III PLATINUM™ One-step RT-qPCR kit (cat. No: 11732020) according to the manufacturer's protocol. Briefly, 2 μL of RNA was used in a 20 μL reaction containing a final concentration of 1× reaction mix, 0.4 μL of SUPERSCRIPT™ III RT/PLATINUM™ Taq Mix, and 1× primer/probes (IDT™). PCR cycling conditions were performed as follows: 50° C. for 15 min, 95° C. for 2 min, followed by 40 cycles of 95° C. for 15 seconds and 60° C. for 30 sec. The RT-qPCR reactions were performed on a BioRad CFX96 TOUCH™ real-time PCR detection system. All samples were run in triplicate. Quantification cycle (Cq) values were tabulated by CFX™ Maestro software.

RNA Release Kinetics from VLP-Based SARS-CoV-2 Positive Controls

SARS-CoV-2 positive control (10 pg) in a total volume of 20 pL was incubated at 75° C. for 0 min, 0.5 min, 1 min, 2 min, 3 min, 4 min, and 5 min for Qβ 1P-C19 and Qβ 2P-C19. CCMV-C19 was incubated at 75° C. for 0, 1, 5, 10, 20, 30, and 60 min. The solution was later analyzed with native agarose gel electrophoresis as mentioned previously and band intensity was analyzed by ImageJ software. The RNA bands were excised with a y scalpel and soaked in 500 μL of 1× TE buffer, Molecular Biology Grade (Promega) supplemented with 40 U RNase inhibitor (APPLIED BIOSYSTEM™). The mixture was incubated at room temperature for 25 min with gentle shaking. The gel was later removed from TE buffer and RNA was extracted using THERMO SCIENTIFIC™ GeneJET Gel Extraction Kit according to manufacturer's protocol with slight modification. Briefly, 1 volume of Binding buffer was added to 1 volume of gel and incubated at 56° C. until dissolved (6-8 min). The solution was transferred to a column and spun for 1 min at 14,550×g. The column was washed twice with 700 μL of Wash buffer by spinning at 14,550×g for 1 min. An additional spin at similar condition was performed to remove residual ethanol. Then, 30 pL of nuclease-free water was added to the center of membrane and incubated at 56° C. for 5 min. Lastly the column was spun at 14,550×g for 1 min to elute the RNA. 1 pL of eluted RNA was quantified with RT-qPCR using N1, N2, and RP primers/probe.

Validation of the VLP-Based SARS-CoV-2 Positive Controls in the Clinical Setting

SARS-CoV-2 positive controls (10 pg) were extracted using QIAGEN's QIAamp Viral RNA mini kit (cat. no: 52904) according to manufacturer's protocol and eluted in 140 μL of AVE buffer. 10 μL of eluted RNA sample was diluted to 10⁻⁶ with RNase-free water. Clinical samples were extracted from a COVID-19 case (positive control patient) and a healthy patient (negative control). Clinical samples were diluted 100 times with RNase-free water. 10 pL of the diluted RNA sample was used to set up singleplex ddPCR in a 20 pL reaction with a Bio-Rad One-step RT-ddPCR Advanced Kit (cat. no: 1864022) according to the manufacturer's protocol. Briefly, the reaction mixture consisted of 5 pL of 4× One-step RT-ddPCR supermix for probes, 10 pL of RNA, 2 pL of reverse transcriptase, 1 pL of 300 nM DTT, 900 of nM each forward and reverse primer and 250 of nM probe. N1, N2 and RP primer/probes were synthesized from Integrated DNA Technologies (Table 5). The mixture was then used for droplet generation by adding 70 μL of Bio-Rad droplet generation oil (cat. no: 1864007). Droplets were generated with Bio-Rad QX200™ Droplet Generator. The droplets were incubated at 25° C. for 3 min, 45° C. for 60 min, 95° C. for 10 min and then cycled at 95° C. for 30 sec and at 55° C. (N1, N2,) or 60° C. (RP) for 60 sec. Amplification was performed for 45 cycles using APPLIED BIOSYSTEMS® VERITI® 96 well thermal cycler. The droplets from each sample were read on a QX200™ droplet reader machine. The data were processed using QUANTSOFT™ version 1.7.4 software.

Stability of the VLP-Based SARS-CoV-2 Positive Controls

SARS-CoV-2 positive controls (10 pg) were aliquoted to a total volume of 20 pL and stored at four different temperature (−20° C., 4° C., room temperature: 20° C.-25° C., 40° C.) for 1 h, 1 day and 1 week, respectively. RNA was extracted from VLPs and 1 pL of eluted RNA was used in RT-qPCR as mentioned previously using N1 primers/probe. All samples were assayed in triplicate on BioRad CFX96 TOUCH™ Real-Time PCR Detection System. Quantification cycle (Cq) values were tabulated by CFX™ Maestro Software.

### Example 5: Virus-Like Particles as Positive Controls for COVID-19 RT-LAMP Diagnostic Assays

Reverse transcription loop-mediated isothermal amplification (RT-LAMP) is a rapid and inexpensive isothermal alternative to the current gold standard reverse transcription quantitative polymerase chain reaction (RT-qPCR) for the detection of Severe acute respiratory syndrome coronavirus 2 (SARS-CoV-2). But unlike RT-qPCR, there are no consensus detection regions or optimal RT-LAMP methods, and most protocols do not include internal controls to ensure reliability. Naked RNAs, plasmids or even RNA from infectious COVID-19 patients have been used as external positive controls for RT-LAMP assays, but such reagents lack the stability required for full process control. To overcome the lack of proper internal and external positive controls and the instability of the detection RNA, virus-like particles (VLPs) were developed using bacteriophage Qβ and plant virus cowpea chlorotic mottle virus (CCMV) for the encapsidation of target RNA, namely a so-called SARS-CoV-2 LAMP Detection Module (SLDM). The target RNA is a truncated segment of the SARS-CoV-2 nucleocapsid (N) gene and human RNase P gene (internal control) as positive controls for RT-qPCR and RT-LAMP. Target RNAs stably encapsidated in Qβ and CCMV VLPs were previously shown to function as full process controls in RT-qPCR assays, and here it was shown that SLDMs can fulfill the same function for RT-LAMP and swab-to-test (direct RT-LAMP with heat lysis) assays. The SLDM was validated in a clinical setting, highlighting the promise of VLPs as positive controls for molecular assays.

### Results and Discussion

Design of the SARS-CoV-2 SLDM and its Encapsulation into VLPs

The SLDM was designed with N and RP detection regions consisting of primer/probe binding sites for RT-LAMP and RT-PCR. The N detection region for RT-PCR was based on CDC recommendations (N1), and the RP detection region for RT-PCR was based on the human POP7 gene encoding RNase P, which is used for RT-LAMP detection. The RT-LAMP detection regions for both genes were designed based on the Color SARS-CoV-2 LAMP Diagnostic Assay (Emergency Use Authorization (EUA) Summary for the COLOR SARS-CoV-2 LAMP Diagnostic Assay as cited here), which has been granted emergency use authorization by the FDA. This assay has a detection limit of 0.75 copies of viral RNA per microliter of primary sample and shows 100% agreement with laboratory results for 543 clinical samples; the readout measures differences in absorbance (Emergency Use Authorization (EUA) Summary for the COLOR SARS-CoV-2 LAMP Diagnostic Assay as cited here). RT-LAMP and RT-PCR should detect the same gene to allow true comparison and validation, and this is achieved in an SLDM as disclosed herein by designing both target regions with primer/probe sets for detection by RT-LAMP (assay) and RT-PCR (validation and calculation of SLDM copy number for each positive control).

The 801-nt SLDM sequence is divided into three segments (FIG. 15A): a 29-nt Qβ hairpin that links the RNA to Qβ coat proteins during encapsidation (Witherell and Uhlenbeck. Biochem. 1989, 28, 71-76), the SARS-CoV-2 N gene (accession NC_045512.2) providing the detection region for both RT-PCR (gene location 28271-28443) and RT-LAMP (gene location 29081-29320), and the RP gene (accession NM_005837.2, gene location 581-930) providing the detection region for both RT-PCR and RT-LAMP. Restriction sites Pst1 and SaI1 were placed between the sequences to facilitate subcloning and further restriction sites (Qβ 1P-C19L: Not1, Nde1; Qβ 2P-C19L: BgIII, Not1) were placed before the T7 promoter and after the T7 terminator (FIG. 15-16). There is no CDC-defined standard RT-LAMP detection region, and the design as disclosed herein allows other detection regions to be inserted in the place of those described (including the internal control detection region).

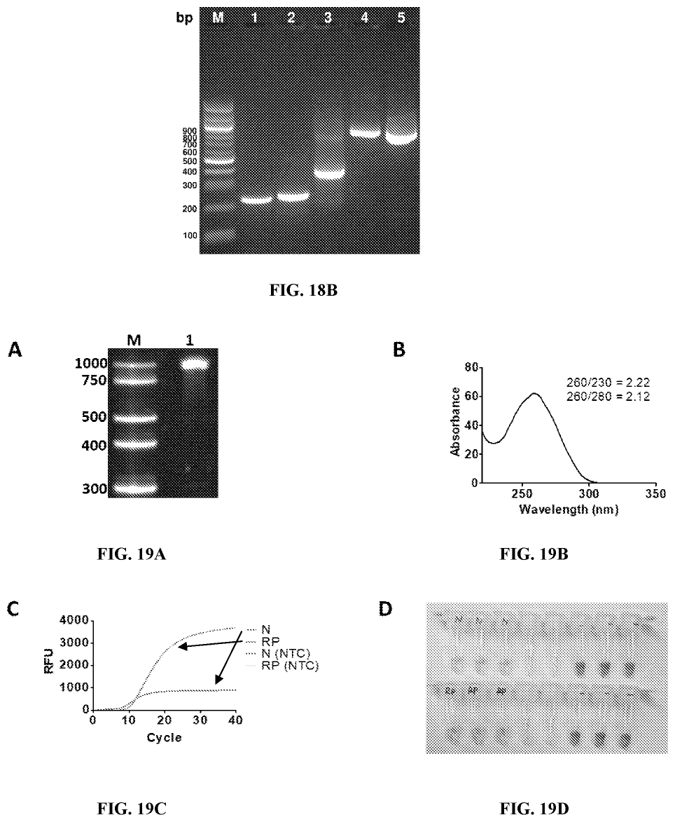

The T7 promoter and terminator drive transcription, enabling the encapsidation of SLDM RNAs into Qβ and CCMV VLPs in vivo and in vitro, respectively (FIGS. 15-16). The SLDM cassette flanked by the T7 promoter and terminator (SLDM+T7_{P/T}) was transferred to the vectors pCDFDuet-Qβ and pET-28a (+) to generate Qβ 1P-C19L and Qβ 2P-C19L, respectively (FIG. 17). The one-plasmid system (Qβ 1P-C19L) allows co-expression of the QP coat protein gene and SLDM RNA from the same vector (FIG. 18), whereas the two-plasmid system (Fang et al., 2018; and Fang et al., 2017) requires the co-transformation of bacteria with plasmids Qβ 2P-C19L and pCDFDuet-Qβ for the encapsidation of SLDM RNA in Qβ VLPs (FIG. 18). Qβ VLPs were obtained by expression in Escherichia coli (FIG. 15B), whereas CCMV-based biomimetic VLPs were obtained by the assembly of purified coat proteins (from CCMV produced in plants) with in vitro transcribed SLDM RNA (FIG. 15C).

The SLDM+T7_{P/T} cassette was intentionally misaligned with the open reading frame of the plasmid to avoid translation of the truncated SARS-CoV-2 N gene segment. The upstream ribosome binding site was likewise removed, which, not only prevents translation, but also avoids competition with the Qβ coat protein to bind the target RNA (Fang et al., 2018). The upstream ribosome binding site of the Qβ coat protein was retained for protein translation. A PstI restriction site was placed between the RT-PCR and RT-LAMP primer binding sites to further scramble the N gene sequence, as well as provide another accessible cloning site for future adaptations. The SLDM RNAs transcribed in vitro were analyzed by electrophoresis in denaturing urea polyacrylamide gels, revealing a size of ˜800 nt, as anticipated (FIG. 19A). The presence of functional binding sites in the SLDM RNA was confirmed by both RT-PCR (FIG. 19C) and RT-LAMP (FIG. 19D).

The rationale for selecting CCMV and Qβ VLPs in this study was to adopt well-established technology (rather to develop novel VLP nanotechnology) and pivot toward COVID-19 applications. Both VLPs can be produced at scale through microbial fermentation or molecular farming. Two distinct VLPs were selected: a bacteriophage and plant virus with distinct structure and RNA encapsulation mechanism. Qβ VLPs encapsidate foreign RNAs both in vivo by co-expression (Fang et al., 2017) and in vitro by assembly (Herbert, et al. Bioconjug. Chem. 2020, 31, 1529-1536); however the in vitro encapsidation method is relatively complex, requiring multiple processing steps. In contrary, in vitro encapsidation using CCMV VLPs follows a simple and well-established protocol (Comas-Garcia et al., J. Virol. 2012, 86, 12271-12282). On the downside when starting from scratch, CCMV VLP production would be delayed due to the need to establish a plant molecular farming assembly line—the first batch of CCMV could be obtained within one month starting from seed to purified and assembled CCMV VLP packaging the target RNA. In contrast, expression of recombinant Qβ VLPs is carried out overnight. Because of the differences, both systems were studied in parallel to provide comprehensive protocols and foundation to develop appropriate positive controls and RT-LAMP assays for COVID-19 diagnostics. Armored RNA technology using bacteriophage MS2 by Asuragen has demonstrated the utility of VLPs as positive controls in molecular diagnostic assays (Asuragen Develops Armored RNA Quant® SARS-CoV-2 Control as cited herein). However, Qβ VLPs outperform MS2 by having higher thermal stability (Yao et al.) and higher payload capacity (Golmohammadi et al.). Despite being well-studied, CCMV VLPs has not yet been explored as a platform technology in diagnostic assays. An advantage of the CCMV system is that in vitro encapsidation yields 100% of the target RNA being packaged; in contrast, target RNA packaged into Qβ VLPs makes only a fraction of the payload, because Qβ also packages E. coli host RNAs during assembly (Fang et al., 2017).

Validation of In Vitro Transcribed SLDM RNAs by RT-PCR and RT-LAMP

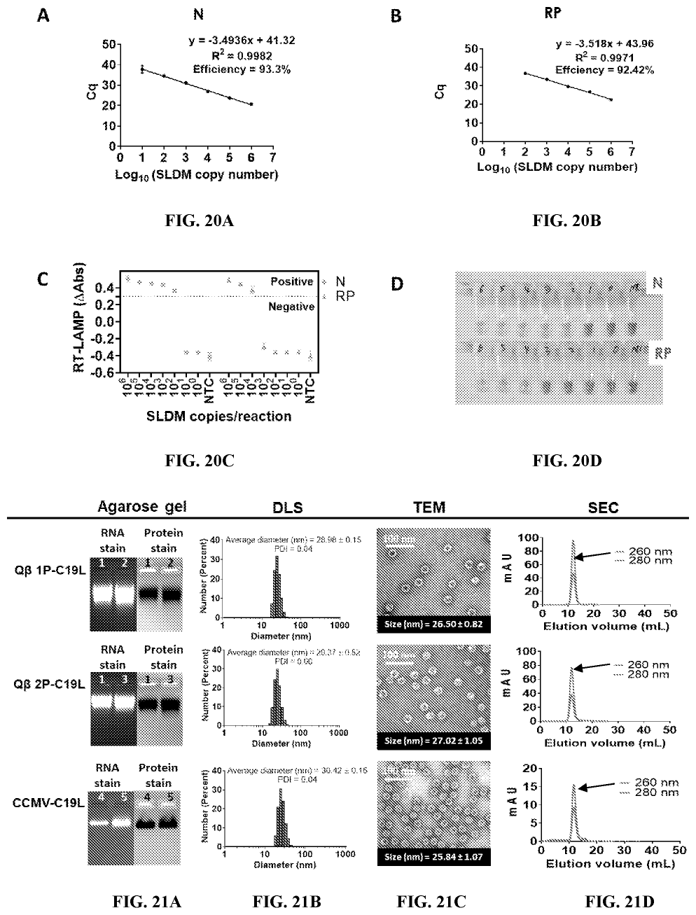

The amplification efficiency of SLDM RNA was validated by RT-qPCR using serial dilutions of the template. The amplification efficiency was calculated from the slope of standard curve using the equation: E=(10^{-1/slope}×1)×100%. See, e.g., PCR—QIAGEN available at www.qiagen.com/fi/service-and-support/learning-hub/molecular-biology-methods/per/, last accessed Nov. 10, 2020. The N and RP primer/probe sets both achieved an amplification efficiency >90% with a correlation coefficient (R²) >0.99 (FIG. 20), matching the ideal efficiency of RT-qPCR. See, e.g., Svec et al., Biomol. Detect. Quantif 2015, 3, 9-16. The primer/probe sets were also able to detect as few as 10 copies of N and 100 copies of RP with a Cq<40. The sensitivity and efficiency of the N primer/probe sets using RNA transcripts or genomic RNA extracted directly from clinical samples have been reported previously. See, e.g., Vogels et al.; and Jung et al.. The RP primer/probe set used in this study has not previously been used as a control for the detection of SARS-CoV-2 by RT-qPCR, but it was confirmed that the detection of the human RP gene in clinical samples by droplet digital PCR (ddPCR), as discussed below. Therefore, it was concluded that an SLDM RNA construct as disclosed herein was suitable as a SARS-CoV-2 positive control for RT-qPCR and for the validation of the RT-LAMP assay proposed herein.

The detection limit of the in vitro transcribed SLDM RNA was higher in the RT-LAMP assay than the RT-qPCR, a phenomenon observed in some studies (Baek et al.; and Dao et al.) but not others. See, e.g., Hu et al., mSphere 2020, 5, e00808-20; and Ling Lau et al., PeerJ 2020, 8, e9278. The difference in detection limit reflects the use of different sets of primers with different specificities for each assay. As discussed above, the RT-LAMP triggered a color change from pink to yellow in the presence of target RNAs due to the release of H⁺ ions from DNA amplification, and a positive result was defined as a difference in absorbance values at 434 and 560 nm of ≥0.3, as previously described (Dao et al.). The SLDM detection in RT-LAMP assays was 100 copies using the N primers and 10⁴ copies using the RP primers, equivalent to RT-qPCR Cq values of 35 and 30, respectively (FIGS. 20C-20D). The SLDM RNA has a much higher detection limit using primers from Color Genomics, perhaps reflecting differences in the construct as well as the RNA extraction method, final elution volume, and detection method. However, the higher detection limit can be overcome by calculating the quantity of VLPs needed to achieve an SLDM RNA load that provides a sufficient positive control.

Production and Characterization of VLP-Based SLDM Positive Controls

The VLP-based SLDM positive controls were characterized by native agarose gel electrophoresis, dynamic light scattering (DLS), transmission electron microscopy (TEM), and size exclusion chromatography (SEC), as shown in FIG. 21. All three VLP formats were intact, monodisperse particles, devoid of free nucleic acids or proteins. Accordingly, the VLPs showed matching band patterns when stained with GELRED™ (RNA stain) and Coomassie Brilliant Blue (protein stain), confirming the particle intactness. The hydrodynamic size of the Qβ 1P-C19L and Qβ 2P-C19L particles was ˜29 nm (polydispersity index=0.04-0.06) and that of the CCMV-C19L particles was ˜30 nm (polydispersity index=0.04), similar to the corresponding wild type viruses (Chan et al.). TEM images revealed particles slightly smaller than the corresponding DLS measurements due to differences in particle states (dried and stained in TEM vs. hydrated in DLS) specifically 26-27 nm for Qβ 1P-C19L and Qβ 2P-C19L, and 26 nm for CCMV-C19L, again similar to the wild type viruses (Chan et al.; and Cadena-Nava et al.). The structural integrity of all particles was confirmed by the concurrent SEC elution profiles of nucleic acid (260 nm) and protein (280 nm) from the Superose 6 column at ˜12 mL, with no additional peaks representing free RNA or protein (Doucet et al. and Cai et al.).

RNA was extracted from the VLPs using the QIAamp Viral RNA Mini Kit recommended by the CDC, which includes carrier RNA (assumed to be present at the same concentration for all extracts). The total nucleic acid concentration of the Qβ 1P-C19L and Qβ 2P-C19L extracts was higher than that of the Qβ VLPs, the difference representing the normalized copy number of SLDM RNAs (Table 8). CCMV-C19L extracts contained the lowest concentration of total nucleic acids but the highest normalized copy number of SLDM RNAs (Table 8). These results agreed with the previous findings (Chan et al.). The total nucleic acids extracted from VLPs were highly pure, with 260/230 and 260/280 ratios >2.

TABLE 8
Total nucleic acids extracted from VLP-based SARS-CoV-2 positive
controls and the proportion represented by SLDM RNA.

| | | Qβ 1P- | Qβ 2P- | CCMV- |
|---|---|---|---|---|
| | Qβ | C19L | C19L | C19L |
| Total nucleic acids (ng/μL)* | 116.03 ± 6.26 | 125.83 ± 11.20 | 124.63 ± 10.62 | 105.83 ± 19.40 |
| 260/280 | 3.09 ± 0.04 | 3.08 ± 0.02 | 3.04 ± 0.05 | 3.19 ± 0.06 |
| 260/230 | 2.62 ± 0.46 | 2.28 ± 0.27 | 2.47 ± 0.50 | 2.47 ± 0.51 |
| Normalized copy number | n/a | 9.39 ± 0.11 | 8.93 ± 0.06 | 9.74 ± 0.25 |
| (10\hat{ })** | | | | |
| Proportion of SDM RNAs in | n/a | 21.00 | 7.30 | 47.00 |
| total RNAs (%) | | | | |

*Total nucleic acids include carrier RNA.
**Normalized copy number for every 4.5 ng of total nucleic acids was tabulated to obtain quantity of SLDM RNAs as a proportion of total RNAs.

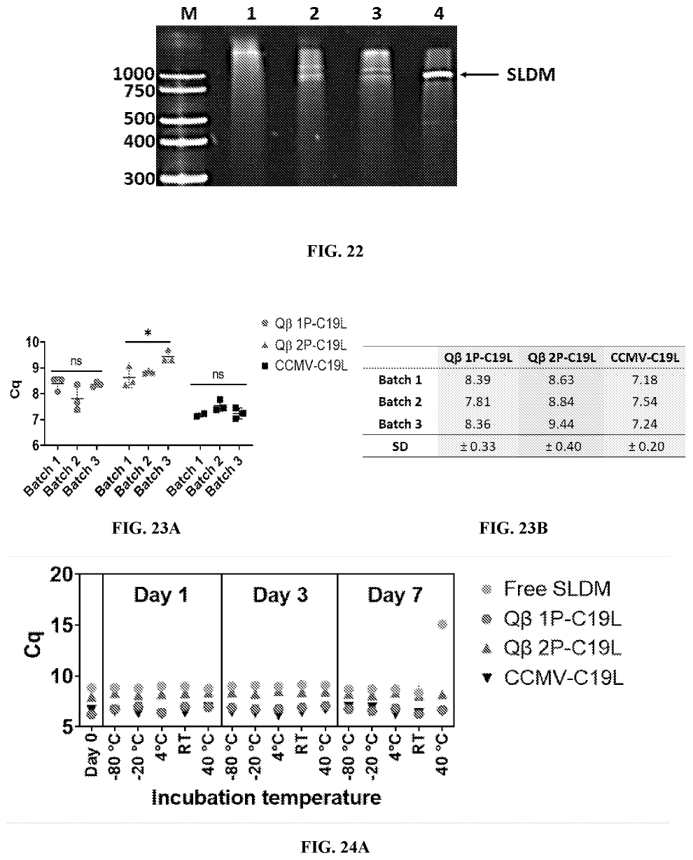

The SLDM copy number was calculated based on the standard curves (FIGS. 20A-20B). CCMV-C19L particles contained the highest SLDM copy number (10^{9.74} copies of SLDM RNA per particle, equivalent to ˜47% of total nucleic acids in the extract) followed by Qβ 1P-C19L (10^{9.39} copies, 21%) and Qβ 2P-C19L (10^{8.93} copies, 7.3%), in agreement with the earlier report (Chan et al.). CCMV-C19L was expected to encapsidate the largest quantity of SLDM RNA, because the CCMV-C19L particles were assembled with pure in vitro transcribed SLDM RNAs, which were subsequently detected in the extracts (FIG. 22). In contrast, SLDM RNAs competed with E. coli RNA to be encapsidated in the Qβ 1P-C19L and Qβ P2-C19L particles, resulting in a lower SLDM RNA copy number. The higher SLDM copy number in Qβ 1P-C19L compared to Qβ 2P-C19L particles reflected the higher transcription and/or encapsidation efficiency of the one-plasmid system (Chan et al.).

To determine the batch-to-batch consistency of the VLPs, SLDM RNAs were extracted from three separate batches and the amount of SLDM per VLP was determined by RT-qPCR. CCMV-C19L demonstrated the batch-to-batch consistency with the lowest standard deviation (±0.20) in Cq value (FIG. 23). This is expected as the amount of SLDM RNA loaded into each particle has been carefully adjusted to neutralize the basic N-terminus of CCMV coat proteins. Furthermore, only in vitro transcribed SLDM RNAs were packaged into CCMV VLPs, leading to lower batch-to-batch variability. While Qβ 1P-C19L and QP 2P-C19L also showed batch-to-batch consistency; the larger standard deviation around 0.3-0.4 in Cq values can be explained by the fact that Qβ VLPs package a mix of target and E. coli host RNAs (FIG. 23). Statistical analysis using one-way analysis of variance (ANOVA) showed that there was no significant difference comparing the batches of either CCMV-C19L and Qβ 1P-C19L. For the two-plasmid system (Qβ 2P-C19L), however, significant difference (p=0.02) between the batches were apparent. This observation is in agreement with the previous report (Chan et al.) and indicates that the two-plasmid system is less reliable. The one-plasmid system and/or CCMV VLP would be the most favorable option for scaled-up manufacture of the proposed positive controls.

Stability of VLP-Based SLDM Positive Controls

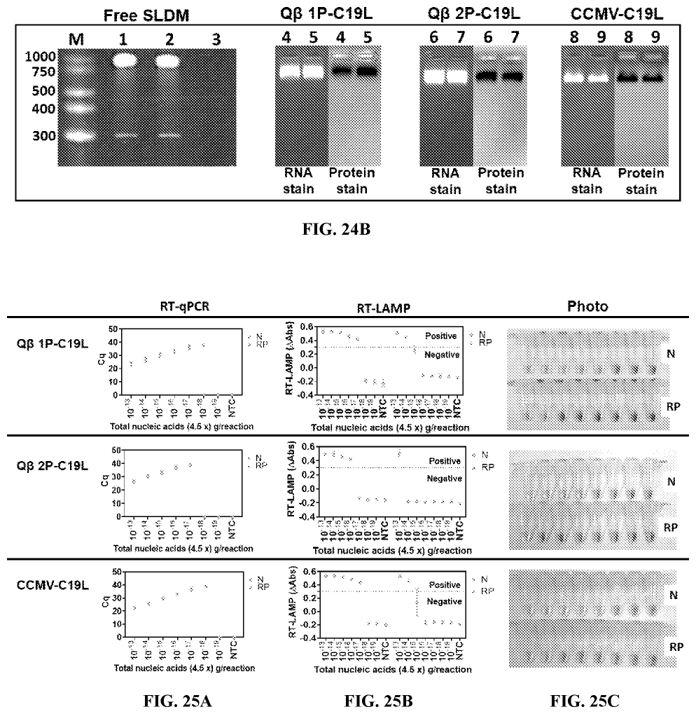

Stability of free vs. VLP packaged SLDM RNAs was assessed using two stability tests: first, free SLDM vs. VLPs (Qβ 1P-C19L, Qβ 2P-C19L, CCMV-C19L) were incubated at various temperatures (−80° C., −20° C., 4° C., room temperature: 15-20° C., 40° C.) and stability was measured over time (1 day, 3 days and 1 week); and second, free SLDM vs. VLPs (Qβ 1P-C19L, Qβ 2P-C19L, CCMV-C19L) were subjected to RNase A digestion. For longitudinal stability assays, conditions were chosen to mimic temperature ranges experienced during shipping and storage. Approximately 98% of free SLDM RNA was degraded when incubated for one week at 40° C. (FIG. 24A; significant reduction in Cq value from 8.85±0.15 at Day 0 to 15.08±0.53 at Day 7); however no significant degradation was observed for other conditions tested (with Cq values remaining consistent around 8.86±0.20). In contrast, all three positive controls (Qβ 1P-C19L, Qβ 2P-C19L, CCMV-C19L) remained stable during any condition and over the 1-week time course (with standard deviation of Cq values around +0.16 to +0.26). The small range in Cq values is most likely explained by pipetting and other handling inconsistencies (Chan et al.; and Seelenfreund et al., PLoS One 2014, 9, el 11827). From this data it is clear that the VLPs improved the thermal stability of free SLDM RNAs and this could be explained by two factors: first, the proteinaceous capsid may limit transfer of water molecules or ions that are detrimental to RNA, therefore, limiting the rate of RNA degradation inside the VLPs. In fact, it was previously shown that incubation of VLPs at ambient temperature for a month retained 70% of the RNA cargos (Chan et al.). Without the protection by the protein shell, free SLDM RNAs are vulnerable to RNA hydrolysis. Second, SLDM RNAs packaged inside the VLPs form more condensed structures and since the rate of RNA degradation is correlated to RNA structure, reduced RNA degradation is explained by increased RNA compactness inside the VLPs. See, e.g., Fang et al., 2018; and Gopal et al., PLoS One 2014, 9, e105875.

Next, the stability of free SLDM RNAs and VLP encapsidated SLDM RNAs was assayed by challenging the samples to RNase A digests at 37° C. for 30 min. As expected, free SLDM RNAs were susceptible to RNase degradation and the RNA was lost after RNase A treatment (FIG. 24B, Lane 3). When incubated under the same conditions but without the addition of RNase A, the SLDM RNAs remained intact as shown by the RNA band at ˜800 nt (FIG. 24B, Lane 1 fresh SLDM RNAs vs. Lane 2 SLDM RNAs incubated at 37° C. for 30 mins). In stark contrast, packaging the SLDM RNA into the VLPs protected the nucleic acid cargo from enzymatic degradation (FIG. 24B, Lane 5, 7, 9). This is consistent with previous reports highlighting that VLPs protect their RNA cargos from nuclease degradation. See, e.g., Fang et al., 2018; Biddlecome et al.; Comas-Garcia et al.; and Zhang et al.). Together this data highlights the benefits of encapsidating naked/free RNA into VLPs. Packing of the SLDM RNAs into VLPs conferred higher thermal stability and rendered the cargo resistant to nuclease attack.

Validation of the VLP-Based SLDM Positive Controls

Serial dilutions of total nucleic acids extracted from Qβ 1P-C19L and CCMV-C19L VLPs were validated by using RT-qPCR and RT-LAMP to detect SLDM RNA (FIG. 25). Given the presence of carrier RNA in the extracts, the detection of the N and RP regions by RT-LAMP required the presence of at least 4.5×10⁻¹⁴ g of total nucleic acids. Validation of the Qβ 2P-C19L VLPs required a 10-fold higher quantity of total nucleic acids (˜4.5×10⁻¹³ g) due to the lower quantity of encapsidated SLDM per particle. The RT-LAMP detection limit for the N and RP regions corresponded to RT-qPCR Cq values of ˜36 and ˜27, respectively. This was similar to the results obtained from the in vitro transcribed SLDM RNAs (see FIG. 20).

The Swab-to-Test RT-LAMP Assay

The swab-to-test assay allows the direct testing of samples without RNA extraction, thus reducing the need for laboratory reagents and consumables. Heating clinical samples to 95° C. for 5 min has been shown to release sufficient RNA for analysis. See, e.g., Dao et al.; Smyrlaki et al., Nat. Commun. 2020, 11, 1-12; and Rabe and Cepko. Proc. Natl. Acad. Sci. U.S.A. 2020, 117, 24450-24458. Therefore a similar strategy was adopted to test VLP-based SLDM positive controls for RNA isolation. Indeed, RNA is released more efficiently from VLPs by thermal lysis than treatment with a commercial extraction kit. See, e.g., Yu et al., J. Clin. Microbiol. 2008, 46, 837-841; Crone et al. Nat. Commun. 2020, 11, 1-11; and Hasan et al. PLoS One 2020, 15, e0236564.

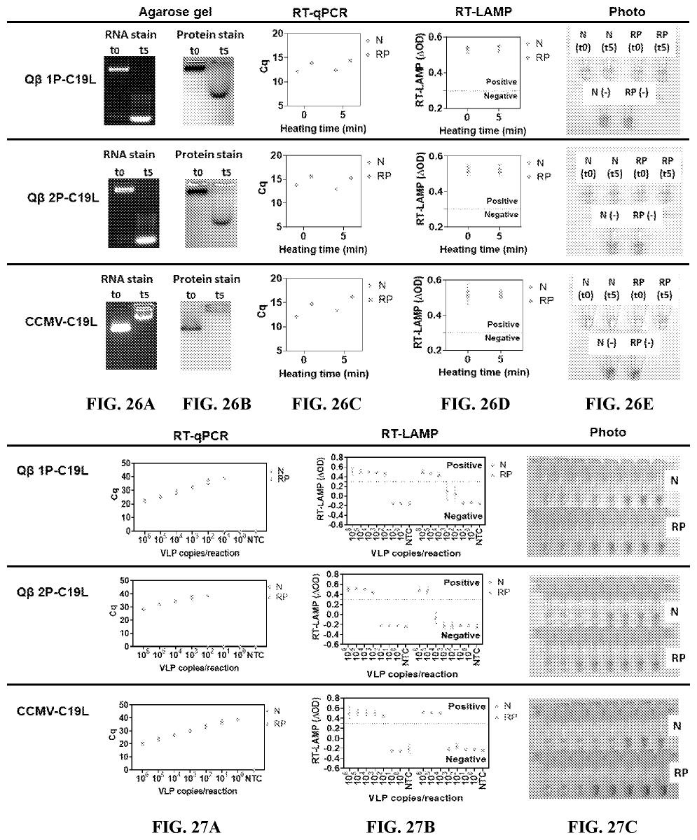

The VLPs were analyzed by native agarose gel electrophoresis before (t=0 min) and after (t=5 min) heating the particles to 95° C. The RNA and protein bands were coincident at t=0 but separate RNA and protein bands were observed at t=5, confirming the release of RNA from the particles (FIG. 26). The RNA bands at both time points were excised from the gels for analysis by RT-qPCR and RT-LAMP, revealing the presence of SLDM RNAs at both time points for all three VLP formats. The quantity of RNA released from the VLPs was similar to the quantity present in the intact particles, suggesting that heating to 95° C. releases RNA efficiently and completely.

Next, the minimum VLP copy number required was determined for the swab-to-test assay combined with RT-qPCR and RT-LAMP as detection methods (FIG. 27). A minimum of 10 copies of CCMV-C19L was required to detect the N and RP regions by RT-qPCR, whereas 100 copies of Qβ 1P-C19L and 1000 copies of Qβ 2P-C19L were required due to the lower number of SLDM RNAs encapsidated in these particles. At least 10⁴ CCMV-C19L and Qβ 1P-C19L VLPs were required to release sufficient SLDM RNA for the detection of both the N and RP regions by RT-LAMP due to the lower sensitivity of this method compared to RT-qPCR. Qβ 2P-C19L particles contained the lowest number of SLDM RNA molecules, and 10⁵ VLPs were therefore required to detect both the N and RP regions. A minimum of 10⁵ VLPs is therefore required for the successful detection of both the N and RP regions in a swab-to-test assay with the proposed primer sets and SLDM RNA, equating to 0.45 pg of QP 1P-C19L or Qβ 2P-C19L and 0.65 pg of CCMV-C19 for each assay. The RT-LAMP detection limit for the N and RP regions corresponded to RT-qPCR Cq values of ˜35 and ˜29, respectively. These observations are congruent with the findings reported above (FIGS. 20 and 25).

Validation of the VLP-Based SLDM Positive Controls in a Clinical Setting

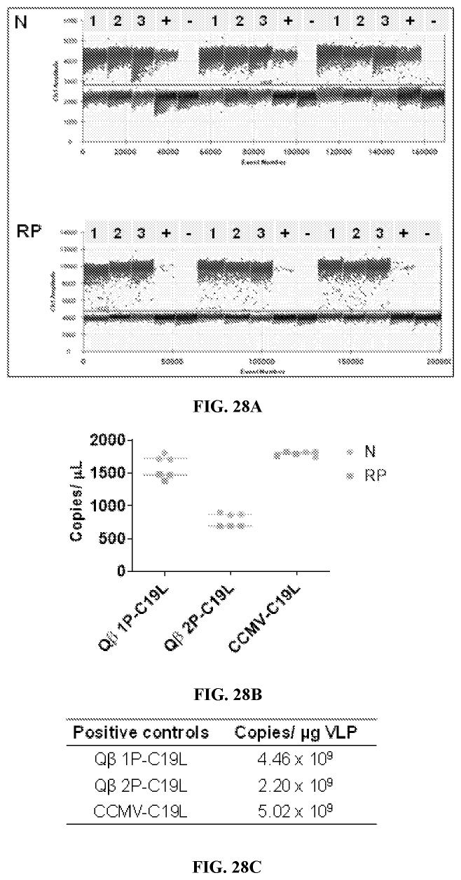

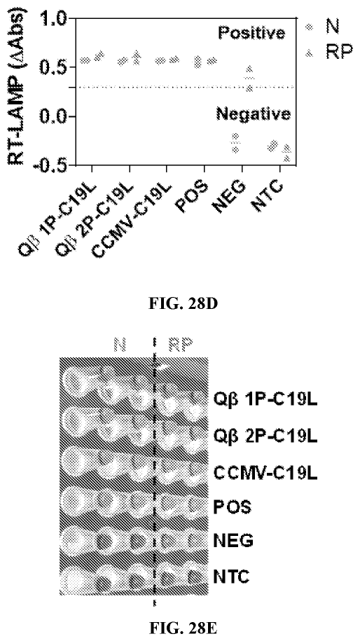

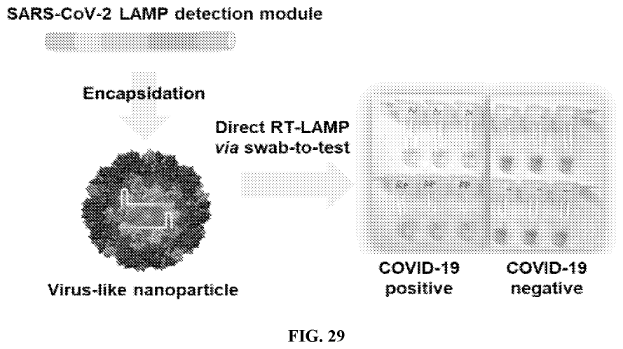

Finally, ddPCR was used to tabulate the absolute SLDM copy number extracted from positive controls alongside clinical samples from a COVID-19 patient and a healthy patient. All SLDM positive controls yielded amplitude signals of 3000-5000 for the N region and 5000-10,000 for the RP region (FIG. 28A). The N and RP regions were detected in the COVID-19 sample, confirming their suitability as RT-qPCR targets and for the validation of the RT-LAMP assay.

CCMV-C19L contained the highest SLDM copy number (1700/μL), followed by QP 1P-C19L (1500-1700/μL) and Qβ 2P-C19L (<1000/μL) based on the average copy number of both N and RP (FIG. 28B). Each microgram of CCMV-C19L particles therefore yielded ˜5.02×10⁹ SLDM molecules compared to 4.46×10⁹ for Qβ 1P-C19L and 2.20×10⁹ for QP 2P-C19L (FIG. 28C). These results are congruent with the RT-qPCR data showing that CCMV-C19L encapsidated the most SLDM RNAs and that the Qβ one-plasmid system encapsidated ˜50% more SLDM RNAs than the two-plasmid system (Table 8). Similar results were obtained for the encapsidation of SARS-CoV-2 Detection Module RNAs (Chan et al.).

SLDM positive controls were also validated for the swab-to-test assay in a clinical setting. All positive controls were readily detected (above the detection limit of 0.3) together with the sample from the COVID-19 patient, with an apparent viral load of ˜10⁵ copies/μL (FIG. 27D). This suggests the positive controls are reliable for direct RT-LAMP assays in which RNA extraction is rendered unnecessary by heating the samples to 95° C. The color change in the RT-LAMP assay was clearly detected by the naked eye (FIG. 28E).

### Conclusion

It was shown that Qβ and CCMV VLPs encapsidating designer RNAs can be used as full process positive controls in molecular assays for the detection of SARS-CoV-2. The RNAs can be extracted from the VLPs alongside clinical samples using routine methods. Three different VLP formats were tested and it was found that CCMV-C19L particles accommodated the largest quantity of SLDM RNAs (5.02×10⁹ copies) followed by Qβ 1P-C19L (4.46×10⁹) and Qβ 2P-C19L (2.20×10⁹). Due to the presence of carrier RNA in the recommended extraction kit, a minimum of 10⁻¹³ g total nucleic acids was required to detect both the N and RP regions in RT-LAMP assays. It was also showed that the RP detection region could function as both an external and an internal positive control, a standard that is absent in many publications, thus far. The proposed RP binding region (human POP7 gene) is an ideal target for both RT-LAMP and RT-PCR assays. VLP-based SLDM positive controls were not only suitable as positive controls in standard RT-LAMP assays, but also in swab-to-test assays in which samples are heated to circumvent the RNA extraction step. Heat resulted in the denaturation and disassembly of the VLPs, releasing the cargo RNAs into solution. A minimum of 10⁵ VLPs was required for optimal performance as a positive control in the swab-to-test assay. VLP-based controls as disclosed herein increase thermal stability and protect the RNA cargoes from enzymatic degradation. The VLPs are therefore suitable as positive controls for RT-LAMP point-of-care diagnostic systems as well as at-home test kits to achieve reliable and scalable mass testing.

### Materials and Methods

Assembly of the SLDM by PCR

The SLDM was constructed by assembly PCR, which was used to join fragments 1, 2 and 3, as shown in FIG. 18. All primers and gene fragments were synthesized by Eurofins Genomics (Table 9). Fragment 1 was amplified from previous SARS-CoV-2 Detection Module (Chan et al.) using primers Frag 1 Fw and Frag 1 Rv. Both strands of fragment 2 (N1 and N2) were synthesized de novo. Fragment 3 was synthesized in three parts (RP 1, RP 2 and RP 3) with overlapping regions of 12-19 bp for assembly PCR using primers Frag 3 Fw and Frag 3 Rv. The final SLDM construct was prepared by mixing 1 pL (10 ng) of each fragment followed by amplification using primers Frag 1 Fw and Frag 3 Rv. Assembly PCR was carried out using Q5 High-Fidelity 2× Master Mix (New England Biolabs) in a 25 pL reaction. The reaction was heated to 98° C. for 30 s, followed by 30 cycles of 98° C. for 10 s, 60° C. for 30 s, and 72° C. for 30 s, and a final extension step at 72° C. for 2 min.

Primer Design for RT-LAMP Assays

The RT-LAMP primers were designed according to the FDA-approved Color SARS-CoV-2 LAMP Diagnostic Assay (Emergency Use Authorization (EUA) Summary for the COLOR SARS-CoV-2 LAMP Diagnostic Assay and Abbott RealTime SARS-CoV-2 as cited herein). All primers were synthesized by Eurofins Genomics (Table 9).

Construction of Plasmids Qβ 1P-C19L and Qβ 2P-C19L

For the one-plasmid system, the SLDM was cloned into vector pCDFDuet-Qβ between restriction sites NotI and NdeI to generate Qβ 1P-C19L (FIG. 17). The insert was placed downstream of the Qβ coat protein gene and was deliberately placed out-of-frame to avoid translation of the N region. For the two-plasmid system, the SLDM was transferred to plasmid pET-28a(+) between restriction sites BgIII and NotI to generate Qβ 2P-C19L (FIG. 17). The upstream ribosome binding site was removed to avoid translation of the N region. All clones were verified by sanger sequencing (Eurofins Genomics).

Production of Qβ1P-C19L and Qβ2P-C19L VLPs

The Qβ 1P-C19L and Qβ 2P-C19L plasmids were introduced into E. coli BL21 (DE3) competent cells (New England Biolabs) and plated on LB medium containing 100 μg/mL streptomycin (Qβ 1P-C19L) or 100 μg/mL streptomycin plus 50 μg/mL kanamycin (Qβ 2P-C19L). VLPs were expressed and recovered from the cells, as previously described (Chan et al.).

Expression of SLDM by In Vitro Transcription

The SLDM was amplified using primers Frag 1 Fw and Frag 3 Rv, and the linearized construct was transcribed in vitro using the MEGASCRIPT™ T7 Transcription Kit (Thermo Fisher Scientific). The SLDM RNA was purified using the MEGAclear Transcription Clean-Up Kit (Thermo Fisher Scientific). The concentration and purity of the SLDM RNA were determined by measuring the absorbance ratio at 260/280 and 260/230 nm using a Nanodrop 2000/2000c spectrophotometer (Thermo Fisher Scientific). The integrity of the RNA was confirmed by visualization on a Novex 6% urea TBE gel (Thermo Fisher Scientific).

Production of CCMV-C19L VLPs

CCMV-infected Vigna unguiculate leaves were harvested 12 days post-infection (protocols carried out under USDA-approved P526 permits) followed by CCMV purification, as previously described. See, e.g., Bancroft. The Self-Assembly of Spherical Plant Viruses. In Advances in Virus Research; M. S., K., Max, A. L., Frederik, B. B., Eds.; Academic Press: New York, 1970; Vol. 16, pp 99-134. CCMV coat proteins were obtained by disassembly (Cadena-Nava et al.; and Annamalai and Rao) and were reassembled with in vitro transcribed SLDM RNA with 6:1 mass ratio (Cadena-Nava et al.). The concentration of reconstituted CCMV was determined using a PIERCE™ BCA protein assay kit (Thermo Fisher Scientific).

Characterization and Validation of VLP-Based SLDM Positive Controls

The VLPs were characterized by TEM, DLS, agarose gel electrophoresis, and SEC, as previously reported (Chan et al.). For validation as positive controls, SLDM RNA was extracted using the QIAamp Viral RNA Mini Kit (Qiagen) and amplified by RT-qPCR using the SuperScript III Platinum One-step RT-qPCR kit (Thermo Fisher Scientific) on a CFX96 Touch Real-Time PCR Detection System (Bio-Rad). Amplification efficiency in the N region was determined using the N1 primer/probe set 2019-nCoV CDC qPCR Probe Assay (Integrated DNA Technologies, cat. no. 10006713). Amplification efficiency in the RP region was determined using the TaqMan Gene Expression Assay with FAM probes (Applied Biosystems, assay ID Hs01921656_si). In each case, serially diluted samples contained 10⁶-10⁰ copies of SLDM RNA. All samples were tested in triplicate. Quantification cycle (Cq) values were tabulated using CFX Maestro Software.

Stability of VLP-Based SLDM Positive Controls

Incubation at various temperatures over time: Free SLDM RNAs (5 ng) and VLP-based positive controls (10 μg) were aliquoted to a total volume of 20 μL and stored at five different temperature (−80° C., −20° C., 4° C., room temperature: 15-20° C., 40° C.) for 1 day, 3 days and 1 week, respectively. RNA was extracted from VLPs as mentioned above and 1 μL of eluted RNA/free SLDM RNA was used in RT-qPCR as mentioned previously using RP primers/probe. All samples were assayed in triplicates on a BioRad CFX96 Touch real-time PCR detection system. Quantification cycle (Cq) values were tabulated by CFX Maestro software.

RNase A digestion: Free SLDM RNAs (200 ng) and VLP-based positive controls (10 μg) were incubated with 25 μg of RNase A (THERMO SCIENTIFIC™) at 37° C. for 30 mi. VLP-based positive controls were purified with AMICON© Ultra 0.5 mL centrifugal filter (100 kDa) to remove the RNase A. Purified VLP-based positive controls, with or without (control) RNase digestion (samples not subjected to RNase digestions were also incubated at 37° C. for 30 min to factor in the possibility of RNA degradation over time), were analyzed by agarose gel electrophoresis. Free SLDM RNAs, with or without RNase treatment, were analyzed by Novex TBE-Urea gels (6%; Thermo Fisher Scientific).

RNA Release Kinetics

15 pg of VLPs were incubated in a total volume of 20 pL for 5 min at 95° C. The acidic CCMV storage buffer (0.1 M sodium acetate, 1 mM EDTA, pH 4.8) was first replaced with 1×PBS buffer using Amicon Ultra-0.5 mL 100 kDa centrifugal filters to avoid the acidic buffer from triggering false positive results in the RT-LAMP assay. The concentration of CCMV-C19L in PBS was determined using the PIERCE™ BCA protein assay kit. VLP samples before and after heating were fractionated by agarose gel electrophoresis and the RNA bands excised for further analysis (Chan et al.). 1 μL of the eluted RNA was used for RT-qPCR and RT-LAMP assays using the N and RP primer/probe sets.

RT-LAMP Assays

RT-LAMP assays were carried out using WarmStart Colorimetric RT-LAMP 2× Master Mix (New England Biolabs). All primers were mixed to make a 10× stock, and the reaction was prepared with 12.5 μL WarmStart Colorimetric RT-LAMP 2× Master Mix, 2.5 μL 10× primer stock, 1 μL sample, and topped up to 25 μL with nuclease-free water (Zymo Research). All samples were tested in duplicate. The reaction mix was incubated at 65° C. for 55 min with the heated lid at 105° C. in a PTC-200 Thermal Cycler Dual 48 (MJ Research). The heated reaction mix was cooled to room temperature for 5 min, the color change was photographed, and the absorbance was measured at 434 nm and 560 nm using an Infinite 200 Rx plate reader (Tecan Life Sciences) with 25 flashes in 96-well V-bottom plate mode. The absorbance color change (ΔAbs₄₃₄₋₅₆₀) was calculated by subtracting the absorbance at 560 nm from the absorbance 434 nm.

Swab-to-Test Assay

As above, CCMV-C19L was first exchanged into PBS to avoid false positives. 10 μL of the patient samples or positive controls were heated to 95° C. for 5 min in a PCR tube and then transferred the tube immediate to a cooling block at 4° C. 1 μL of sample was added to 19 μL of RT-LAMP reaction mix (see above) in a hard-shell 96-well PCR plate (Bio-Rad), sealed the plate with a transparent heat seal (Bio-Rad) and incubated at 65° C. for 55 min with the heating lid at 105° C. The color change was recorded as above. A range of SLDM positive controls (10⁰, 10¹, 10², 10³, 10⁴, 10⁵ and 10⁶ copies) was used to investigate the detection limit of the assay and a no template control (NTC) was also included. RNA released from VLPs was also analyzed by RT-qPCR using primer/probe sets for N and RP to determine the cutoff Cq value.

Clinical Validation

For the ddPCR test, 10 μg VLP samples were extracted using the QIAamp Viral RNA mini kit and eluted in 140 μL AVE buffer. 10 μL of the eluted RNA was diluted 10⁶-fold in RNase-free water. A clinical sample from a COVID-19 patient was used as a positive control, and a sample from a healthy patient was used as a negative control. The samples were diluted 100-fold in RNase-free water. A no template control (NTC) was included to rule out contamination. Each reaction was carried out triplicate using the N and RP primer/probe sets, as previously reported (Chan et al.). For the swab-to-test assay, 10 μL of patient samples or positive controls (10⁶ copies or a minimum of 10⁵) were heated at 95° C. for 5 min, and 1 μL of the heated solution was mixed with 19 μL of RT-LAMP reaction mix as described above.

TABLE 9
Primers and oligonucleotides used to construct the SARS-COV-2 loop-
mediated isothermal amplification detection module (SLDM).

| Primer/ | SEQ | |
|---|---|---|
| oligo | ID | |
| name | NO | Sequence (5′→3′) |
| Frag 1 Fw | 15 | AAG GAA AAA AGC GGC CGC TAA TAC GAC TCA CTA TAG |
| | | GG |
| Frag 1 Rv | 16 | CTT GTG TTA CCT GCA GGT GAG AGC GGT GAA CCA AGA |
| | | CGC AGT ATT ATT G |
| Frag 2 Fw | 17 | CTC TCA CCT GCA GGT AAC ACA AGC TTT CGG CAG ACG |
| | | TGG |
| Frag 2 Rv | 18 | GCT CAT CAA CAA GGT CGA CTT GAT CTT TGA AAT TTG |
| | | GAT CTT TGT CAT C |
| Frag 3 Fw | 19 | GAT CAA GTC GAC CTT GTT GAT GAG CTG GAG CCA GAG |
| | | ACC GAC |
| Frag 3 Rv | 20 | GGA ATT CCA TAT GCA AAA AAC CCC TCA AGA CCC G |
| N 1 | 21 | CGT TCT TCG GAA TGT CGC GCA TTG GCA TGG AAG TCA |
| | | CAC CTT CGG GAA CGT GGT TGA CCT ACA CAG GTG CCA |
| | | TCA AAT TGG ATG ACA AAG ATC CAA ATT TCA AAG ATC |
| | | AA |
| N 2 | 22 | TTG ATC TTT GAA ATT TGG ATC TTT GTC ATC CAA TTT |
| | | GAT GGC ACC TGT GTA GGT CAA CCA CGT TCC CGA AGG |
| | | TGT GAC TTC CAT GCC AAT GCG CGA CAT TCC GAA GAA |
| | | CG |
| RP 1 | 23 | CTT GTT GAT GAG CTG GAG CCA GAG ACC GAC ACA CGG |
| | | GAG CCA CTG ACT CGG ATC CGC AAC AAC TCA GCC ATC |
| | | CAC ATC CGA GTC TTC AGG GTC ACA CCC AAG TAA TTG |
| | | AAA AGA CAC TCC TCC ACT TAT CCC CTC CGT GAT ATG |
| | | GCT CTT CGC ATG CTG AGT ACT GGA CCT CGG ACC AGA |
| | | GCC ATG TAA GAA AAG GCC TG |
| RP 2 | 24 | TCG GAC CAG AGC CAT GTA AGA AAA GGC CTG TTC CCT |
| | | GGA AGC CCA AAG GAC TCT GCA TTG AGG GTG GGG GTA |
| | | ATT GTC TCT TGG TGG GCC CAG TTA GTG GGC CTT CCT |
| | | GAG TGT GTG TAT GCG GTC TGT AAC TAT TGC CAT ATA |
| | | AAT AAA AAA TCC TGT TGC ACT AGT GTC CTG CCA TCC |
| | | CTA GCA TAA CCC CTT GGG GC |
| RP 3 | 25 | CCT GCC ATC CCT AGC ATA ACC CCT TGG GGC CTC TAA |
| | | ACG GGT CTT GAG GGG TTT TTT G |
| Qβ 2P- | 26 | GAA GAT CTT AAT ACG ACT CAC TAT AGG G |
| C19 | | |
| Forward | | |
| Qβ 2P- | 27 | TTT TCC TTT TGC GGC CGC CAA AAA ACC CCT CAA GAC |
| C19 | | CCG TTT AGA G |
| Reverse | | |

*Frag = Fragment; Fw = Forward; Rv = Reverse; N = Nucleocapsid; RP= Ribonuclease P

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A virus-like particle (VLP) comprising a polynucleotide encapsidated in a coat protein (CP), wherein the encapsidated polynucleotide comprises:
(a) an optional first polynucleotide comprising a bacteriophage Qbeta (QB) hairpin loop with affinity for the QB coat protein (CP), or a polynucleotide complementary thereto, and
(b) any one or any two or all three of:
(i) a second polynucleotide encoding a SARS-CoV-2 N1 or a fragment thereof or a polynucleotide complementary thereto,
(ii) a third polynucleotide encoding a SARS-CoV-2 N2 or a fragment thereof or a polynucleotide complementary thereto, or
(iii) a fourth polynucleotide encoding a human RNase P (RP) or a fragment thereof or a polynucleotide complementary thereto.

2. The VLP of claim 1, wherein the VLP is derived from a bacteriophage QB and the CP comprises a QB coat protein.

3-5. (canceled)

6. The VLP of claim 1, wherein the first polynucleotide comprises any one of: AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1), or an equivalent thereof, or
wherein the second polynucleotide comprises
AAAATGTCTGATAATGGACCCCAAAATCAGCGAAATGCACCCCGCATTAC
GTTTGGTGGACCCTCAGATTCAACTGGCAGTAACCAGAATGGAGAACGCA
GTGGGGCGCGATCAAAACAACGTCGGCCCCAAGGTTTACCCAATAATACT
GCGTCTTGGTTCACCGCTCTCAC (nt 47 to nt 219 of SEQ ID
NO: 1),
or an equivalent thereof, or
wherein the third polynucleotide comprises any one of:
CTTTCGGCAG ACGTGGTCCA GAACAAACCC AAGGAAATTT
TGGGGACCAG GAACTAATCA GACAAGGAAC TGATTACAAA
CATTGGCCGC AAATTGCACA ATTTGCCCCC AGCGCTTCAG
CGTTCTTCGG AATGTCGCGC (nt 220 to nt 359 of
SEQ ID NO: 1),
GTAACACAAG CTTTCGGCAG ACGTGGTCCA GAACAAACCC
AAGGAAATTT TGGGGACCAG GAACTAATCA GACAAGGAAC
TGATTACAAA CATTGGCCGC AAATTGCACA ATTTGCCCCC
AGCGCTTCAG CGTTCTTCGG AATGTCGCGC ATTGGCATGG
AAGTCACACC TTCGGGAACG TGGTTGACCT ACACAGGTGC
CATCAAATTG GATGACAAAG ATCCAAATTT CAAAGATCAA
(nt 226 to nt 465 of SEQ ID NO: 2),
or an equivalent of each thereof, or
wherein the fourth polynucleotide comprises any one of:
ATGGGACTTC AGCATGGCGG TGTTTGCAGA TTTGGACCTG
CGAGCGGGTT CTGACCTGAA GGCTCTGCGC GGACTTGTGG
AGACAGCCGC TCACCTTGGC TATTCAGTTG TTGCTATCAA
TCATATCGTT GACTTTAAGG AAAAGAAACA GGAAATTGAA
AAACCAGTAG CTGTTTCTGA ACTCTTCACA ACTTTGCCAA
TTGTACAGGG AAAATCAAGA CCAATTAAAA TTTTAACTAG
ATTAACAATT ATTGTCTCGG ATCCATCTCA CTGCAATGTT
(nt 360 to nt 639 of SEQ ID NO: 1)
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTGGGGG
TAATTGTCTC TTGGTGGGCC CAGTTAGTGG GCCTTCCTGA
GTGTGTGTAT GCGGTCTGTA ACTATTGCCA TATAAATAAA
AAATCCTGTT GCACTAGTGT CCTGCCATCC (nt 472 to
nt 821 of SEQ ID NO: 2),
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTG (nt
472 to nt 707 of SEQ ID NO: 2),
or an equivalent of each thereof.

7.-9. (canceled)

10. The VLP of claim 1, wherein the encapsidated polynucleotide comprises any one of SEQ ID NO: 1, SEQ ID NO: 2, a complementary sequence of each thereof, or an equivalent of each thereof.

11. The VLP of claim 1, wherein the encapsidated polynucleotide is an RNA polynucleotide.

12. (canceled)

13. A system comprising:
(1) a first vector comprising:
(a) an optional first polynucleotide comprising a bacteriophage Qbeta (QB) hairpin loop with affinity for the QB coat protein (CP), or a polynucleotide complementary thereto, and
(b) any one or any two or all three of:
(i) a second polynucleotide encoding a SARS-CoV-2 N1 or a fragment thereof or a polynucleotide complementary thereto,
(ii) a third polynucleotide encoding a SARS-CoV-2 N2 or a fragment thereof or a polynucleotide complementary thereto, or
(iii) a fourth polynucleotide encoding a human RNase P (RP) or a fragment thereof or a polynucleotide complementary thereto; and
(2) a second vector comprising:
(c) a fifth polynucleotide encoding a coat protein or a polynucleotide complementary thereto.

14. The system of claim 13, wherein the first polynucleotide comprises any one of: AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1), or an equivalent thereof, or
wherein the second polynucleotide comprises
AAAATGTCTGATAATGGACCCCAAAATCAGCGAAATGCACCCCGCATTAC
GTTTGGTGGACCCTCAGATTCAACTGGCAGTAACCAGAATGGAGAACGCA
GTGGGGCGCGATCAAAACAACGTCGGCCCCAAGGTTTACCCAATAATACT
GCGTCTTGGTTCACCGCTCTCAC (nt 47 to nt 219 of SEQ ID
NO: 1),
or an equivalent thereof, or
wherein the third polynucleotide comprises any one of:
CTTTCGGCAG ACGTGGTCCA GAACAAACCC AAGGAAATTT
TGGGGACCAG GAACTAATCA GACAAGGAAC TGATTACAAA
CATTGGCCGC AAATTGCACA ATTTGCCCCC AGCGCTTCAG
CGTTCTTCGG AATGTCGCGC (nt 220 to nt 359 of
SEQ ID NO: 1),
GTAACACAAG CTTTCGGCAG ACGTGGTCCA GAACAAACCC
AAGGAAATTT TGGGGACCAG GAACTAATCA GACAAGGAAC
TGATTACAAA CATTGGCCGC AAATTGCACA ATTTGCCCCC
AGCGCTTCAG CGTTCTTCGG AATGTCGCGC ATTGGCATGG
AAGTCACACC TTCGGGAACG TGGTTGACCT ACACAGGTGC
CATCAAATTG GATGACAAAG ATCCAAATTT CAAAGATCAA
(nt 226 to nt 465 of SEQ ID NO: 2),
or an equivalent of each thereof, or
wherein the fourth polynucleotide comprises any one of:
ATGGGACTTC AGCATGGCGG TGTTTGCAGA TTTGGACCTG
CGAGCGGGTT CTGACCTGAA GGCTCTGCGC GGACTTGTGG
AGACAGCCGC TCACCTTGGC TATTCAGTTG TTGCTATCAA
TCATATCGTT GACTTTAAGG AAAAGAAACA GGAAATTGAA
AAACCAGTAG CTGTTTCTGA ACTCTTCACA ACTTTGCCAA
TTGTACAGGG AAAATCAAGA CCAATTAAAA TTTTAACTAG
ATTAACAATT ATTGTCTCGG ATCCATCTCA CTGCAATGTT
(nt 360 to nt 639 of SEQ ID NO: 1)
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTGGGGG
TAATTGTCTC TTGGTGGGCC CAGTTAGTGG GCCTTCCTGA
GTGTGTGTAT GCGGTCTGTA ACTATTGCCA TATAAATAAA
AAATCCTGTT GCACTAGTGT CCTGCCATCC (nt 472 to
nt 821 of SEQ ID NO: 2),
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTG (nt
472 to nt 707 of SEQ ID NO: 2),
or an equivalent of each thereof.

15.-17. (canceled)

18. The system of claim 13, wherein the first vector further comprises a promoter, optionally a pol II promoter directing initiation of transcription catalyzed by an RNA polymerase II, further optionally a T7 promoter, or an equivalent or mutant thereof, located 5′ to the first polynucleotide if present and all of the coding sequences, or
wherein the promoter comprises a T3, SP6 or T7 promoter optionally wherein the T7 promoter comprises TAATACGACTCACTATAGGG (nt 1 to nt 20 of SEQ ID NO: 1), or an equivalent of each thereof, or
wherein the first vector further comprises a terminator, optionally directing termination of the transcription catalyzed by the RNA polymerase II, further optionally a T7 terminator or an equivalent of mutant thereof, located 3′ to the first polynucleotide if present and all of the coding sequences, or
wherein the first vector further comprises a terminator, optionally directing termination of the transcription catalyzed by the RNA polymerase II, further optionally a T7 terminator or an equivalent of mutant thereof, located 3′ to the first polynucleotide if present and all of the coding sequences, or optionally wherein the T7 terminator comprises CTA GCA TAA CCC CTT GGG GCC TCT AAA CGG GTC TTG AGG GGT TTT TTG (nt 640 to nt 687 of SEQ ID NO: 1), or an equivalent thereof, or
wherein the first vector comprises from 5′ to 3′, a promoter, (a) if present, (b) and a terminator, or
wherein the first vector comprises any one of: SEQ ID NO: 1, SEQ ID NO: 2, a complementary sequence of each thereof, or an equivalent of each thereof, or
wherein the second vector further comprises a regulatory sequence operatively linked to the polynucleotide of (c) and directing the expression of the coat protein.

19.-39. (canceled)

40. A recombinant polynucleotide comprising:
(a) a first polynucleotide comprising a bacteriophage Qbeta (QB) hairpin loop with affinity for the QB coat protein (CP);
(b) any one or any two or all three of:
(i) a second polynucleotide encoding a SARS-CoV-2 N1 or a fragment thereof;
(ii) a third polynucleotide encoding a SARS-CoV-2 N2 or a fragment thereof, or
(iii) a fourth polynucleotide encoding a human RNase P (RP) or a fragment thereof; and
(c) an optional fifth polynucleotide encoding the QB CP.

41. A recombinant polynucleotide comprising:
(a) an optional first polynucleotide comprising a bacteriophage Qbeta (QB) hairpin loop with affinity for the QB coat protein (CP);
(b) any one or any two or all three of:
(i) a second polynucleotide encoding a SARS-CoV-2 N1 or a fragment thereof;
(ii) a third polynucleotide encoding a SARS-CoV-2 N2 or a fragment thereof;
(iii) a fourth polynucleotide encoding a human RNase P (RP) or a fragment thereof; and
(c) a fifth polynucleotide encoding a coat protein of a cowpea chlorotic mottle virus (CCMV).

42. The recombinant polynucleotide of claim 40, wherein the first polynucleotide comprises any one of: AATTGCATGTCTAACAGACGATCTTC (SEQ ID NO: 29), or AAATGC A TGTC TAA GAC AGC AT CTTC (nucleotide (nt) 21 to nt 46 SEQ ID NO: 1), or an equivalent thereof, or
wherein the second polynucleotide comprises
AAAATGTCTGATAATGGACCCCAAAATCAGCGAAATGCACCCCGCATTAC
GTTTGGTGGACCCTCAGATTCAACTGGCAGTAACCAGAATGGAGAACGCA
GTGGGGCGCGATCAAAACAACGTCGGCCCCAAGGTTTACCCAATAATACT
GCGTCTTGGTTCACCGCTCTCAC (nt 47 to nt 219 of SEQ ID
NO: 1),
or an equivalent thereof, or
wherein the third polynucleotide comprises any one of:
CTTTCGGCAG ACGTGGTCCA GAACAAACCC AAGGAAATTT
TGGGGACCAG GAACTAATCA GACAAGGAAC TGATTACAAA
CATTGGCCGC AAATTGCACA ATTTGCCCCC AGCGCTTCAG
CGTTCTTCGG AATGTCGCGC (nt 220 to nt 359 of
SEQ ID NO: 1),
GTAACACAAG CTTTCGGCAG ACGTGGTCCA GAACAAACCC
AAGGAAATTT TGGGGACCAG GAACTAATCA GACAAGGAAC
TGATTACAAA CATTGGCCGC AAATTGCACA ATTTGCCCCC
AGCGCTTCAG CGTTCTTCGG AATGTCGCGC ATTGGCATGG
AAGTCACACC TTCGGGAACG TGGTTGACCT ACACAGGTGC
CATCAAATTG GATGACAAAG ATCCAAATTT CAAAGATCAA
(nt 226 to nt 465 of SEQ ID NO: 2),
or an equivalent of each thereof, or
wherein the fourth polynucleotide comprises any one of:
ATGGGACTTC AGCATGGCGG TGTTTGCAGA TTTGGACCTG
CGAGCGGGTT CTGACCTGAA GGCTCTGCGC GGACTTGTGG
AGACAGCCGC TCACCTTGGC TATTCAGTTG TTGCTATCAA
TCATATCGTT GACTTTAAGG AAAAGAAACA GGAAATTGAA
AAACCAGTAG CTGTTTCTGA ACTCTTCACA ACTTTGCCAA
TTGTACAGGG AAAATCAAGA CCAATTAAAA TTTTAACTAG
ATTAACAATT ATTGTCTCGG ATCCATCTCA CTGCAATGTT
(nt 360 to nt 639 of SEQ ID NO: 1),
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTGGGGG
TAATTGTCTC TTGGTGGGCC CAGTTAGTGG GCCTTCCTGA
GTGTGTGTAT GCGGTCTGTA ACTATTGCCA TATAAATAAA
AAATCCTGTT GCACTAGTGT CCTGCCATCC (nt 472 to
nt 821 of SEQ ID NO: 2)
CTTGTTGATG AGCTGGAGCC AGAGACCGAC ACACGGGAGC
CACTGACTCG GATCCGCAAC AACTCAGCCA TCCACATCCG
AGTCTTCAGG GTCACACCCA AGTAATTGAA AAGACACTCC
TCCACTTATC CCCTCCGTGA TATGGCTCTT CGCATGCTGA
GTACTGGACC TCGGACCAGA GCCATGTAAG AAAAGGCCTG
TTCCCTGGAA GCCCAAAGGA CTCTGCATTG AGGGTG (nt
472 to nt 707 of SEQ ID NO: 2),
or an equivalent of each thereof, or
optionally further comprising a promoter, optionally a pol II promoter directing initiation of transcription catalyzed by an RNA polymerase II, further optionally a T7 promoter or an equivalent or mutant thereof, located 5′ to the first polynucleotide if present and all of the coding sequences of (b) and optionally wherein the promoter comprises a T3, SP6 or T7 promoter, and optionally wherein the T7 promoter comprises TAATACGACTCACTATAGGG (nt 1 to nt 20 of SEQ ID NO: 1), or an equivalent thereof, or
optionally further comprising a terminator, optionally directing termination of the transcription catalyzed by the RNA polymerase II, further optionally a T7 terminator or an equivalent of mutant thereof, located 3′ to the first polynucleotide if present and all of the coding sequences and optionally wherein the T7 terminator comprises CTA GCA TAA CCC CTT GGG GCC TCT AAA CGG GTC TTG AGG GGT TTT TTG (nt 640 to nt 687 of SEQ ID NO: 1), or an equivalent thereof.

43.-56. (canceled)

57. A double stranded polynucleotide comprising the recombinant polynucleotide of claim 40 and the polynucleotide complementary thereto.

58.-68. (canceled)

69. An isolated host cell comprising the polynucleotide of claim 40.

70.-71. (canceled)

72. A method of preparing a recombinant polynucleotide of claim 40, comprising growing the isolated host cell of claim 69 under conditions to express the polynucleotide.

73. (canceled)

74. A method of preparing a cowpea chlorotic mottle virus (CCMV) comprising a CCMV coat protein and a polynucleotide that comprises
(a) an optional first polynucleotide comprising a bacteriophage Qbeta (QB) hairpin loop with affinity for the QB coat protein (CP), or a polynucleotide complementary thereto, and
(b) any one or any two or all three of:
(i) a second polynucleotide encoding a SARS-CoV-2 N1 or a fragment thereof or a polynucleotide complementary thereto,
(ii) a third polynucleotide encoding a SARS-CoV-2 N2 or a fragment thereof or a polynucleotide complementary thereto, or
(iii) a fourth polynucleotide encoding a human RNase P (RP) or a fragment thereof or a polynucleotide complementary thereto,
the method comprising mixing the polynucleotide with a CCMV coat protein in vitro.

75.-77. (canceled)

78. A method of preparing the VLP of claim 1, comprising culturing an isolated host cell of claim 69 under conditions suitable for producing the VLP, and isolating the VLP.

79. (canceled)

80. A method for detecting a SARS-CoV-2 infection in a subject in need thereof, comprising:
(1) contacting a sample suspected of comprising the SARS-CoV-2 and isolated from the subject with a probe that generates a detectable signal when contacting with any one or any two or all three of: (i) a second polynucleotide encoding a SARS-CoV-2 N1 or a fragment thereof, (ii) a third polynucleotide encoding a SARS-CoV-2 N2 or a fragment thereof, or (iii) a fourth polynucleotide encoding a human RNase P (RP) or a fragment thereof;
(2) separately contacting the VLP of claim 1 with the probe; and
(3) measuring the detectable signal of the contacted sample of (1) and the detectable signal of the contacted VLP of (2),
wherein the measured signal of the contacted sample of (1) at a level substantially similar or higher than the measured signal of the contacted VLP of (2) indicates the subject has a SARS-CoV-2 infection.

81.-90. (canceled)

91. A kit comprising instructions for use and the polynucleotide of claim 40.
